(19) United States
(12) Patent Application Publication
(10) Pub. No.: US 2025/0288746 A1
(43) Pub. Date: Sep. 18, 2025

(54) CARTRIDGE HOLDING UNIT

(71) Applicant: medmix Switzerland AG, Haag (CH)

(72) Inventors: Kenneth Allen FOCHT, Needham, MA (US); Peter Calvin COSTELLO, Raynham, MA (US); Joachim KEITEL, Stuttgart (DE); Herbert BECHTOLD, Denkingen (DE); Daniel P SMITH, Portmouth, RI (US)

(21) Appl. No.: 18/860,190

(22) PCT Filed: Apr. 27, 2023
(86) PCT No.: PCT/EP2023/061187

§ 371 Date: Oct. 25, 2024

Related U.S. Application Data
(63) Continuation of application No. 17/837,959, filed on Jun. 10, 2022.
Application No. 17/837,959 is a continuation of application No. 17/837,969, filed on Jun. 10, 2022.
Application No. 17/837,969 is a continuation of application No. 17/837,951, filed on Jun. 10, 2022.
Application No. 17/837,951 is a continuation of application No. 17/844,415, filed on Jun. 20, 2022.
Application No. 17/844,415 is a continuation of application No. 17/890,923, filed on Aug. 18, 2022.
(60) Provisional application No. 17/981,231, filed on Nov. 4, 2022.

(30) Foreign Application Priority Data
Apr. 27, 2022 (EP) 22170342.4
Jun. 22, 2022 (EP) 22180552.6
Jul. 5, 2022 (EP) 22183157.1
Jul. 12, 2022 (EP) 22184328.7

(51) Int. Cl. A61M 5/315; A61M 5/20; A61M 5/24; A61M 5/50

(52) U.S. Cl. CPC A61M 5/31551; A61M 5/24; A61M 5/31538; A61M 5/31541; A61M 5/31585; A61M 5/31591; A61M 5/50; A61M 2005/2073

(57) ABSTRACT

A cartridge holding unit includes a cartridge container forming an outer surface of the cartridge holding unit and having an inner space, and a cartridge holder forming a tube-shaped holding section arranged in the inner space of the cartridge container. The cartridge holder firmly holds a cartridge in parallel with the cartridge holder and the cartridge holder and the cartridge container are firmly coupled to each other in respective axial orientations.

### CROSS-REFERENCE TO RELATED APPLICATION

This application is a U.S National Stage application of International Application No. PCT/EP2023/061187, filed Apr. 27, 2023, which claims priority to European Application No. 22170342.4, filed Apr. 27, 2022, U.S. application Ser. No. 17/837,959, filed Jun. 10, 2024, U.S. application Ser. No. 17/837,969, filed Jun. 10, 2024, U.S. application Ser. No. 17/837,951, filed Jun. 10, 2022, U.S. application Ser. No. 17/844,415, filed Jun. 20, 2022, European Application No. 22180552.6, filed Jun. 22, 2022, European Application No. 22183157.1, filed Jul. 5, 2022, European Application No. 22184328.7, filed Jul. 12, 2022, U.S. application Ser. No. 17/890,923, filed Aug. 18, 2022, and U.S. application Ser. No. 17/981,231, filed Nov. 4, 2022, the contents of each of which are hereby incorporated by reference.

### BACKGROUND

### Technical Field

The disclosure relates to a cartridge holding unit for a dose delivery mechanism.

### Background Information

Conventional dose delivery mechanisms can be provided in the form of a pen and can for example be used for insulin therapy or growth hormone therapy. Known pens have a cartridge container with a section for attaching a cartridge.

### SUMMARY

It has been determined that when the cartridge is attached to the cartridge container, an axial orientation of the cartridge can vary compared to an axial orientation of the cartridge container. This usually results in the axial orientation of the cartridge not being in parallel with an axial orientation of a piston and piston rod that are used to push medicament out of the cartridge during dose delivery. However, if the axial orientation of the cartridge is not in parallel with an axial orientation of the piston rod and the piston, friction between the piston and the cartridge is higher. Therefore, a higher force is needed to perform a dose delivery.

It is an object of the present disclosure to provide a cartridge holding unit that reduces a force needed to perform a dose delivery. In particular, it is an object of the disclosure to provide a cartridge holding unit that reduces a risk of a jam between a piston rod and a cartridge during dose delivery.

The object is satisfied by a cartridge holding unit as described herein. The cartridge holding unit comprises a cartridge container forming an outer surface of the cartridge holding unit and having an inner space, and a cartridge holder forming a tube-shaped holding section arranged in the inner space of the cartridge container, wherein the cartridge holder is configured to, in particular firmly, hold a cartridge in parallel with the cartridge holder and wherein the cartridge holder and the cartridge container are, in particular firmly, coupled to each other regarding their axial orientations.

The disclosure is based on the idea that a cartridge holder is provided that has a tube-shaped holding section provided inside the cartridge container. In the assembled state, the cartridge holder holds the cartridge due to the tube-shaped holding section so that their respective axial orientations are in parallel with each other. Furthermore, the cartridge holder and the cartridge container are firmly coupled, i.e. rigidly connected, to each other so that their respective axial orientations are in parallel with each other. In other words, the cartridge holder is configured to hold the cartridge relative to the cartridge holder without relative movement possible therebetween. Furthermore, the cartridge container is coupled to the cartridge holder to hold the cartridge container relative to the cartridge holder immovable with respect to one another. Thereby it follows that the cartridge and the cartridge container are in parallel with each other so that—if the piston rod and piston are arranged in parallel with the cartridge container, which is usually the case—the cartridge is arranged in parallel with the piston rod and piston. If the cartridge is arranged in parallel with the piston rod and piston, friction between an inner circumferential surface of the cartridge and an outer circumferential surface of the piston is reduced so that the force needed to perform a dose delivery is reduced.

Embodiments of the cartridge holding unit are described in the following disclosure.

According to an embodiment, the cartridge container and the cartridge holder are formed as two separate parts. This allows manufacturing the cartridge container and the cartridge holder via injection molding which allows production of the cartridge holding unit in a cost-efficient manner. Alternatively, the cartridge container and the cartridge holder can be formed integrally with each other, e.g. by 3D printing.

According to an embodiment, the cartridge container and the cartridge holder are non-detachably coupled to each other. “Non-detachably” means that the cartridge container and the cartridge holder cannot be detached by hand by applying a force that occurs with normal use. Alternatively, the cartridge container and the cartridge holder can be detachably coupled to each other.

According to an embodiment, the cartridge container and the cartridge holder are axially fixedly connected to each other. The cartridge container and the cartridge holder can be axially fixedly connected to each other by a form-locked engagement, for example by a snap fit connection. A snap fit connection allows to assemble the cartridge container and the cartridge holder in a simple, but reliant manner. Furthermore, the snap fit connection also would allow shipping the cartridge holding unit in a disassembled state to a patient since the assembling of the cartridge unit can be done without tools by the patient.

According to an embodiment, the cartridge container and the cartridge holder are firmly coupled to each other regarding their axial orientations by a form-locked engagement, for example by a snap fit connection.

To make sure that the axial orientations of the cartridge container and the cartridge holder coincide with each other, the cartridge container and the cartridge holder can each form corresponding end faces that abut each other. The end faces can be annular end faces.

According to an embodiment, the cartridge holder forms an axial fixation means that engage with an axial fixation means of the cartridge container. For example, one of the axial fixation means, such as the fixation means of the cartridge holder, is configured as a circumferentially extending groove and the other one of the axial fixation means, such as the fixation means of the cartridge container, is configured as a circumferentially extending rib. The groove and/or the rib can extend fully circumferentially around the respective part.

According to an embodiment, the cartridge holder and the cartridge container are rotationally fixed to each other.

According to an embodiment, the cartridge holder forms rotation fixation means that engage rotation fixation means of the cartridge container. The cartridge holder and the cartridge container can be rotationally fixed to each other by a form-fitting engagement between the rotation fixation means of the cartridge holder and the rotation fixation means of the cartridge container. For example, one of the rotation fixation means, such as the rotation fixation means of the cartridge holder, can be configured as a radially protruding and axially extending rib and the other one of the rotation fixation means, such as the rotation fixation means of the cartridge container, can be configured as a corresponding radially and axially extending groove for receiving the rib.

According to an embodiment, the cartridge holder forms a connector for attaching a needle assembly like a pen needle, i.e. a needle connector, at its proximal end. The needle connecter can be an inner or outer thread. The needle connector can alternatively be part of a bayonet lock or a luer lock.

According to an embodiment, the needle connector surrounds an opening at a proximal end of the cartridge holder, wherein the opening is configured to receive a cannula of the needle assembly that is in fluid connection with an interior of the cartridge inserted into the cartridge holder.

According to an embodiment, the cartridge holder forms a slot extending in the axial direction, the slot allowing to reversibly widen the cartridge holder to axially insert a cartridge. The slot can be configured to reversibly widen the cartridge holder to axially insert a cartridge having a protrusion on its outer circumferential surface. Such a cartridge can be a dual chamber cartridge having a bypass.

According to an embodiment, the slot extends until a distal end of the cartridge holder. This allows easily widening of the distal end of the cartridge holder to axially insert a cartridge having a protrusion on its outer circumferential surface.

According to an embodiment, when the cartridge holder is firmly coupled to the cartridge container, a window formed in the cartridge holder is aligned with a window in the cartridge container so that a medicament inside an at least partly transparent cartridge arranged inside the cartridge holder can be inspected through the windows. The rotation fixation means of the cartridge container and the cartridge holder can be configured so that they hold the cartridge container and the cartridge holder in a relative rotational position so that the window in the cartridge holder is aligned with the window in the cartridge container.

According to an embodiment, the holding section of the cartridge holder defines a cylindrical receptacle. Preferably, the cartridge has a cylindrical shape so that the cartridge holder and the cartridge are in areal contact with each other along the holding section of the cartridge holder.

According to an embodiment, an inner surface of the cartridge holder generally is configured to be in areal contact with the cartridge inserted into the cartridge holder. The inner surface of the cartridge holder that is configured to be in areal contact with the cartridge inserted into the cartridge holder preferably extends around a closed circumference.

According to an embodiment, the cartridge holder forms a cut-out for receiving a protrusion of an inserted cartridge, such as a bypass of a dual chamber cartridge. The cut-out can be located circumferentially offset from the longitudinal slot of the cartridge holder, such as radially opposite of the longitudinal slot of the cartridge holder. The cut-out can be configured as a window in the tube-shaped holding section.

According to an embodiment, the tube-shaped holding section can have a constant inner diameter in a non-deformed state along the axial direction. The tube-shaped holding section can have a circular inner diameter in a non-deformed state along the axial direction. Alternatively, the tube-shaped holding section can have a non-circular inner diameter, such as an oval inner diameter, so that a friction or force fit connection can be established between a deformed holding section and a cylindrically shaped cartridge.

According to an embodiment, borders of the cut-out are configured to enclose, for example fully enclose, a protrusion on the outer surface of an inserted cartridge to prevent rotation of the cartridge around its longitudinal axis and/or axial movement of the cartridge along the longitudinal axis.

According to an embodiment, the holding section is configured to extend over at least 30%, more preferably 50%, of a longitudinal length of an inserted cartridge. This allows safe coupling of the cartridge with the cartridge holder so that they are firmly coupled to each other regarding their axial orientations.

According to an embodiment, the cartridge holder has a connection means (element) for connection to a dosing mechanism. The connection means can comprise a thread, such as an inner thread. Alternatively or additionally, the connection means can comprise snap fit connection means such as a snap element or an opening engagable with a snap element. According to an embodiment, the cartridge holder can form a connection means that comprise an inner thread and at least two openings, wherein each of the openings is engagable with a same snap element of a connection section of a dosing mechanism forming an outer thread corresponding to the inner thread.

According to an embodiment, the connection means extends into an annular space in between the cartridge holder and the cartridge container. Preferably, the connection means, such as the inner thread, extends along a majority of an inner circumferential surface of the cartridge container. This allows to form the cartridge holder unit in a compact manner.

According to an embodiment, the cartridge container forms a gripping portion on its outer surface so that the user can hold the cartridge holding unit on the outer surface of the cartridge container, e.g. to rotate the cartridge container relative to a dosing mechanism.

The disclosure is also directed at a cartridge unit comprising the cartridge holding unit according to at least one of the preceding or below mentioned embodiments and a cartridge, for example a dual chamber cartridge having a bypass, inserted into the cartridge holder.

The disclosure is also directed at a reconstitution unit comprising:
a cartridge holder unit according to at least one of the aforementioned or below mentioned embodiments, and a connection section of a dosing mechanism, such as a piston rod guide, wherein the cartridge container forms a first thread, such as the inner thread of the connection means, and the connection section of the dosing mechanism forms a corresponding second thread engageable with each other to cause an axial movement of the cartridge container relative to the connection section of the dosing mechanism by rotating the cartridge container relative to the connection section of the dosing mechanism.

According to an embodiment, the first thread is formed on an inner circumferential surface of the cartridge container and the second thread is formed on an outer circumferential surface of the connection section of the dosing mechanism. This has the advantage that the outer circumferential surface of the cartridge container can be used as a gripping surface by the user to rotate the cartridge holder unit relative to the connection section, e.g. for performing a reconstitution movement.

According to an embodiment, the reconstitution unit comprises a cartridge having a protrusion, wherein the cartridge holder forms a cut-out for receiving the protrusion of the cartridge, and wherein the protrusion form-fittingly engages the cut-out so that the cartridge is axially and/or rotationally fixed to the cartridge holder.

According to an embodiment, a space is formed between an outer circumferential surface of the cartridge holder and an inner circumferential surface of the cartridge container so that the connection section of the dosing mechanism can be arranged or moved in the space between the outer circumferential surface of the cartridge holder and the inner circumferential surface of the cartridge container upon mounting the cartridge holding unit or cartridge unit to the connection section of the dosing mechanism and/or moving the connection section of the dosing mechanism relative to the cartridge holding unit or cartridge unit during use. For example, the space between the outer circumferential surface of the cartridge holder and the inner circumferential surface of the cartridge container can be configured to be occupied by the connection section of the dosing mechanism during reconstitution.

### BRIEF DESCRIPTION OF THE DRAWINGS

These and other features, aspects and advantages are described below with reference to the drawings. Like reference characters denote corresponding features consistently throughout the drawings.

FIG. 1 shows an exploded view of an injection pen according to the disclosure.

FIG. 2A shows a perspective view of a knob cover of the injection pen of FIG. 1.

FIG. 2B shows a side view of the knob cover of FIG. 2A.

FIG. 2C shows a section view of the knob cover of FIG. 2B along the line A-A of FIG. 2B.

FIG. 3A shows a perspective view of an injection button of the injection pen of FIG. 1.

FIG. 3B shows a side view of the injection button of FIG. 3A.

FIG. 3C shows a section view of the injection button of FIG. 3B along the line A-A of FIG. 3B.

FIG. 3D shows a section view of the injection button of FIG. 3B along the line B-B of FIG. 3B.

FIG. 3E shows a section view of the injection button of FIG. 3B along the line C-C of FIG. 3B.

FIG. 4A shows a perspective view of a snap ring of the injection pen of FIG. 1.

FIG. 4B shows a top view of the snap ring of FIG. 4A.

FIG. 4C shows a side view of the snap ring of FIG. 4A.

FIG. 4D shows a bottom view of the snap ring of FIG. 4A.

FIG. 5A shows a first perspective view of a dose setting knob of the injection pen of FIG. 1.

FIG. 5B shows a second perspective view of the dose setting knob of FIG. 5A.

FIG. 5C shows a side view of the dose setting knob of FIG. 5A.

FIG. 5D shows a section view of the dose setting knob of FIG. 5A along line A-A of FIG. 5C.

FIG. 6A shows a perspective view of a snap element of the injection pen of FIG. 1.

FIG. 6B shows a side view of the snap element of FIG. 6A.

FIG. 6C shows a section view of the snap element of FIG. 6A along the line A-A of FIG. 6B.

FIG. 7A shows a perspective view of a connector of the injection pen of FIG. 1.

FIG. 7B shows a bottom view of the connector of FIG. 7A.

FIG. 7C shows a side view of the connector of FIG. 7A.

FIG. 7D shows a top view of the connector of FIG. 7A.

FIG. 8A shows a first perspective view of a dose selector of the injection pen of FIG. 1.

FIG. 8B shows a bottom view of the dose selector of FIG. 8A.

FIG. 8C shows a side view of the dose selector of FIG. 8A.

FIG. 8D shows a section view of the dose selector of FIG. 8A along the line A-A of FIG. 8C.

FIG. 8E shows a section view of the dose selector of FIG. 8A along the line B-B of FIG. 8C.

FIG. 9 shows a second perspective view of the dose selector of FIG. 8A.

FIG. 10A shows a perspective view of a knob key of the injection pen of FIG. 1.

FIG. 10B shows a side view of the knob key of FIG. 10A.

FIG. 11A shows a perspective view of a housing of the injection pen of FIG. 1.

FIG. 11B shows a side view of the housing of FIG. 11A.

FIG. 11C shows a section view of the housing of FIG. 11A along the line A-A of FIG. 11B.

FIG. 12A shows a first side view of a dose setting sleeve of the injection pen of FIG. 1.

FIG. 12B shows a second side view of the dose setting sleeve of FIG. 12A.

FIG. 12C shows a third side view of the dose setting sleeve of FIG. 12A.

FIG. 12D shows a fourth side view of the dose setting sleeve of FIG. 12A.

FIG. 12E shows a front view of the dose setting sleeve of FIG. 12A.

FIG. 12F shows a first perspective view of the dose setting sleeve of FIG. 12A.

FIG. 12G shows a second perspective view of the dose setting sleeve of FIG. 12A.

FIG. 13A shows a perspective view of a driver of the injection pen of FIG. 1.

FIG. 13B shows a first side view of the driver of FIG. 13A.

FIG. 13C shows a second side view of the driver of FIG. 13A.

FIG. 13D shows a section view of the driver of FIG. 13A along the line A-A of FIG. 13C.

FIG. 14A shows a first perspective view of a nut of the injection pen of FIG. 1.

FIG. 14B shows a second perspective view of the nut of FIG. 14A.

FIG. 14C shows a side view of the nut of FIG. 14A.

FIG. 14D shows a first section view of the nut of FIG. 14A along the line A-A of FIG. 14C.

FIG. 14E shows a second section view of the nut of FIG. 14A along the line B-B of FIG. 14C.

FIG. 15A shows a first side view of a piston rod guide of the injection pen of FIG. 1.

FIG. 15B shows a second side view of the piston rod guide of FIG. 15A.

FIG. 15C shows a section view of the piston rod guide of FIG. 15A along the line A-A of FIG. 15A.

FIG. 15D shows a perspective view of the piston rod guide of FIG. 15A.

FIG. 16A shows a first longitudinal section view of the piston rod guide of FIG. 15A.

FIG. 16B shows a second longitudinal section view of the piston rod guide of FIG. 15A.

FIG. 16C shows a perspective view of the piston rod guide of FIG. 15A.

FIG. 17A shows a side view of a piston rod of the injection pen of FIG. 1.

FIG. 17B shows a section view of the piston rod of FIG. 17A along the line A-A of FIG. 17A.

FIG. 17C shows a first perspective view of the piston rod of FIG. 17A.

FIG. 17D shows a second perspective view of the piston rod of FIG. 17A.

FIG. 18A shows a perspective view of a piston disc of the injection pen of FIG. 1.

FIG. 18B shows a top view of the piston disc of FIG. 18A.

FIG. 18C shows a section view of the piston disc of FIG. 18A along the line A-A of FIG. 18B.

FIG. 19A shows a perspective view of a dual chamber cartridge of the injection pen of FIG. 1.

FIG. 19B shows a side view of the dual chamber cartridge of FIG. 19A.

FIG. 19C shows a section view of the dual chamber cartridge of FIG. 19A along the line A-A of FIG. 19B.

FIG. 20A shows a perspective view of a cartridge container of the injection pen of FIG. 1.

FIG. 20B shows a first side view of the cartridge container of FIG. 20A.

FIG. 20C shows a second side view of the cartridge container of FIG. 20A.

FIG. 20D shows a section view of the cartridge container of FIG. 20A along the line A-A of FIG. 20C.

FIG. 21A shows a first perspective view of a cartridge key of the injection pen of FIG. 1.

FIG. 21B shows a second perspective view of the cartridge key of FIG. 21A.

FIG. 21C shows a first side view of the cartridge key of FIG. 21A.

FIG. 21D shows a second side view of the cartridge key of FIG. 21A.

FIG. 22A shows a third side view of the cartridge key of FIG. 21A.

FIG. 22B shows a section view of the cartridge key of FIG. 21A along the line A-A of FIG. 22A.

FIG. 23A shows a side view of the injection pen of FIG. 1 in an as-delivered state.

FIG. 23B shows a section view of the injection pen of FIG. 23A along the line A-A of FIG. 23A.

FIG. 24 shows a perspective view of the injection pen of FIG. 23A without the knob cover and with some parts displayed transparently.

FIG. 25A shows a second side view of the injection pen of FIG. 23A.

FIG. 25B shows a section view of the injection pen of FIG. 23A along the line A-A of FIG. 25A.

FIG. 26A shows a side view of the injection pen of FIG. 1 in a reconstitution state.

FIG. 26B shows a section view of the injection pen of FIG. 26A along the line A-A of FIG. 26A.

FIG. 27A shows a second side view of the injection pen of FIG. 26A.

FIG. 27B shows a third side view of the injection pen of FIG. 26A.

FIG. 27C shows a section view of the injection pen of FIG. 26A along the line A-A of FIG. 27B.

FIG. 28A shows a side view of the injection pen of FIG. 1 in a knob cover unfastening state.

FIG. 28B shows a section view of the injection pen of FIG. 28A along the line A-A of FIG. 28A.

FIG. 29A shows a side view of the injection pen of FIG. 1 in an end of reconstitution state.

FIG. 29B shows a section view of the injection pen of FIG. 29A along the line A-A of FIG. 29A.

FIG. 30A shows a side view of the injection pen of FIG. 1 in a set dose state.

FIG. 30B shows a section view of the injection pen of FIG. 30A along the line A-A of FIG. 30A.

FIG. 31A shows a side view of the injection pen of FIG. 1 in a start of injection state.

FIG. 31B shows a section view of the injection pen of FIG. 31A along the line A-A of FIG. 31A.

FIG. 32A shows a further side view of the injection pen of FIG. 1 in a start of injection state.

FIG. 32B shows an enlarged section view of the injection pen of FIG. 32A along the line A-A of FIG. 32A.

FIG. 33A shows a side view of the injection pen of FIG. 1 in an end of injection state.

FIG. 33B shows a section view of the injection pen of FIG. 33A along the line A-A of FIG. 33A.

FIG. 34 shows a second injection pen according to the present disclosure in a preassembled state.

FIG. 35 shows another view of the second injection pen in the preassembled state.

FIG. 36 shows an exploded view of the second injection pen.

FIG. 37 shows a longitudinal cut through the second injection pen in the preassembled state.

FIG. 38 shows a detailed view of a distal portion of the second injection pen in the preassembled state.

FIG. 39 shows a detailed view of the distal end of the second injection pen in an assembled state during dose setting.

FIG. 40 shows another view of the distal end of the second injection pen in the assembled state during dose setting.

FIG. 41 shows a detailed view of the distal end of the second injection pen in an assembled state during dose delivery.

FIG. 42 shows another view of the distal end of the second injection pen and the assembled state during dose delivery.

FIG. 43 shows a longitudinal cut through the dose setting element of the first and second injection pen.

FIG. 44 shows a perspective view of the longitudinal cut through the dose setting element.

FIG. 45 shows a perspective distal view of the dose setting element.

FIG. 46 shows a prospective proximal view of the dose setting element.

FIG. 47 shows a perspective view of a third injection pen according to the present disclosure.

FIG. 48 shows an exploded view of the third injection pen.

FIG. 49 shows a longitudinal cut through a dose delivery mechanism of the third injection pen.

FIG. 50 shows a perspective distal view of a dosing member of the third injection pen.

FIG. 51 shows a perspective longitudinal cut through the dosing member of the third injection pen.

FIG. 52 shows a perspective view of a piston rod of the third injection pen.

FIG. 53 shows a perspective distal view of an extension of the third injection pen.

FIG. 54 shows a distal view of the extension shown in FIG. 53.

FIG. 55 shows a proximal view of the extension shown in FIG. 53.

FIG. 56 shows a perspective view of a coupling element of the third injection pen.

FIG. 57 shows a perspective view of a bearing, the piston rod, the extension, the coupling element, and an adjusting element of the third injection pen

FIG. 58 shows a proximal perspective view of the adjusting element of the third injection pen.

FIG. 59 shows a side view of the adjusting element of the third injection pen.

FIG. 60 shows a radial cut through the adjusting element of the third injection along the line A-A in FIG. 59.

FIG. 61 shows a radial cut through the adjusting element of the third injection along the line B-B in FIG. 59.

FIG. 62 shows a perspective view of a coupling member of the third injection pen.

FIG. 63 shows a perspective distal view of a sleeve of the third injection pen.

FIG. 64 shows a longitudinal cut through the sleeve of the third injection pen.

FIG. 65 shows a perspective view of a housing insert of the third injection pen.

FIG. 66 shows a perspective view of a connector of the third injection pen.

FIG. 67 shows a perspective view of a longitudinal cut through the connector of the third injection pen.

FIG. 68 shows a side view of the third injection pen in an assembled state during dose setting.

FIG. 69 shows a side view of the third injection pen in a preassembled state.

FIG. 70 shows a detailed view of a longitudinal cut through the distal end of the third injection pen in an assembled state during dose setting.

FIG. 71 shows a detailed view of a further longitudinal cut through the distal end of the third injection pen in the assembled state during dose setting.

FIG. 72 shows a detailed view of a longitudinal cut through the distal end of the third injection pen in the assembled state during dose delivery.

FIG. 73 shows a detailed view of a further longitudinal cut through the distal end of the third injection pen in the assembled state during dose delivery.

FIG. 74 shows a detailed view of a longitudinal cut through the distal end of the third injection pen in the preassembled state.

FIG. 75 shows a detailed view of a further longitudinal cut through the distal end of the third injection pen in the preassembled state.

FIG. 76 shows a perspective view of a fourth injection pen according to the present disclosure.

FIG. 77 shows a side view of the fourth injection pen in an assembled state during dose setting.

FIG. 78 shows a side view of the fourth injection pen in a preassembled state with an adjusting element in a preassembled position.

FIG. 79 shows a side view of the fourth injection pen in the preassembled state with the adjusting element in an adjusting position.

FIG. 80 shows an exploded view of the fourth injection pen.

FIG. 81 shows a longitudinal cut through a dose delivery mechanism of the fourth injection pen in the assembled state during dose setting.

FIG. 82 shows a perspective view of a coupling element of the fourth injection pen.

FIG. 83 shows a radial cut through the coupling element shown in FIG. 82 along the line A-A.

FIG. 84 shows a perspective view of a piston rod of the fourth injection pen.

FIG. 85 shows a perspective proximal view of an adjusting element of the fourth injection pen.

FIG. 86 shows a side view of the adjusting element of the fourth injection pen.

FIG. 87 shows a first longitudinal cut through the adjusting element along the line A-A in FIG. 86.

FIG. 88 shows a further side view of the adjusting element of the fourth injection pen in a direction perpendicular to the direction of FIG. 86.

FIG. 89 shows a second longitudinal cut through the adjusting element along the line BB in FIG. 88.

FIG. 90 shows a perspective distal view of a sleeve of the fourth injection pen.

FIG. 91 shows a perspective distal view of an insert of the sleeve.

FIG. 92 shows a perspective proximal view of the insert of the sleeve.

FIG. 93 shows a perspective distal view of an outer part of the sleeve.

FIG. 94 shows a side view of a coupling member of the fourth injection pen.

FIG. 95 a longitudinal cut through the coupling member along the line A-A in FIG. 90.

FIG. 96 shows a radial cut through the coupling member along the line B-B in FIG. 90.

FIG. 97 shows a perspective distal view of a dosing member of the fourth injection pen.

FIG. 98 shows a perspective view of a longitudinal cut through the dosing member.

FIG. 99 shows a longitudinal cut through the distal end of the dose delivery mechanism of the fourth injection pen in the assembled state during dose setting.

FIG. 100 shows a further longitudinal cut through the distal end of the dose delivery mechanism of the fourth injection pen in the assembled state during dose setting.

FIG. 101 shows a longitudinal cut through the distal end of the dose delivery mechanism of the fourth injection pen in the assembled state during dose delivery.

FIG. 102 shows a further longitudinal cut through the distal end of the dose delivery mechanism of the fourth injection pen in the assembled state during dose delivery.

FIG. 103 shows a longitudinal cut through a distal end of a dose delivery mechanism of the fourth injection pen in a preassembled state with the adjusting element in an preassembled position.

FIG. 104 shows a further longitudinal cut through the distal end of the dose delivery mechanism of the fourth injection pen in the preassembled state with the adjusting element in the preassembled position.

FIG. 105 shows a longitudinal cut through the distal end of the dose delivery mechanism of the fourth injection pen in the preassembled state with the adjusting element in an adjusting position.

FIG. 106 shows a further longitudinal cut through the distal end of the dose delivery mechanism of the fourth injection pen in the preassembled state with the adjusting element in the adjusting position.

### DETAILED DESCRIPTION

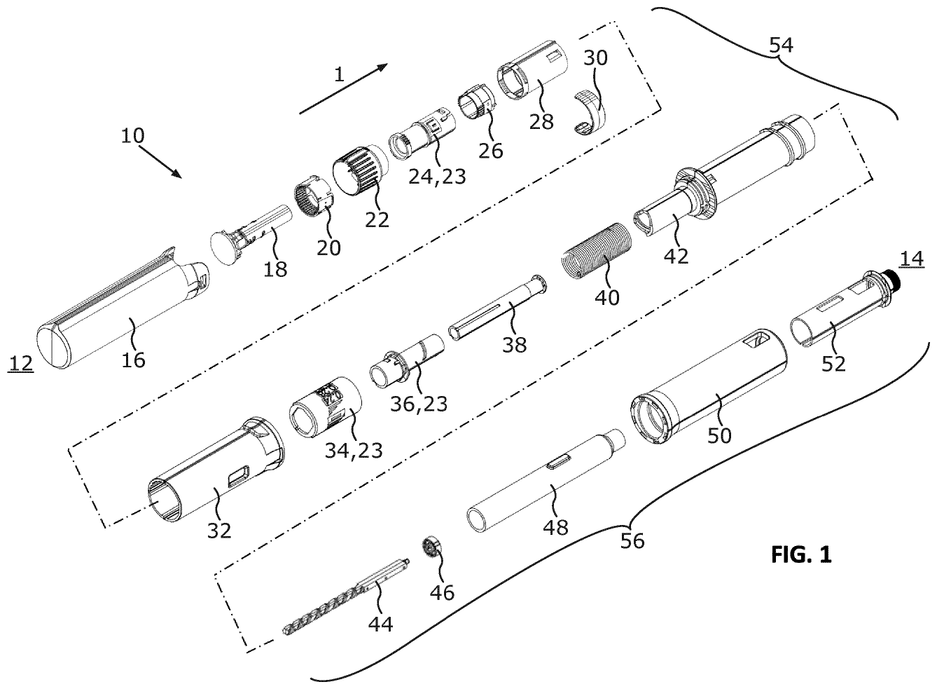

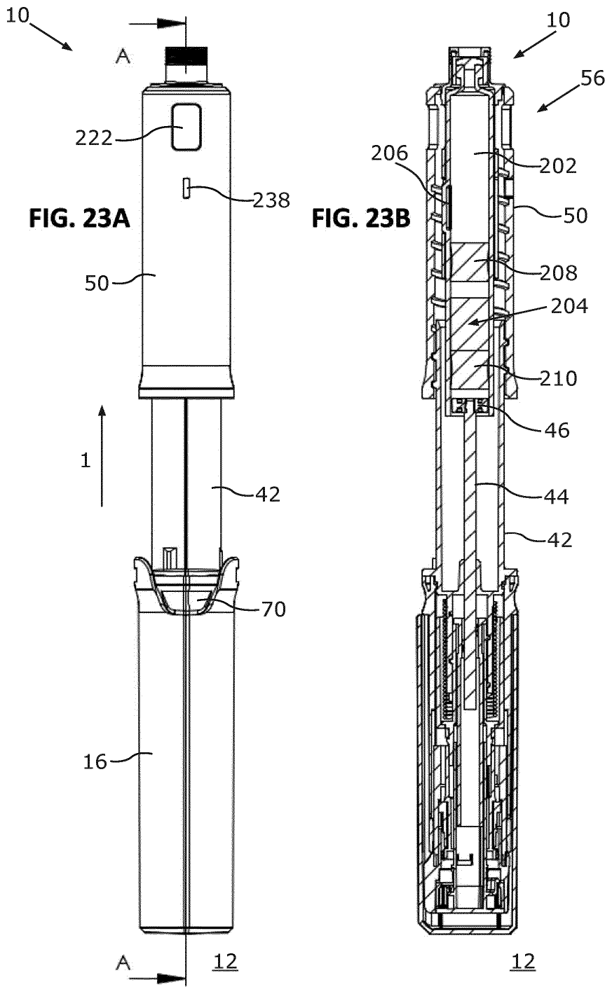

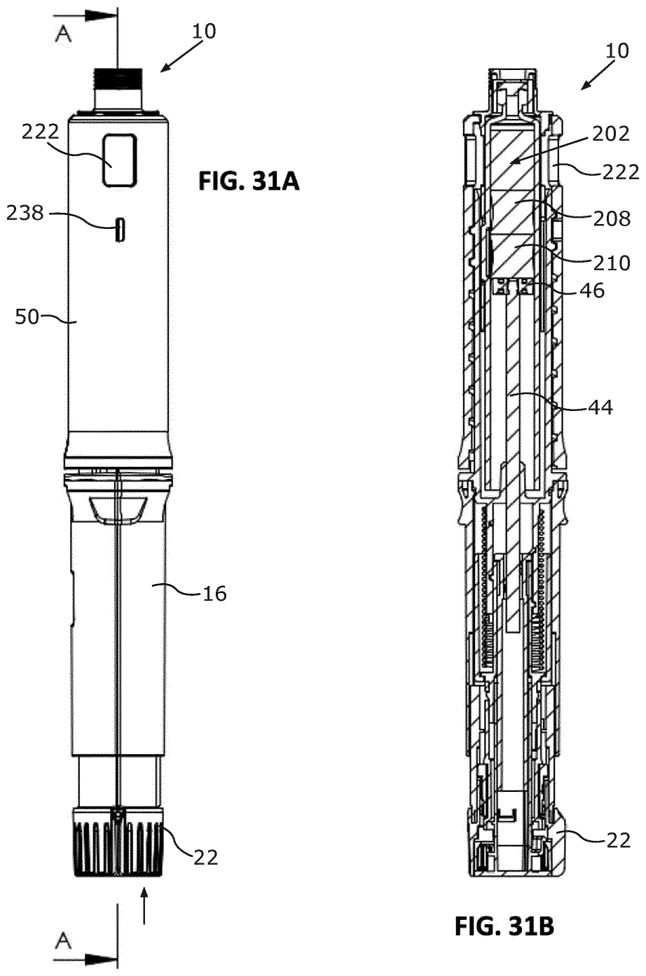

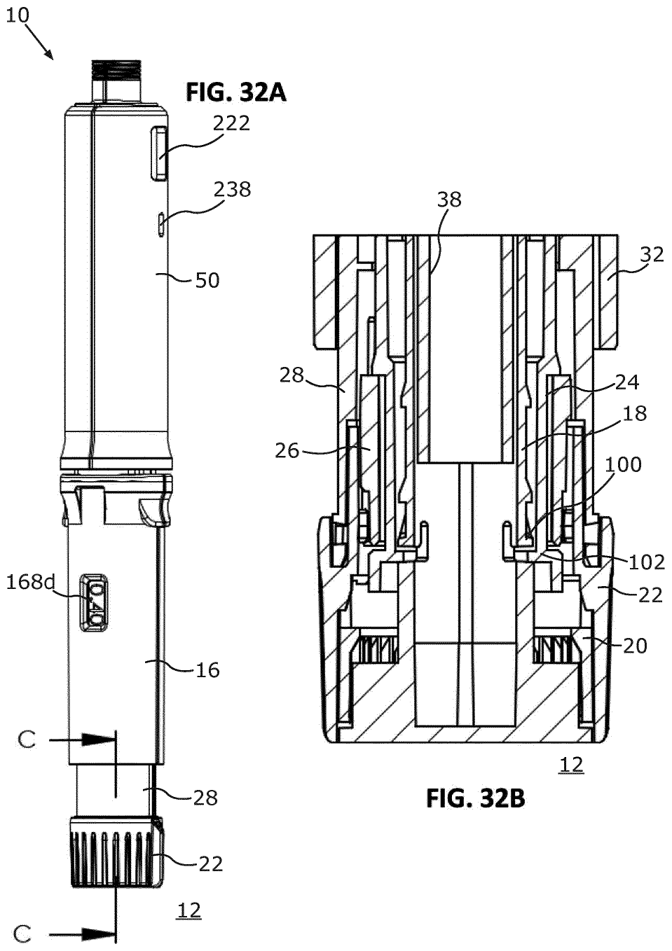

With reference to FIGS. 1 to 22B, parts of an injection pen 10 according to the disclosure are described. Afterwards, with reference to FIGS. 23A to 33B it is described how the pen is meant to be used.

FIG. 1 shows an exploded view of a medicament delivery device in form of an injection pen 10. The injection pen 10 comprises—in an order from a distal end 12 to a proximal end 14—a knob cover 16 that can also be called knob lock, cover or holding element, an injection button 18 that can be part of an actuation member, a snap ring 20, a dose setting knob 22 that can also be called dose setting element, dose adjusting member or knob and can be part of an actuation member, a snap element 24 that can also be called dose setting device, a connector 26, a dose selector 28, a knob key 30 that can also be called a clip, a housing 32 that can also be called body, a dose setting sleeve 34 that can also be called dose sleeve or dose indication member, a driver 36, a nut 38, a spring 40, a piston rod guide 42 that can also be called piston guide, a piston rod 44, a piston disc 46 that can also be called a bearing, a dual chamber cartridge 48 that can also be called a medicament container, a fluid compartment or a cartridge, a cartridge container 50, and a cartridge holder or cartridge key 52. The assembly of the cartridge container 50 and the cartridge holder 52 can also be called medicament container holder. Thereby, the cartridge container 50 provides an outer container holder and the cartridge key 52 provides an inner container holder of the container holder.

While the above mentioned parts of the injection pen 10 can each be formed as separate parts to simplify production of the separate parts, it would be generally possible to form one or more of the parts integrally with each other. For example, the injection button 18, the snap ring 20, and/or the dose setting knob 22 could be formed integrally with each other. Furthermore, the dose setting sleeve 34 and the driver 36 could be formed integrally with each other. Generally, even the housing 32 and the piston rod guide 42 could be formed integrally with each other.

The different parts can be grouped together to define different functional units. E.g. the section between the injection button 18 and the piston rod guide 42 can be called a dose setting mechanism 54, a dose setting unit, a dose delivery mechanism and/or a dose delivery activation mechanism. On the other hand, the section between the piston rod guide 42 and the cartridge key 52 can be called drug reconstitution unit 56 or reconstitution means. The cartridge container 50 and the cartridge holder 52 can be called a cartridge holding unit. The cartridge container 50, the cartridge holder 52, and the cartridge 48 can be called a cartridge unit. For example, the cartridge unit can be sold—in a preassembled state or as separate parts—separately from the rest of the injection pen 10.

Next, the above-mentioned parts of the injection pen 10 are described in the order starting from the distal end 12 and ending at the proximal end 14, the distal end 12 and the proximal end 14 being opposite ends of the injection pen 10 and the proximal end 14 comprising a dispensing outlet:

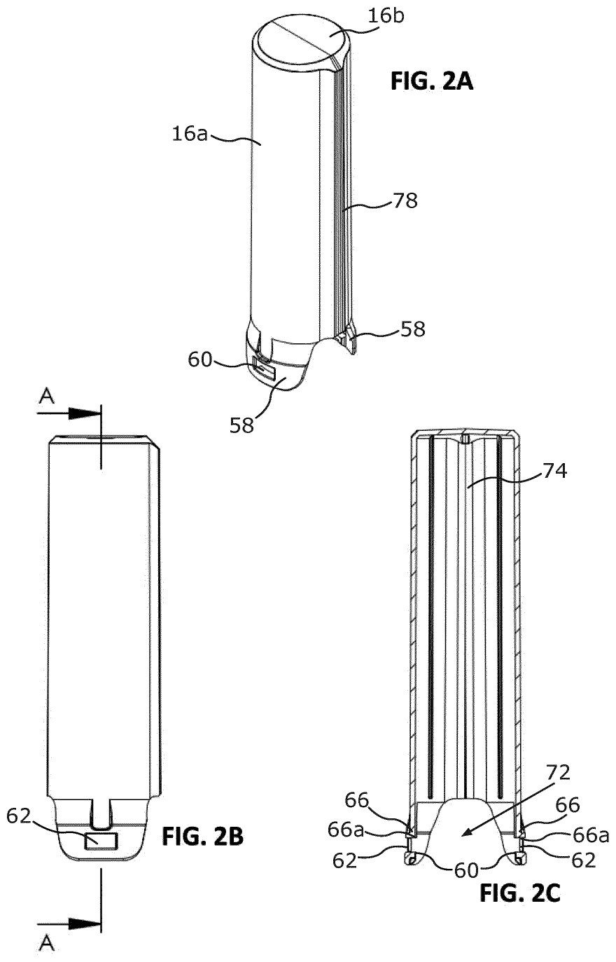

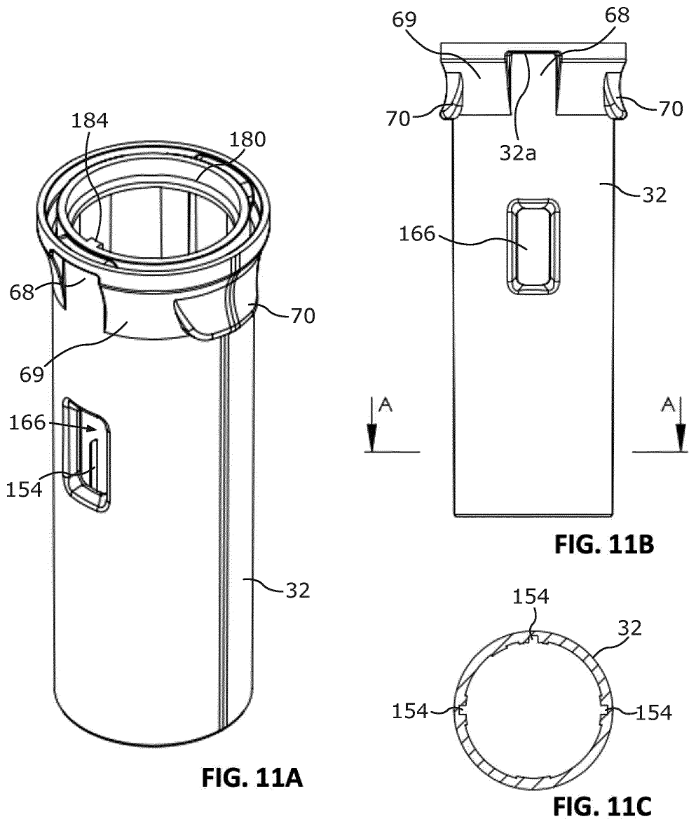

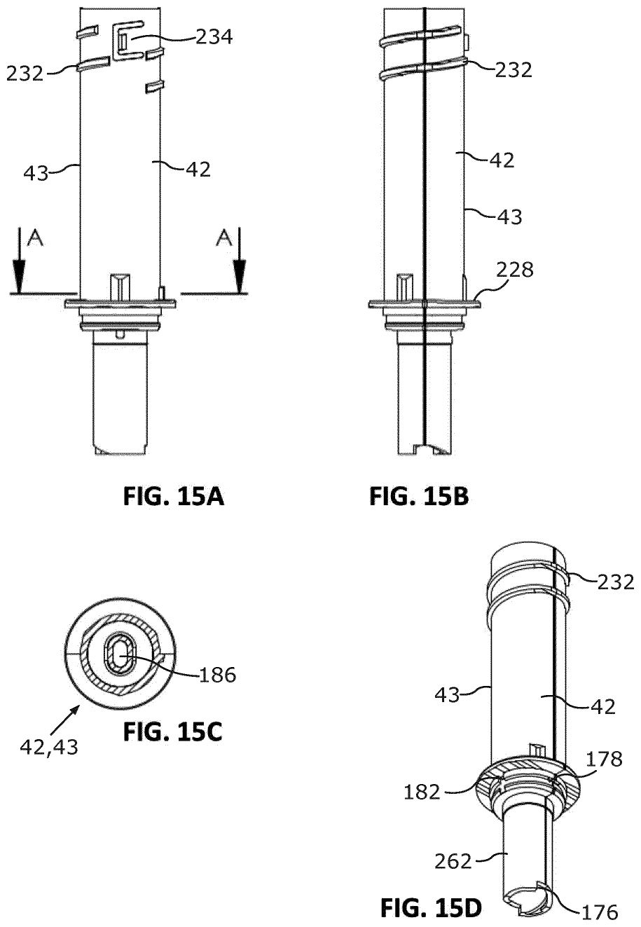

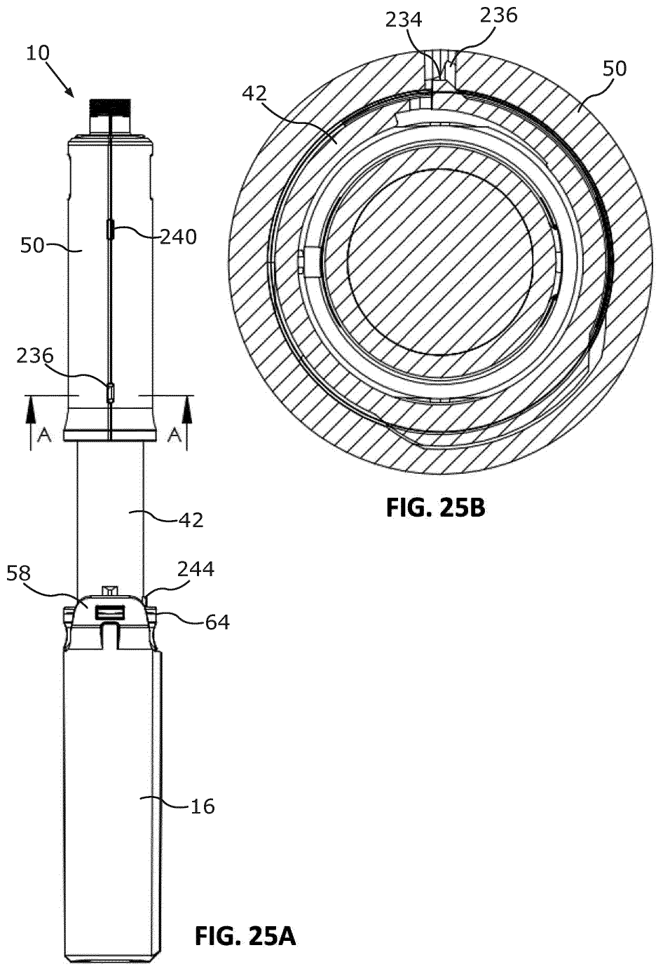

FIGS. 2A to 2C depict the knob cover 16. The knob cover 16 covers the dose setting knob 22 during delivery, i.e. shipping, of the injection pen 10 to a costumer, e.g. the patient. The knob cover 16 is fully detachable from the rest of the injection pen 10. The knob cover 16 is attachable to the housing 32 and/or detachable from the housing 32 via two deformable wings 58 that can be deflected outwardly, i.e. in a radial direction, to detach the knob cover 16 from the housing 32. The wings 58 form a proximal end section of the knob cover 16. On an inner surface of each of the wings 58, form-fitting engagement means in the form of a lug 60 are provided, that are configured to engage with the housing 32, in particular with a radially extending coupling surface 228 (cf. FIG. 15C) formed on the piston guide 42, to axially fixate the knob cover 16 relative to the housing 32 in a distal direction. Next to each of the lugs 60, one window 62, i.e. a radially extending opening, is formed in the wings 58. When the knob cover 16 is attached to the housing 32, the windows 62 are positioned at an axial position where the housing 32 forms a circumferentially extending elevation 64 (cf. FIG. 25A). On the distal side of each window 62, i.e. away from the lugs 60, on the inner side surface of the respective wing 58, an abutment 66 is formed. The abutment 66 has a width that is adapted to a width of a recess or cut-out 68 (cf. FIG. 11A) on an outer surface of the housing 32, more precisely in a chamfered portion 69 formed on the outer surface of the housing 32. Furthermore, the abutment 66 forms a front surface 66a that axially abuts a radially extending surface 32a (cf. FIG. 11B) defining a proximal end of the cut-out 68 when the knob cover 16 is attached to the housing 32. The radially extending surface 32a defines a stop surface that stops proximal movement of the knob cover 16 relatively to the housing 32, e.g. if the injection pen 10 is dropped onto a floor with the distal end 12 first. In order to further ensure that the knob cover 16 does not move past its attached position in the proximal direction 1, axial abutment elevations 70 (cf. FIG. 11A) can be formed on the outer surface of the housing 32. The elevations 70 are configured to engage with clearances 72 (cf. FIG. 2C) formed between the wings 58 so that proximal front surfaces of the knob cover 16 abut distal front faces of the axial abutment elevations 70.

A form-fitting engagement between the abutments 66 and the cut-outs 68 and/or a formfitting engagement between the elevations 70 and the clearances 72 make sure that the knob cover 16 is rotationally constrained relative to the housing 32 when the knob cover 16 is attached to the housing 32.

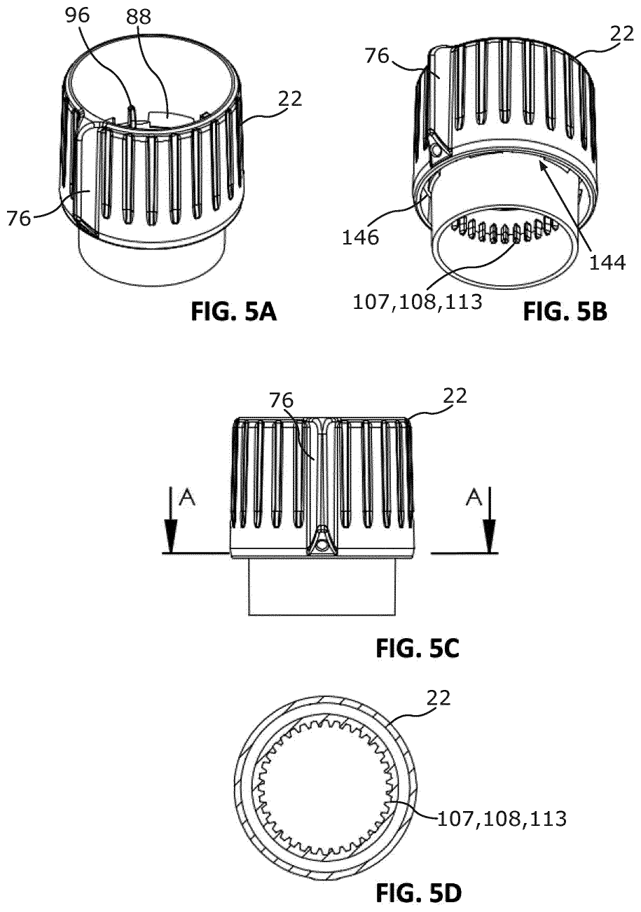

As can be seen from FIG. 2A, the knob cover 16 is only detachable from the rest of the injection pen 10 by moving the knob cover 16 linearly in a distal direction. In order to do so, a linear recess 74 is formed on the inner circumferential surface of the knob cover 16 that corresponds to an anti-rolling means 76 (cf. FIG. 5B) of the dose setting knob 22 in the form of an axially extending rib. Therefore, the dose setting knob 22 is blocked from rotating inside the knob cover 16 by the form-fitting engagement of the linear recess 74 and the anti-rolling means 76. The knob cover 16, as can be seen in FIG. 2A, also forms anti-rolling means 78 in form of an axially extending rib on the outer surface of the knob cover 16. The anti-rolling means 76 and 78 make sure that the injection pen 10 and the knob cover 16 do not roll away when placed on a flat surface. As can be also seen from FIG. 2A, the knob cover 16 has a closed circumference 16a and a closed face 16b at its distal end. Therefore, the knob cover 16 forms a closed sleeve around the distal section of the injection pen 10.

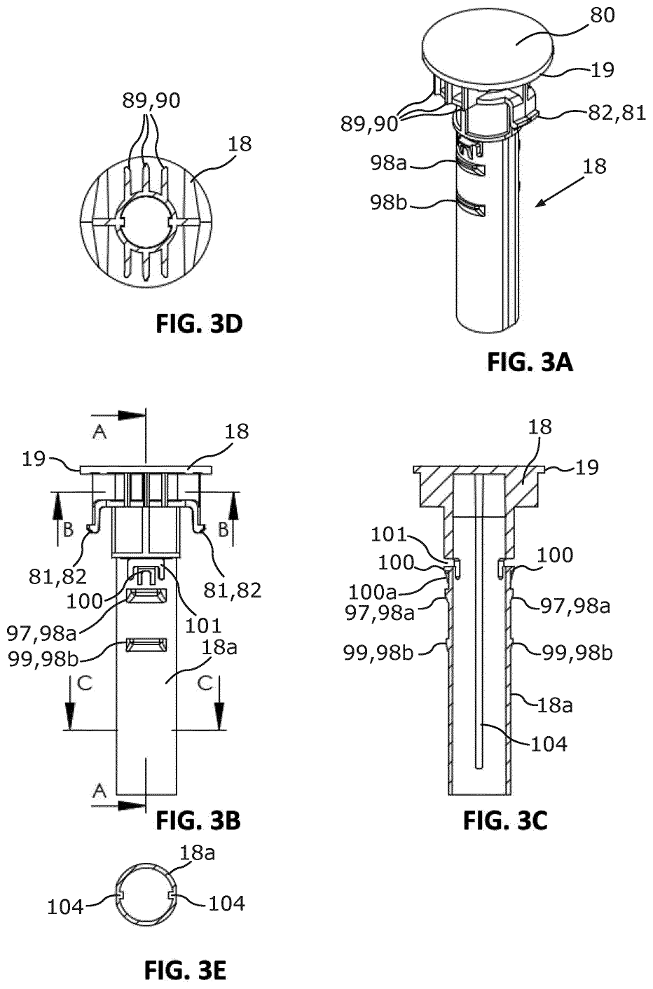

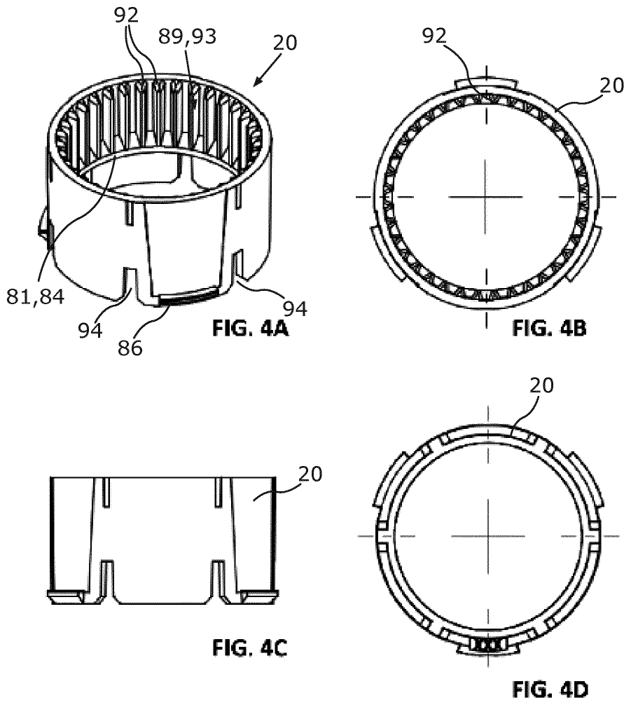

FIGS. 3A to 3E depict the injection button 18. The injection button 18 forms a distal front surface 80 to apply a force to the injection button 18 to inject a set dose. The injection button 18 comprises an axial fixation means (element) 82 to axially attach the injection button 18 to the snap ring 20 (cf. FIG. 4A-4D) which is axially connected to the dose setting knob 22 (cf. FIG. 5A to 5D). The axial fixation means 82 comprise two elastically deformable hooks 82 which engage with a circumferentially extending rib 84 on the snap ring 20. The snap ring 20 also comprises an axial fixation means (element) 86 in the form of elastically deformable bendable hooks that engage with an undercut 88 formed in the dose setting knob 22. The injection button 18, the snap ring 20 and the dose setting knob 22 are permanently axially fixed to each other in an assembled state of the dose delivery mechanism 54.

The injection button 18 also forms rotation fixation means 90 in the form of radially extending ribs. The ribs 90 are form-fittingly engaged with rotation fixation means 92 (cf. FIG. 4A) in the form of teeth arranged in an inner circumferential surface of the snap ring 20 to rotationally connect the injection button 18 to the snap ring 20. The rotation fixation means 92 form a toothed part 93 of the snap ring 20 and the ribs 90 form an engaging part of the injection button 18. The snap ring 20 comprises rotation fixation means 94 in the form of axially extending recesses that define side surfaces of the elastically deformable bendable hooks 86 and that engage with rotation fixation means 96 in the form of axially extending ribs (cf. FIG. 5A) on the inner circumferential surface of the dose setting knob 22.

After assembly and in an assembled state of the dose delivery mechanism 54, the injection button 18, the snap ring 20 and the dose setting knob 22 are rigidly connected with each other and form both a dose setting member and an actuation member of the dose delivery mechanism 54.

The injection button 18 forms a cylindrical portion 18a. On the cylindrical portion 18a, assembling means 98 in the form of elevations are formed to axially preassemble the injection button 18 with the snap element 24. More precisely, the lower, i.e. proximal, assembling means 98b (cf. FIG. 3C) restricts distal movement of the injection button 18 relative to the snap element 24 by interfering with coupling means 102 on the snap element 24. The upper, i.e. distal, assembling means 98a restricts proximal movement of the injection button 18 relative to the snap element 24 by interfering with coupling means 102 on the snap element 24 after pre-assembly and distal movement of the injection button 18 after final assembly. When the snap element 24 and the injection button 18 are preassembled, i.e. in a preassembled state, the coupling means 102 is arranged between the proximal assembling means 98b and the distal assembling means 98a. In the preassembled state, the injection button 18 is not yet rigidly connected to the snap ring 20 and the dose setting knob 22. However, when the coupling means 102 is arranged distally from the distal assembling means 98a, i.e. in the assembled state, the injection button 18 is rigidly connected to the snap ring 20 and the dose setting knob 22. The injection button 18 also forms coupling means (element or elements) 100 in the form of protrusions being arranged on an outer circumferential surface of the injection button 18 on elastically inwardly bendable portions. The inwardly bendable portions extend in an axial direction and are sectionally surrounded by cut-outs 101.

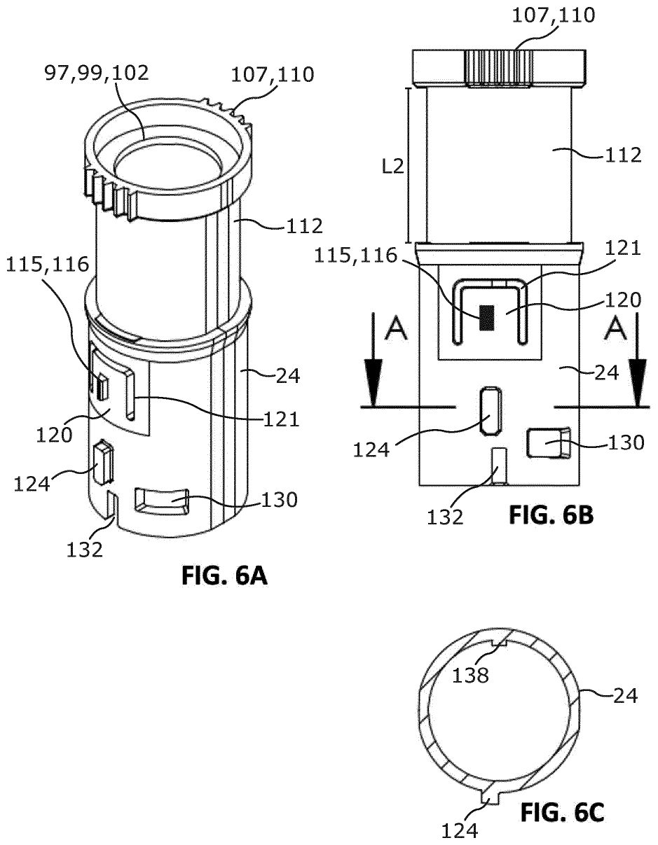

The coupling means 100 are configured to permanently axially lock the injection button 18 and therefore also the snap ring 20 and the dose setting knob 22 to the snap element 24 after the injection has been completed to render the injection pen 10 inoperable. Namely, when the injection button 18 is moved axially to initiate the dose delivery, the coupling means 100 pass the radially inwardly extending coupling means in the form of a circumferentially extending ledge 102 (cf. FIG. 6A) on the snap element 24. The radially inwardly extending ledge 102 causes the protrusions being arranged on elastically inwardly bendable portions 100 to bend inwardly until the protrusions have passed the ledge 102. In order to reduce the force needed to push the protrusions 100 past the ledge 102, the protrusions 100 form chamfered outer surfaces 100a. Alternatively or additionally, the ledge 102 could form a chamfered inner surface. When the protrusions 100 have passed the ledge 102, they snap back into their neutral position which causes the injection button 18 to be permanently axially locked relative to the snap element 24. This feature makes sure that the injection pen 10 can only be used one single time to inject exactly one dose.

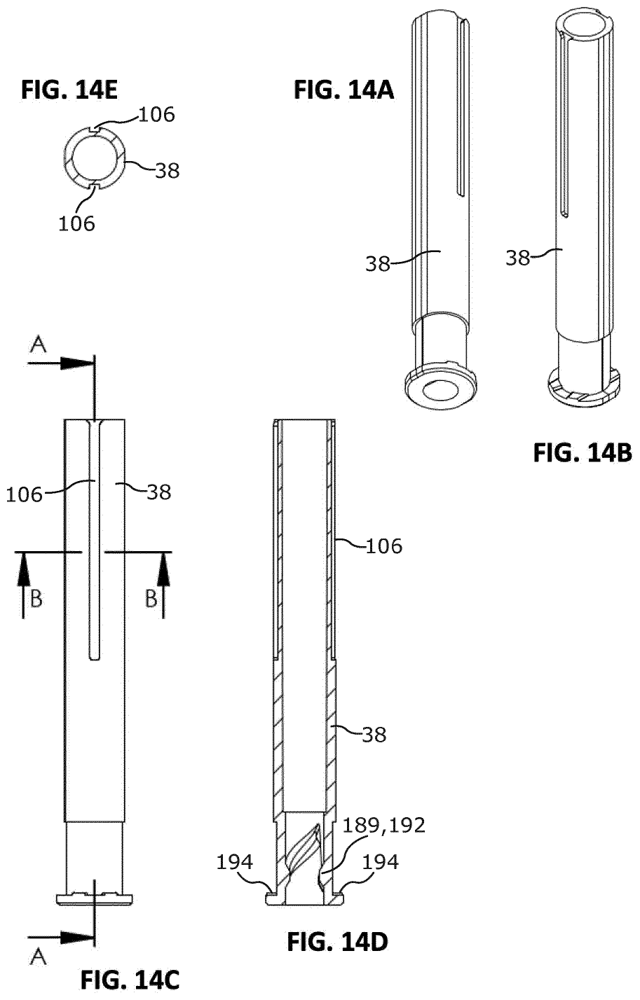

As can be best seen in FIG. 3C and 3E, the injection button 18 comprises an axially extending rib 104 on its inner circumferential surface. The axially extending rib 104 engages in an axially extending groove 106 of the nut 38 (cf. FIG. 14C) to form rotation fixation means. Due to the axially extending rib 104 and the corresponding axially extending groove 106, the injection button 18 and the nut 38 can move axially relative to each other but are rotationally fixed to each other.

As can be best seen on FIG. 5B and 5D, a set of teeth 108 are formed in an axial section on an inner circumferential side of the dose setting knob 22. These teeth 108 are configured to mesh with a set of teeth 110 arranged in a distal section on an outer circumferential surface of the snap element 24 (cf. FIG. 6A-6C) during dose setting. Therefore, when the dose setting knob 22 is rotated during dose setting, the snap element 24 is rotated too. As can be seen in FIG. 6A, the teeth 110 are arranged only in two opposite sections of the snap element 24 while sections between the two opposite sections do not form any teeth. This has advantages regarding production of the injection pen 10. Alternatively, the teeth 110 could be formed along the whole circumference of the snap element 24.

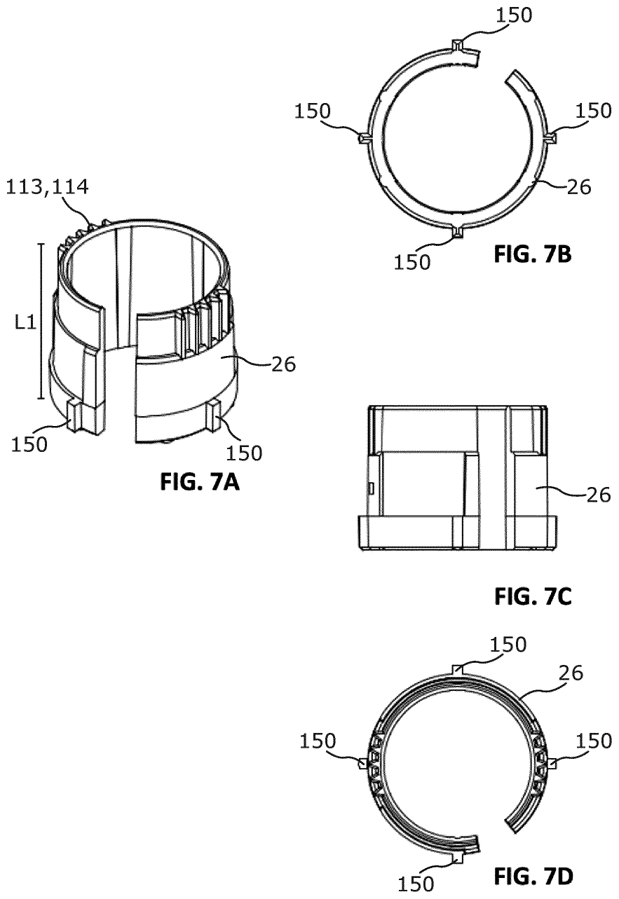

The snap element 24 forms an axial section with a reduced cross section forming a coupling surface 112 for the connector 26. The connector 26 shown in the figures is formed as an integral part. However, the connector 26 could also be formed from multiple, e.g. two, parts connected to each other, e.g. via a form-fitting connection. The connector 26 has an open cross section (cf. FIG. 7D) so it is clippable onto the snap element 24 at the reduced cross section. The connector 26 is axially fixedly connected to the snap element 24 in both directions due to the connector 26 having a length L1 in the axial direction that corresponds to a length L2 in the axial direction of the axial section with the reduced cross section. However, the connector 26 is rotatable relative to the snap element 24. When the dose setting knob 22 is pushed in the proximal direction 1 to initiate dose delivery, the teeth 108 of the dose setting knob 22 engage with a set of teeth 114 formed on an outer circumferential surface of the connector 26 instead of the teeth 110 of the snap element 24 so that the snap element 24 can rotate relative to the dose setting knob 22 during dose delivery. The engagement between the teeth 108 of the dose setting knob 22 and the teeth 114 of the connector 26 makes sure that the dose setting knob 22 does not rotate during dose delivery with respect to the housing 32 due to connector 26 being rotationally fixed to the housing 32 via the dose selector 28.

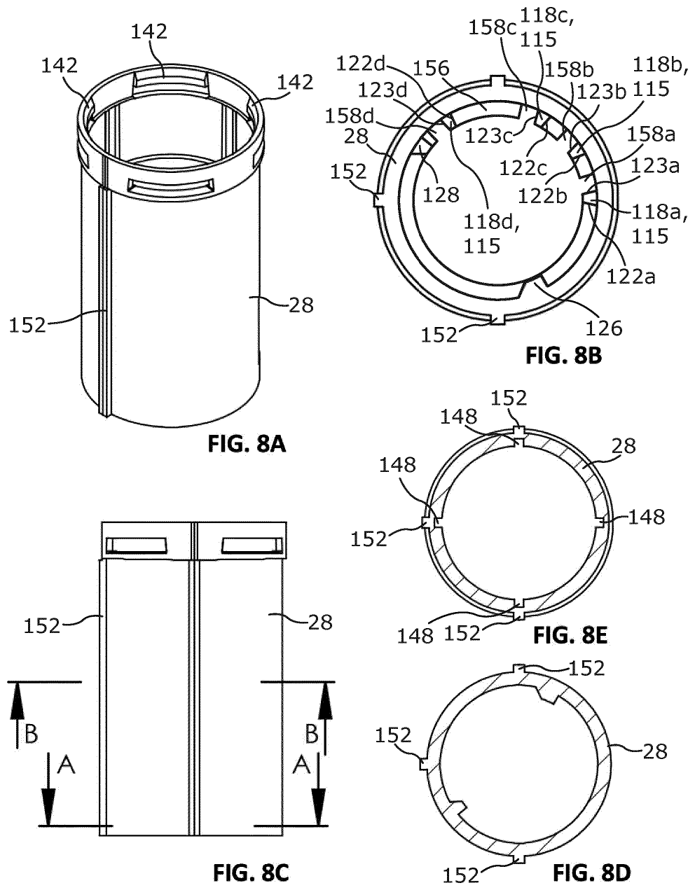

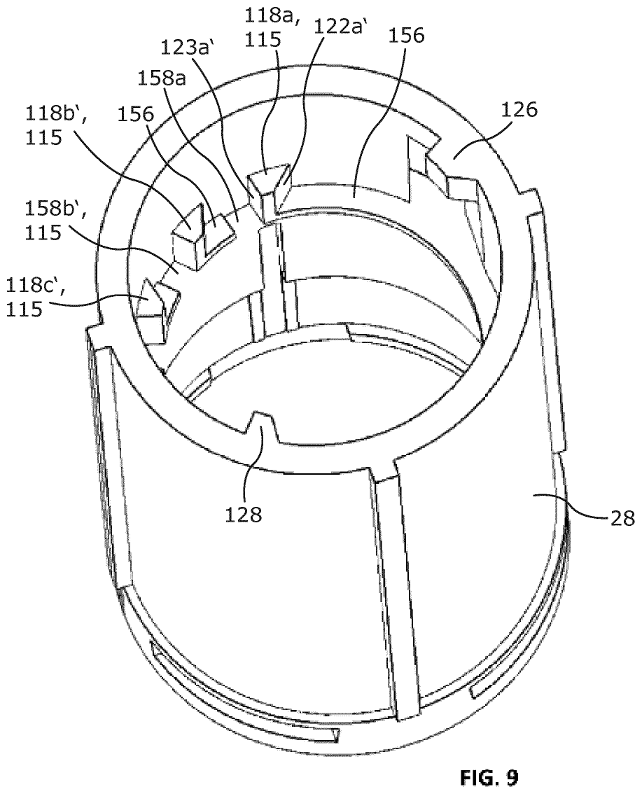

The snap element 24 further comprises an engagement feature 116 in the form of an axially extending radial projection. The engagement feature 116 is an axially extending rib. The engagement feature 116 can have a symmetrical cross section in a radial plane perpendicular to a longitudinal axis of the injection pen 10 or an asymmetrical cross section. The engagement feature 116 is configured to engage with dose stops 118a, 118b, 118c, and 118d (cf. FIG. 8B) formed on an inner circumferential surface of the dose selector 28 to set a desired dose. Therefore, the engagement feature 116 is used as a dose definition element and the engagement feature 116 together with the dose stops 118a, 118b, 118c, 118d form a dose definition mechanism 115 of the injection pen 10. The dose definition element 116 is located on an elastically deformable section 120, i.e. an axially extending arm partially surrounded by a cut-out 121. The elastically deformable section 120 bends inwardly when the dose definition element 116 passes one of the dose stops 118a, 118b, 118c, and 118d. In order to reduce the force needed to rotate the dose setting knob 22 and the snap element 24 relative to the dose selector 28 to enlarge or decrease the set dose, the dose stops 118 have chamfered side surfaces 122a-d and 123a-d. According to the embodiment shown in FIG. 8B, the dose stops 118a-d have a symmetrical cross section in the radial plane perpendicular to the longitudinal axis of the injection pen 10. In other words, the chamfered side surfaces 122 and 123 have pitches that are equal to each other regarding their amount. According to another embodiment shown in FIG. 9, chamfered side surfaces 122a′-d′ that get in contact with the projection 116 to deform the elastically deformable section 120 when the dose is set to a higher dose have a smaller pitch than chamfered side surfaces 123a′-d′ that get in contact with the projection 116 when the dose is set to a lower dose. The side surfaces 123a-d define rotational positions corresponding to settable doses. The spring 40 is configured to rotate the snap element 24 relative to the dose selector 28 so that the dose definition element 116 abuts one of the side surfaces 123a-d.

The snap element 24 further comprises a hard stop 124 in the form of an axially extending rib that abuts a hard stop 126 formed on the dose selector 28 when the injection pen 10 is delivered to a costumer. The hard stop 126, contrary to known pens, does not correspond to a zero-dose stop but instead corresponds to a pre-set dose stop. A further discussion regarding this feature follows. The hard stop 124 is axially distanced from the dose definition element 116 but axially aligned with the dose definition element 116. The hard stop 124 is configured to abut an end of dose setting hard stop 128.

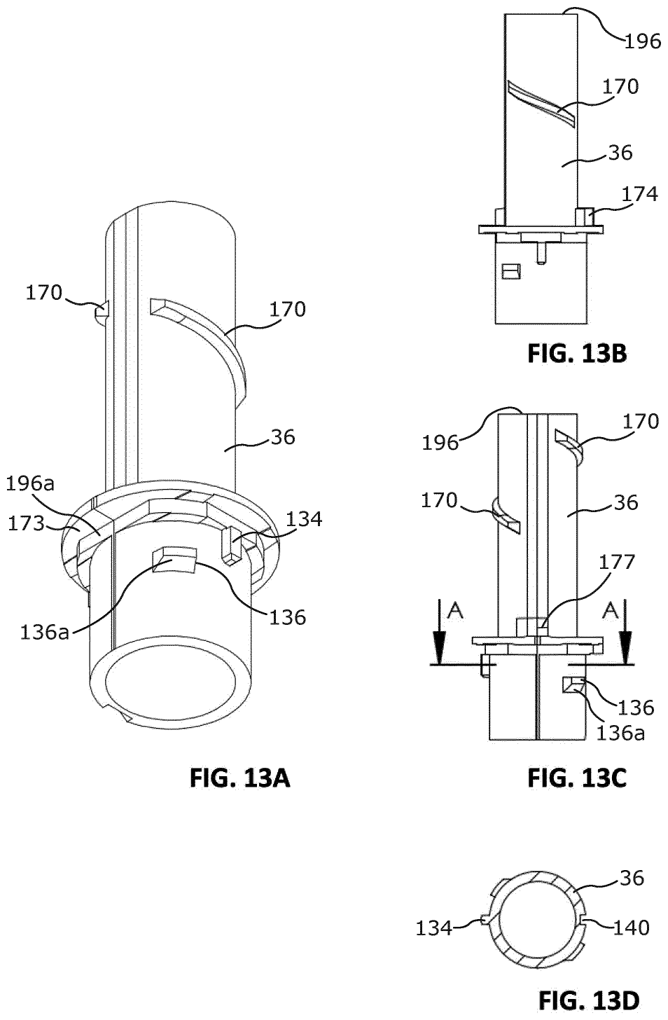

The snap element 24 further comprises axial and rotational fixation means in the form of a radially extending opening 130 and an axially extending slot 132 to axially and rotationally fix the snap element 24 to the driver 36. As can be seen in FIG. 13A, the driver 36 has an axially extending rib 134 that is configured to engage with the slot 132 of the snap element 24. Furthermore, the driver 36 has a protrusion 136 with a chamfered surface 136a that engages with the opening 130 of the snap element 24. While the opening 130 and the protrusion 136 form the axial fixation means, the slot 132 and the rib 134 form the rotational fixation means. Due to the axial and rotational fixation means, the snap element 24 and the driver 36 can be connected to each other in one defined relative rotational position. In order to strengthen the rotational fixation between the snap element 24 and the driver 36, an axially extending rib 138 is formed on an inner circumferential surface of the snap element 24 (cf. FIG. 6C) that engages with an axially extending groove 140 (cf. FIG. 13D) on an outer circumferential surface of the driver 36.

FIGS. 8A to 8E depict the dose selector 28. The dose selector 28 comprises an axial fixation means (element) 142 in the form of circumferentially extending projections on an inner circumferential surface of a distal section of the dose selector 28. The dose selector 28 is axially fixed to the dose setting knob 22 by inserting the distal section with the axial fixation means 142 into a circumferentially extending intake 144 (cf. FIG. 5B). In the intake 144, the dose setting knob 22 forms an axial fixation means (element) 146 in the form of circumferentially extending protrusions on an outer circumferential surface which get engaged with the an axial fixation means (element) 142 of the dose selector 28 to form an axial connection that allows relative rotational movement between the dose selector 28 and the dose setting knob 22.

As can be seen best on FIG. 8E, rotation fixation means 148 in the form of axially extending grooves are formed on an inner circumferential surface of the dose selector 28. The rotation fixation means 148 are engaged with rotation fixation means 150 in the form of axially extending ribs formed on the outer circumferential surface of the connector 26 (cf. FIG. 7B). The rotation fixation means 148, 150 allow axial movement between the dose selector 28 and the connector 26. The dose selector 28 further comprises rotation fixation means 152 in the form of axially extending ribs formed on an outer circumferential surface of the dose selector 26. The rotation fixation means 152 engage with rotation fixation means 154 in the form of axially extending grooves formed on the inner circumferential surface of the housing 32 (cf. FIG. 11C). The rotation fixation means 152, 154 are configured to define one single possible rotational alignment that allows insertion of the dose selector 28 into the housing 32. The rotation fixation means 150, 152 allow axial movement between dose selector 28 and the housing 32.

In order to define deliverable doses, the dose selector 28 (cf. FIG. 8B) forms a circumferentially extending rib 156 with cut-outs 158a, 158b, 158c, and 158d. The cut-outs 158a, 158b, 158c, and 158d are assigned to the respective dose stops 118a, 118b, 118c, and 118d. The rib 156 with its cut-outs 158a, 158b, 158c, and 158d makes sure, that injection is only possible if the dose definition element 116 of the snap element 24 is at an angular position relating to one of the cut-outs 158a, 158b, 158c, and 158d, i.e. relating to one of the settable doses. If the dose definition element 116 is not at an angular position relating to one of the cut-outs 158a-d, axial movement of the dose definition element 116, and therefore the snap element 24, relative to the dose selector 28 is blocked by the circumferentially extending rib 156. As can be seen from FIG. 8B, there is no cutout assigned to the pre-set dose hard stop 126. Therefore, starting an injection is inhibited when the injection pen 10 is set to the pre-set dose.

According to an alternative embodiment, the circumferentially extending rib 156 could be arranged to interact with the hard stop 124 instead of the dose definition element 116. Therefore, injection would only be possible if the hard stop 124 of the snap element 24 would be at an angular position relating to one of the cut-outs 158a, 158b, 158c, and 158d, i.e. relating to one of the settable doses.

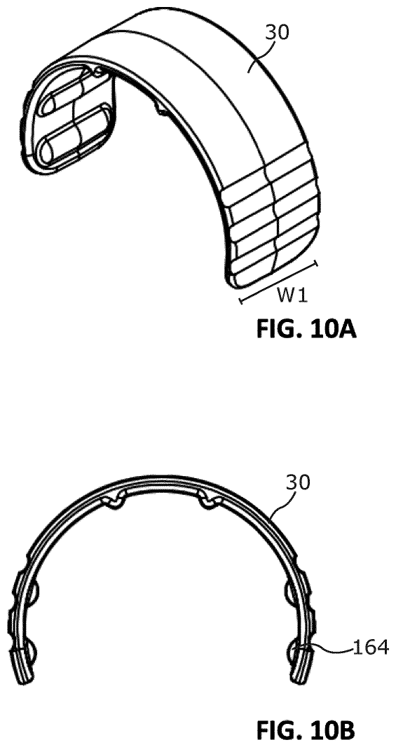

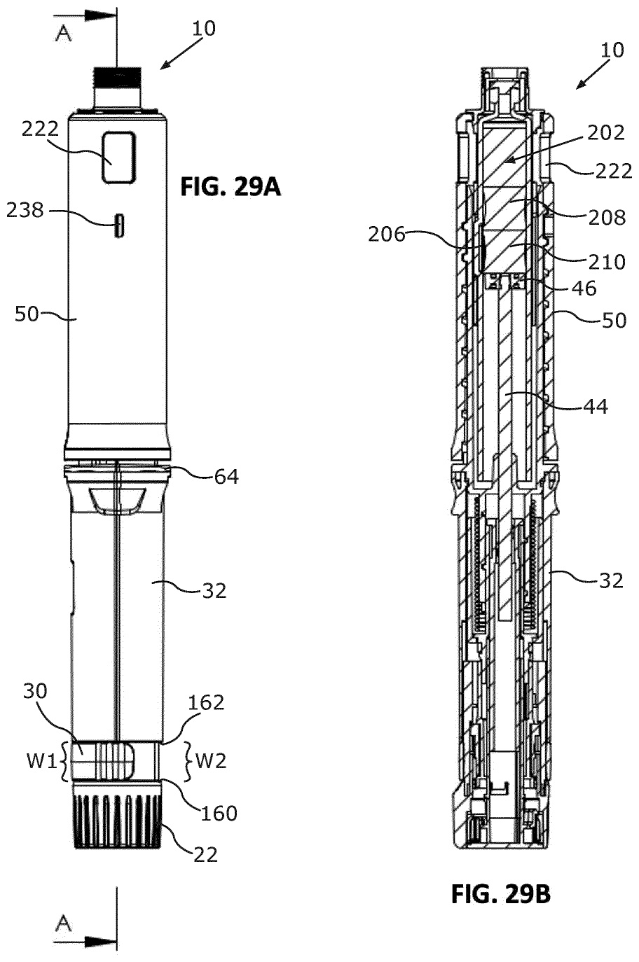

FIGS. 10A and 10B depict the knob key 30. The knob key 30 is configured to be attached to the outer circumferential surface of the dose selector 28 to keep the dose setting knob 24 from unintentionally moving in the proximal direction 1 relative to the housing 32 if the injection pen 10 in an as-delivered state drops onto its proximal end. The clip element 30 has a width W1 that corresponds to a width W2 (cf. FIG. 29A) between a proximal edge 160 of the dose setting knob 22 and a distal edge 162 of the housing 32. The knob key 30 is C-shaped and has holding protrusions 164 that interact with the rotation fixation means 152 on the outer circumferential surface of the dose selector 28 to attach the knob key 30 to the dose selector 28. The knob key 30 can be taken off the dose selector 28 by slightly bending the C-shaped knob key 30. In the as-delivered state, the knob cover 16 extends around the knob key 30 to hold the knob key 30 in place. The knob key 30 can only be taken off the dose selector 28 after the knob cover 16 has be removed.

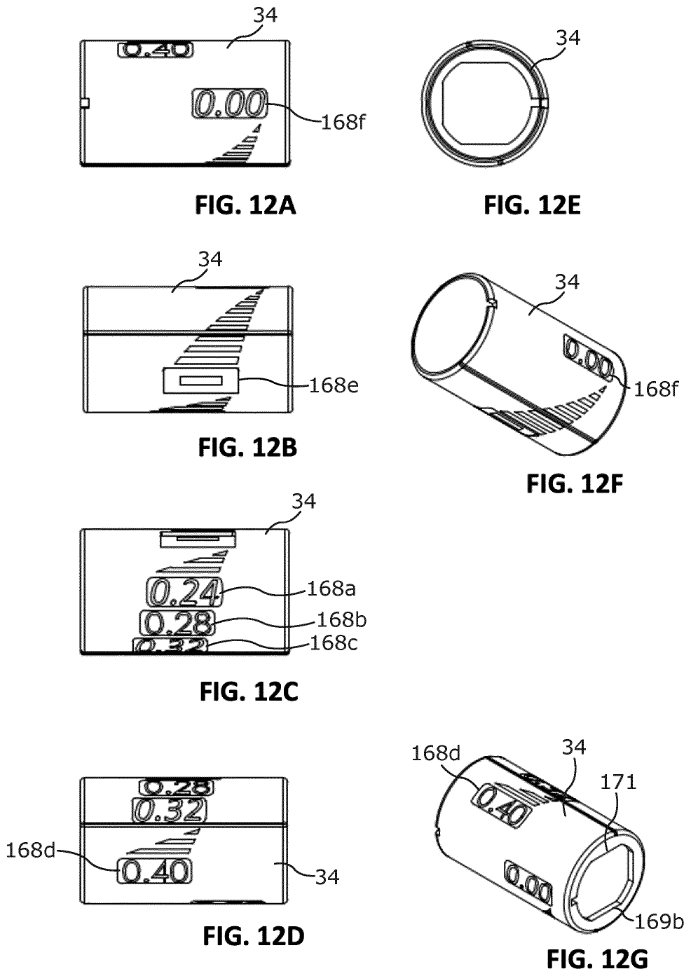

The housing 32 is shown in FIGS. 11A to 11C. The housing 32 forms a viewing window 166 for displaying a state of the injection pen 10, in particular a set dose, indicated by the dose sleeve 34 through the window 166. The dose sleeve 34 rotates relative to the housing during dose setting and dose delivery which causes a change of what is displayed through the window 166. In different circumferential positions along the outer circumferential surface of the dose sleeve 34, labels 168a-168d (cf. FIGS. 12C and 12D) for different settable doses are located. Furthermore, a preset-dose label 168e (cf. FIG. 12B) is located on the dose sleeve 34 that corresponds to a pre-set dose, i.e. an amount of medicament that would be injected if the injection could be started from the pre-set dose. As can be seen from comparing FIGS. 12A and 12B, the pre-set dose label 168e differs from a zero-dose label 168f, i.e. the label that shows that no medicament would be injected if the injection would be started in that state. This zero-dose label 168f is shown through the window 166 when the injection has been completed. The labels 168a, 168b, 168c, and 168d correspond to the settable doses defined by the dose stops 118a, 118b, 118c, and 118d.

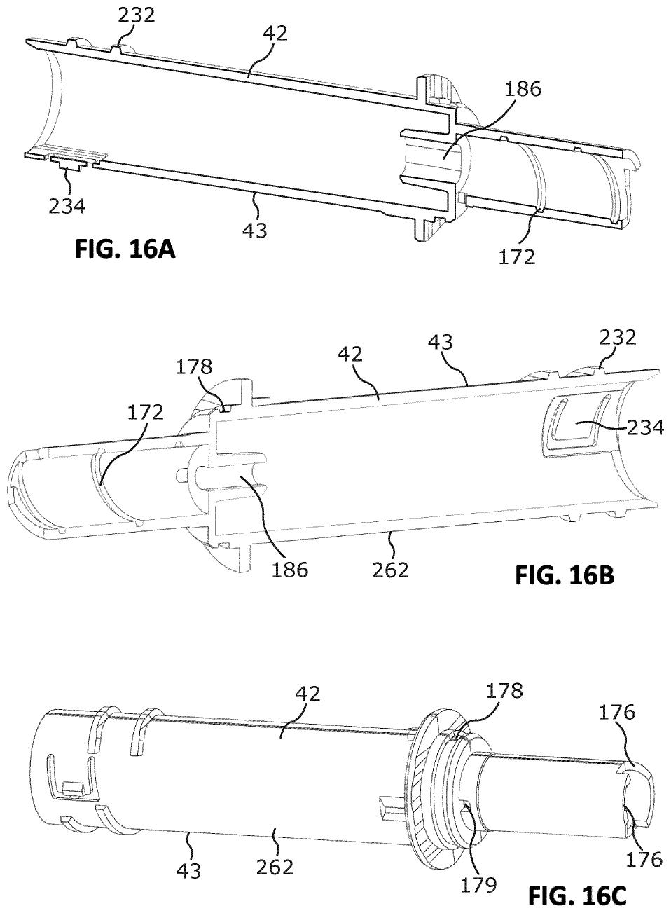

The dose sleeve 34 is rotationally and axially rigidly coupled to the driver 36 (cf. FIG. 13A-13D). In order to rotationally couple the dose sleeve 34 to the driver 36 corresponding out-of-round outer and inner circumferential surfaces 169a and 169b are formed on the driver 36 and the dose sleeve 34, respectively. Furthermore, the dose sleeve 34 forms a fixing section 171 that is pinched between a proximal end of the snap element 24 and a face surface 173 (cf. FIG. 13A) of the driver 36 to axially fix the dose sleeve 34 to the driver 36 and the snap element 24. The driver 36 forms an outer thread 170 that engages with an inner thread 172 (cf. FIGS. 16A-16B) of the piston guide 42. The threaded connection 170, 172 causes the driver 36 to rotate when the driver 36 is moved axially relative to the piston guide 42 and causes the driver 36 to move axially relative to the piston guide 42 when the driver 36 is rotated relative to the piston guide 42. Furthermore, the driver 36 defines end stops 174 that abut end stops 176 of the piston guide 42 at the end of the dose delivery. The surfaces defining the end stops 174, 176 are arranged in parallel to a middle axis of the injection pen and face in a radial direction. The driver 36 also forms attachment means 177 in the form of a radially extending hook for attaching one end section of the spring 40 to the driver 36. The other end section of the spring 40 is attached to attachment means 179 (cf. FIG. 16C) at the outer circumferential surface of the piston rod guide 42.

According to an alternative version, the dose sleeve and the driver can be formed as separate parts that are axially movable relative to each other but rotationally fixed to each other and both, the dose sleeve and the driver, can have a thread that is threadedly coupled to the housing. The thread of the dose sleeve and the thread of the driver can have different pitches.

The piston guide 42 is axially and radially fixed to the housing 32 and can therefore be considered part of the housing. In order to axially fix the piston guide 42 to the housing 32, an axial fixation means (element) 178 in the form of a circumferentially extending groove are formed on the piston guide 42 that engage with an axial fixation means (element) 180 (cf. FIG. 11A) in the form of a circumferentially extending rib formed on an inner circumferential surface of the housing 32. In order to rotationally fix the piston guide 42 to the housing 32, rotation fixation means 182 in the form of an axially extending groove are formed on an outer circumferential surface 262 of the piston guide 42 that engage with rotation fixation means 184 (cf. FIG. 11A) in the form of an axially extending rib formed on an inner circumferential surface of the housing 32. The axial and rotational fixation means 178, 180, 182, and 184 allow attachment of the piston rod guide 42 to the housing 32 in exactly one relative rotational position.

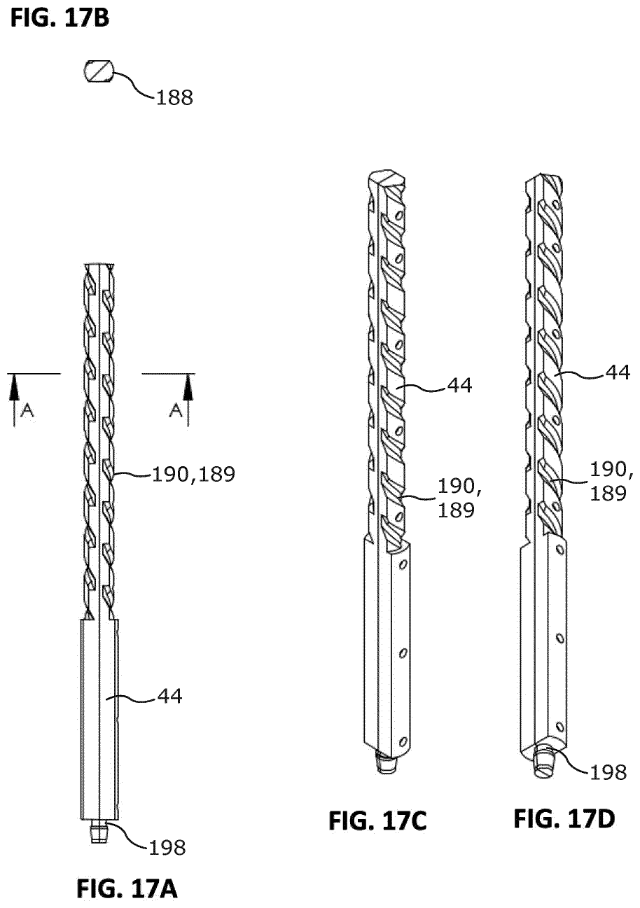

The piston guide 42 has an out of round axial opening 186 (cf. FIG. 15C) that corresponds to an out of round cross-section 188 (cf. FIG. 17B) of the piston rod 44. Therefore, the piston rod 44 is axially movable relative to the piston rod guide 42, but cannot rotate relative to the piston rod guide 42. The piston rod 44 forms an outer thread 190 that is in engagement with an inner thread 192 (cf. FIG. 14D) of the nut 38. The outer thread 190 and the inner thread 192 form a threaded connection 189 between the piston rod 44 as a first threaded element and the nut 38 at a second threaded element. The piston rod 44 and the nut 38 can move relative to each other in a compulsory guided combined axial and rotational movement. In a proximal end section of the nut 38, an annular pressing surface 194 extending in the distal direction is formed on the nut 38. This pressing surface 194 abuts a front surface 196 of the driver 36 during dose delivery. During dose delivery, the driver 36 moves in a combined axial and rotational movement relative to the piston rod guide 42 while the nut 38 is rotationally fixed to the housing 32. In order to reduce friction during dose delivery, a ball bearing and/or a glide disc made of low-friction material can be arranged between the pressing surface 194 and the front surface 196 of the driver 36. In both cases, during dose delivery, the driver 36 pushes the piston rod 44 via the nut 38 in the proximal direction 1.

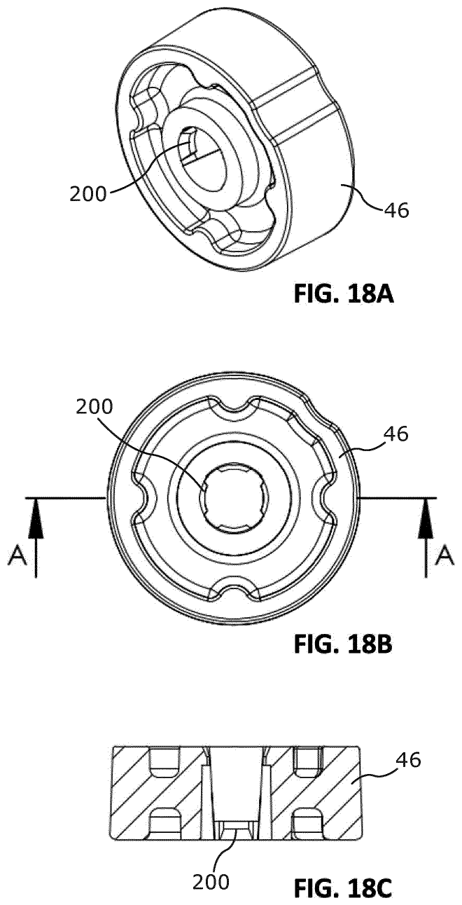

The piston rod 44, at its proximal end, forms coupling means 198 in the form of an undercut that engage with coupling means 200 in the form of radially inwardly extending ribs on an inner circumferential surface of the piston disc 46 (cf. FIG. 18A-18C).

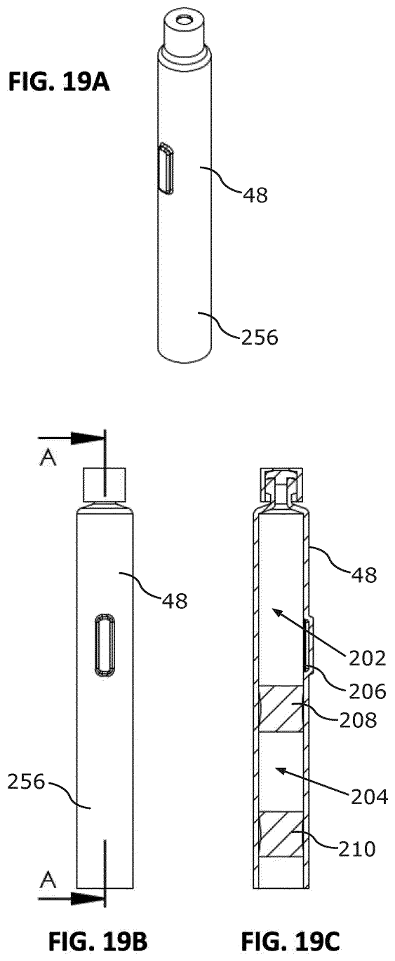

FIGS. 15A to 22B depict parts of a drug mixing or reconstitution unit 56 configured to mix different components, usually a lyophilized drug and a liquid solvent, to form an injectable liquid drug. In FIGS. 19A to 19C, the dual chamber cartridge 48 is shown. The dual chamber cartridge 48 is made of a transparent material such as glass. As can be seen from FIG. 19C, the cartridge 48 forms a first chamber 202 and a second chamber 204. In the as-delivered state shown in FIG. 19C, the first chamber 202 being arranged in proximal to the second chamber 204 comprises a bypass 206. The first chamber 202 and the second chamber 204 are separated by a first sealing element 208, e.g. made of a rubber material, that is axially slid ably arranged inside the dual chamber cartridge 48. In other words, the first sealing element 208 forms a distal end of the first chamber 202 and a proximal end of the second chamber 204. A second sealing element 210, e.g. made of a rubber material, forms a distal end of the second chamber 204. The second sealing element 210 can also be called a plunger. The piston disc 46 abuts the distal end face of the second sealing element 210 during mixture of the two components.

In the as-delivered state the lyophilized drug is in the first chamber 202 and the solvent in the second chamber 204.

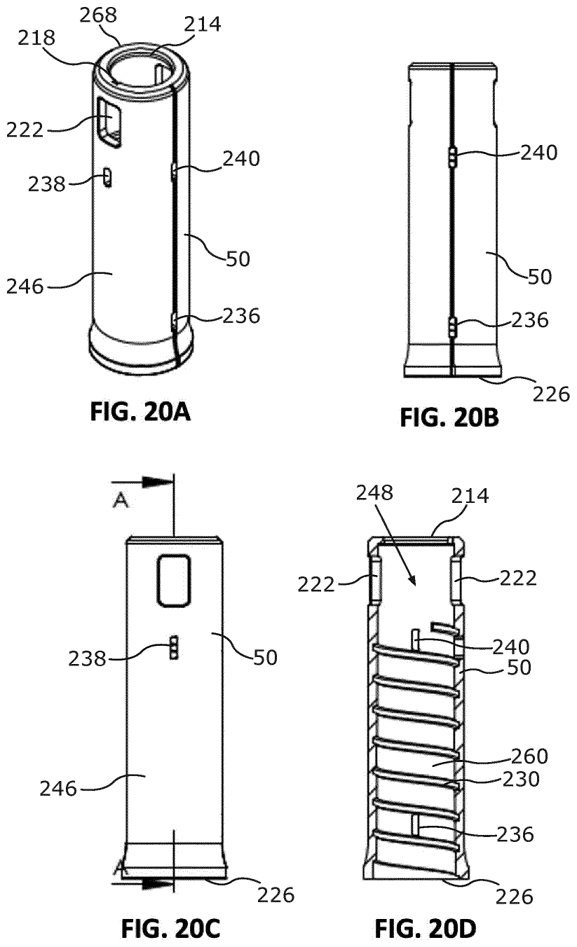

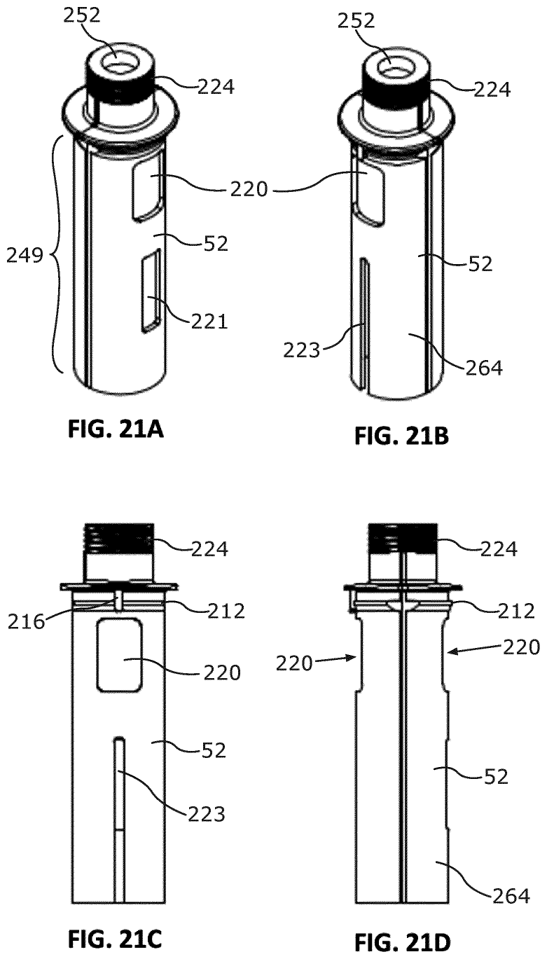

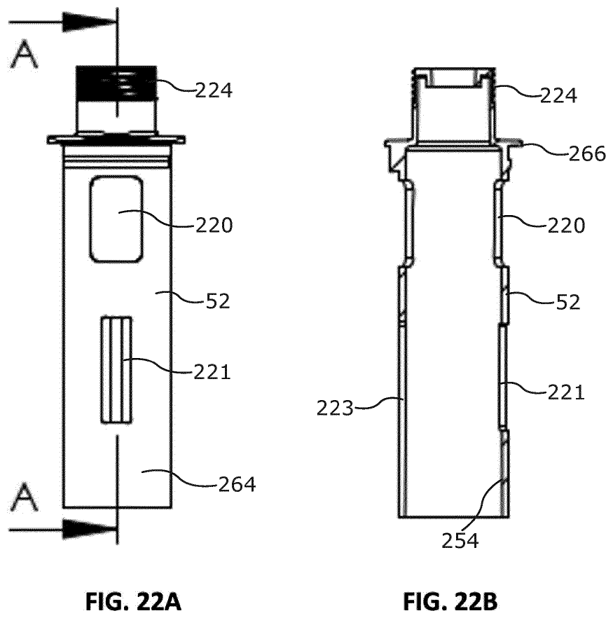

The dual chamber cartridge 48 is stored in the cartridge key 52 (cf. FIG. 21A-22b). The cartridge key 52 is axially and rotationally fixed to the cartridge container 50. To achieve that, the cartridge key 52 forms an axial fixation means (element) 212 in the form of a circumferentially extending groove that engage with an axial fixation means (element) 214 (cf. FIG. 20A) in the form of a circumferentially extending rib on an inner circumferential surface of the cartridge container 50. Furthermore, the cartridge key 52 forms a rotation fixation means (element) 216 in the form of a radially extending rib that engage rotation fixation means 218 in the form of a radially extending groove on the inner circumferential surface of the cartridge container 50. When the cartridge key 52 is attached to the cartridge container 50, an annular end face 266 (FIG. 22B) is in aerial abutment with a corresponding annular end face 268 (FIG. 20A) of the cartridge container 50. Both annular end faces 266 and 268 can be arranged perpendicular to the longitudinal axis of the pen 10. Furthermore, when the cartridge key 52 is attached to the cartridge container 50, a window 220 formed in the cartridge key 52 is aligned with a window 222 in the cartridge container 50 so that the patient can see the drug inside the transparent dual chamber cartridge 48 during reconstitution. At the proximal end of the cartridge key 52, which also defines the proximal end of the injection pen 10, a thread 224 is formed for attaching a needle (not shown). The thread 224 surrounds an opening 252 at a proximal end of the cartridge holder 52. The opening 252 is configured to receive a needle that is in fluid connection with an interior of the cartridge 48 inserted into the cartridge holder 52.

The cartridge key 52 forms an inner surface 254 (FIG. 22B) that defines a cylindrical receptacle that receives the cartridge 48 and prevents tilting of the cartridge 48 with respect to the longitudinal axis. Tilting of the cartridge 48 is prevented by an areal contact between a cylindrical outer surface 256 of the cartridge and the inner surface 254 in a holding section 249 of the cartridge holder 52. Furthermore, the cartridge holder 52 forms a cut-out 221 to receive the bypass 206 of the dual chamber cartridge 48. The bypass 206 form-fittingly engages the cut-out 221 so that the dual chamber cartridge 48 is axially and rotationally fixed to the cartridge key 52. On the opposite side of the cut-out 221, a slot 223 is formed extending in the axial direction. The slot 223 allows to reversibly widen the cartridge key 52 to axially insert the dual chamber cartridge 48 with the bypass 206.

In order to mix the different components in the dual chamber cartridge 48 and to prime the injection pen 10, the cartridge container 50 is screwed onto the piston rod guide 42 until a distal end surface 226 of the cartridge container 50 abuts a proximal surface 228 (cf. FIG. 15B) of the piston guide 42. In order to do so, the cartridge container 50 forms an outer surface 246 that can be grasped with one hand to screw the cartridge container 50 onto the piston rod guide 42. When the cartridge container 50 is screwed onto the piston rod guide 42, a proximal portion of the piston rod guide 42 enters an annular space 258 (FIG. 23B) between an outer circumferential surface 264 of the cartridge holder 52 and an inner circumferential surface 260 of the cartridge container 50 defining an inner space 248 of the cartridge container 260. In order to screw the cartridge container 50 onto the piston rod guide 42, a first thread 230 is formed on the inner circumferential surface 260 of the cartridge container 50 that is engaged with a second thread 232 formed on the outer circumferential surface 262 of the piston rod guide 42. As can be seen in FIG. 15A, the piston rod guide 42 forms a snap element 234. The snap element 234 allows screwing, i.e. a compulsory guided combined axial and rotational movement, of the cartridge container 50 relative to the piston rod guide 42 in the distal direction but blocks screwing of the cartridge container 50 relative to the piston rod guide 42 in the proximal direction 1 if the snap element 234 engages with one of the openings 236, 238, and 240. The first opening 236 (cf. FIG. 20B) is configured to define a starting position of the cartridge container 50 and makes sure that the cartridge container 50 cannot be detached from the piston rod guide 42. This starting position or as-delivered state is shown in FIGS. 25A and 25B.

The second opening 238 defines a reconstitution state of the cartridge container 50. In this state, the second chamber 202 still contains air so that the injection pen 10 can be moved forth and back to ensure that the drug is homogenously mixed together. The second opening 238 can be omitted. Therefore, the present disclosure is also directed at an embodiment of the injection pen 10 that features the first 236 and third opening 240 but not the second opening 238. The third opening 240 defines a knob cover unfastening state of the cartridge container 50 where most of the air is expelled from the second chamber 202, which now contains the reconstituted medicament ready for use.

In the following with regard to FIGS. 23A to 33B, different states of the injection pen 10 are described during usage of the pen 10.

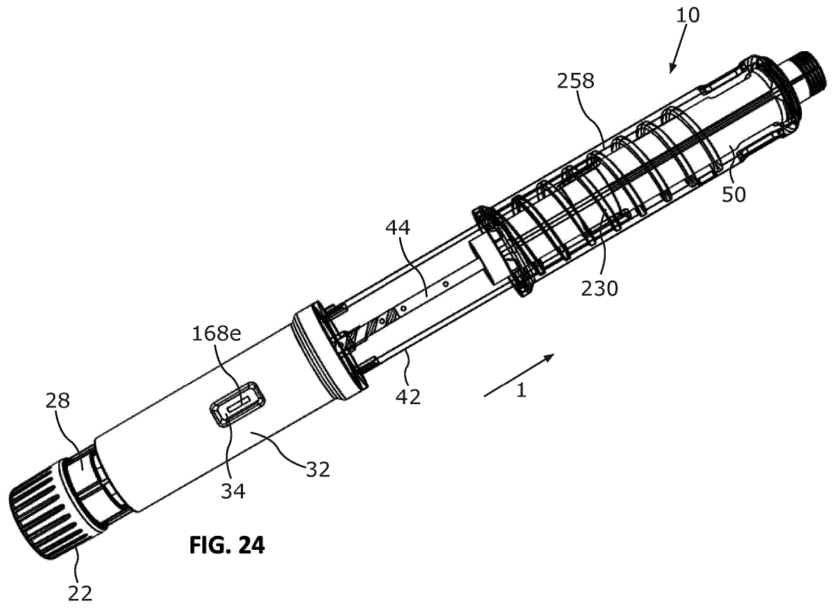

FIGS. 23A to 25B depict the injection pen 10 in the as-delivered state. As can be seen in FIG. 23A, the knob cover 16 covers a distal end section of the injection pen 10 up to a joint between the housing 32 and the piston guide 42. Therefore, the dose setting knob 22 is fully covered by the knob cover 16 so that it is not possible for the user to prematurely set a dose in this state. Looking at FIG. 23B, it can be seen that in the as-delivered state, the drug reconstitution unit 56 forms two separate chambers 202, 204 divided by the first sealing element 208. That means that the two components of the drug, each being stored in one of the two chambers 202, 204 are not yet mixed together. As can be seen in FIG. 24, where the knob cover 16 is blanked out to show what is under the knob cover 16, the dose setting sleeve 34 indicates that the injection pen 10 is in a preset state which differs from a zero-dose state. Accordingly, the dose setting knob 22 is also in a preset position differing from a zero-dose position. As can be seen in FIG. 25A and 25B, the snap element 234 of the piston rod guide 42 is snapped into the first opening 236 of the cartridge container 50. In FIG. 24, the cartridge container 50 is depicted as semi-transparent in order to show the first thread 230 formed on the inner circumferential surface of the cartridge container 50. Secondly, the piston rod guide 42 is also depicted as semi-transparent to show the position of the piston rod 44 in the preset state.

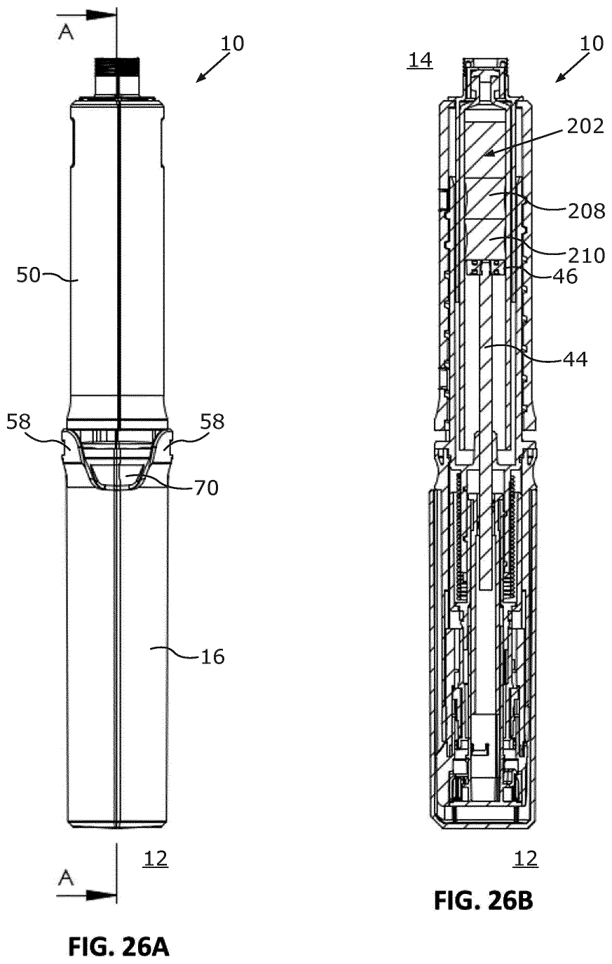

To start preparation of the drug, as can be seen from comparing FIGS. 25A and 26A, the cartridge container 50 is rotated by the user which causes the cartridge container 50 including the cartridge key 52 and the dual chamber cartridge 48 to move in the distal direction relative to the piston rod guide 42. The piston rod guide 42 thereby moves into the annular space 258 (FIG. 23B) between the cartridge container 50 and the cartridge holder 52. The piston disc 46 is snapped to the piston rod 44, which is rotationally fixed by the piston rod guide 42 and axially fixed by the nut 38. The piston disc 46 thus blocks the movement of the second sealing element 210 arranged in the dual chamber cartridge 48 so that the second sealing element 210 slides along the inner circumferential surface of the dual chamber cartridge 48 while the cartridge container 50 is further screwed onto the piston rod guide 42. The solvent stored in the second chamber 204 pushes against the first sealing element 208 which also causes the first sealing element 208 to slide along the inner circumferential surface of the dual chamber cartridge 48. This would cause an overpressure in the cartridge, but the air can escape through the double-ended needle the user attached to thread 224. When the first sealing element 208 reaches the bypass 206 (cf. FIG. 23B), the first chamber 202 and the second chamber 204 are connected by the bypass 206 and therefore, the lyophilized drug stored in the first chamber 202 and the solvent stored in the second chamber 204 mix.

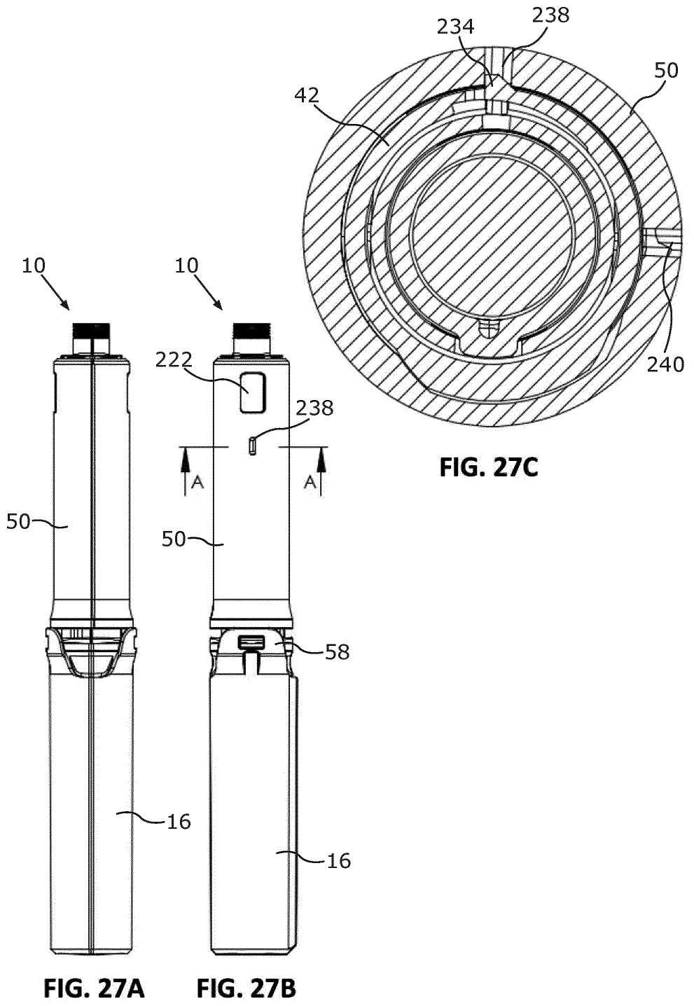

In the reconstitution state shown in FIGS. 26A to 27C, the mixed drug is stored in the first chamber 202 between the first sealing element 208 and the proximal end 14 of the dual chamber cartridge 48. As can be seen in FIG. 26B, a proximal end surface of the second sealing element 210 abuts a distal end surface of the first sealing element 208 so that no second chamber 204 is present anymore in the reconstitution state. As can be seen in FIG. 27C, the snap element 234 of the piston rod guide 42 is snapped into the second opening 238 of the cartridge container 50. In this state, the front chamber 202 still contains a significant amount of air, which helps to create turbulence when moving the pen, so that the mixing of the lyophilized drug is easier. As mentioned before, the second opening 238 can be omitted. In that case the mixing takes place with a low residual amount of air.

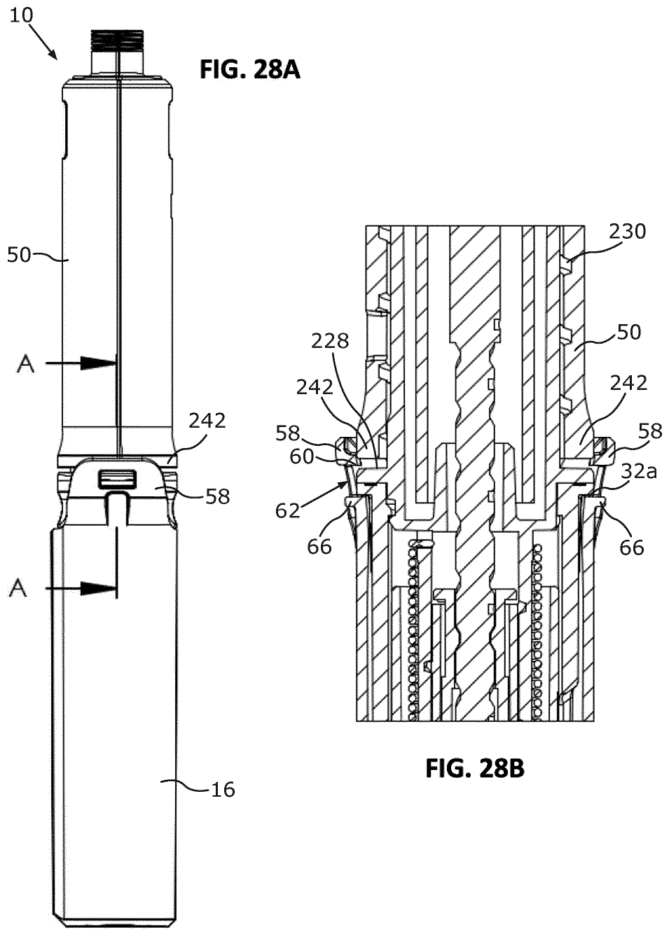

After the reconstitution of the drug is finished, the cartridge container 50 is further rotated by the user causing the cartridge container 50 to move further axially in the distal direction relative to the piston rod guide 42. This causes a displacement section 242 positioned at a distal end of the cartridge container 50 to engage with and spread the wings 58 of the knob cover 16 radially outwardly (cf. FIG. 28B). Spreading the wings 58 radially outwardly causes the form-fitting engagement means 60 of the knob cover 16 to disengage from the coupling surface 228 so that the knob cover 16 is axially movable relative to the housing 32. It is now possible to pull off the knob cover 16 from the housing 32 in the distal direction resulting in the state shown on FIG. 29A and 29B. When the cartridge container 50 is fully screwed onto the piston rod guide 42, a radial end stop 244 formed on the outer circumferential surface 262 of the piston rod guide 42 abuts a radial end stop (not shown) on an inner circumferential surface of the cartridge container 50. Furthermore, the snap element 234 of the piston rod guide 42 is snapped into the third opening 240. Consequently, the cartridge container 50 is rotationally locked to the piston rod guide 42 and the housing 32 of the device. Therefore, movement of the cartridge container 50 and the cartridge 48 respect to the housing 32 and the piston rod guide 42 is inhibited.

As can be seen in FIG. 29A and 29B, at this stage the knob key 30 is still clipped onto the outer circumferential surface of the dose selector 28 between the proximal edge 160 of the dose setting knob 22 and the distal edge 162 of the housing 32. The knob key 30 can be taken away from the dose selector 28 only after the knob cover 16 has been removed by slightly bending the knob key 30.

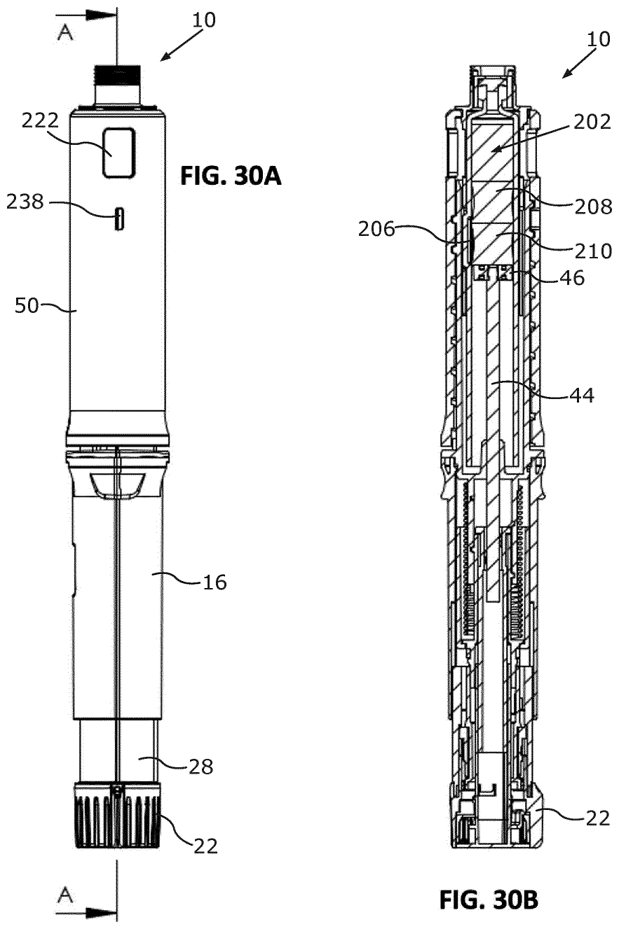

Afterwards, as can be seen when comparing FIGS. 29A and 30A, the dose setting knob 22 is rotated by the user the set a desired dose out of multiple possible settable doses. In this example, the dose setting knob 22 is rotated 180° to set the desired dose. While the dose setting knob 22 is rotated, the dose setting knob 22 makes a compulsory guided combined axial and rotational movement, namely a screw movement, in the distal direction.

Rotating the dose setting knob 22 causes rotation of the injection button 18, that is axially and rotationally connected to the dose setting knob 22 via the snap ring 20, the snap element 24, which is rotationally connected to the dose setting knob 22 via the teeth 108 intermeshing with the teeth 110, the driver 36, which is rotationally and axially coupled to the snap element 24, and the dose setting sleeve 34 which is rotationally and axially coupled to the driver 36. Rotation of the driver 36 causes the driver 36 to move axially in a distal direction due to the engagement of the outer thread 170 of the driver 36 and the inner thread 172 of the piston rod guide 42. The axial movement of the driver 36 causes the snap element 24 to move in a distal direction which pushes the injection button 18 and the dose setting knob 22 in the distal direction via the couplings means 102 of the snap element 24 interacting with the assembling means 98 of the injection button 18. This causes the dose setting knob 22 to perform a compulsory guided combined axial and rotational movement during dose setting.

Furthermore, rotating the dose setting knob 22 causes rotation of the injection button 18 that is rotationally coupled to the nut 38. Since the piston rod 44 is rotationally fixedly coupled to the piston rod guide 42 due to their corresponding out of round cross-sections 186, 188, the nut 38 moves in the distal direction when the dose setting knob 22 and therefore the nut 38 is rotated.

The amount of axial movement of the nut 38 relative to the piston rod 44 and the driver 36 relative to the piston guide 42 depends on the pitch of the respective thread. The outer thread 170 of the driver 36 has a greater pitch than the outer thread 190 of the piston rod 44 so that the driver 36 moves in the distal direction more than the nut 38. For example, the outer thread 170 of the driver 36 can have a pitch of 10.71 mm and the outer thread 190 of the piston rod 44 can have a pitch of 10.21 mm.

When the desired dose is set, the spiral torsion spring 40 applies a torque to the snap element 24 via the driver 36 to bring the dose definition element 116 in abutment with the respective dose stop 118a to 118d, namely with its side surface 122b. Due to the spring 40, the injection pen 10 is configured to rotationally self-align the snap element 24 and the dose selector 28 in different predefined rotational positions defining predefined doses.

If the user then pushes the injection button 18 on the distal end 12 of the injection pen 10, the dose setting knob 22 moves in the proximal direction 1 relative to the snap element 24. This results in the coupling means 100 being bent while passing the circumferential ledge 102 causes a counterforce in the distal direction which has to be overcome by the user to start the injections process. The dose setting knob 22 moving in the proximal direction 1 relative to the snap element 24 also results in the teeth 108 of the dose setting knob 22 disengaging with the teeth 110 of the snap element 24 and instead the teeth 108 of the dose setting knob 22 engaging with the teeth 114 of the connector 26. Since the connector 26 is rotationally coupled to the housing 32 via the dose selector 28, the dose setting knob 22 is rotationally fixed to the housing 32. Therefore, during dose delivery, the dose setting knob 22, the injection button 18, the dose selector 28, and the nut 38 do not rotate relative to the housing 32.

If the user further pushes injection button 18, the injection button 18 and the dose selector 28 move relative to the snap element 24 in the proximal direction 1. Thereby, the dose definition element 116 of the snap element 24 passes through the circumferentially extending rib 156 on the dose selector 28 through the respective cut-out 158a-158d corresponding to the set dose. At the same time, the hard stop 126 of the dose selector 28 moves in the axial direction relative to the hard stop 124 on the snap element 24 which allows the dose selector 28 and the snap element 24 to rotate relative to each other past the pre-set dose position towards the zero-dose position.

When the injection button 18 is pushed during dose delivery, the injection button 18 pushes the driver 36 via the snap element 24 in the proximal direction 1. The spring 40 supports the axial movement of the driver 36 by applying a torque to the driver 36 resulting in an axial movement of the driver 36 in the proximal direction 1 due to the outer thread 170 of the driver 36. The driver pushes the nut 38 in the proximal direction 1 which causes the piston rod 44 to move in the proximal direction 1. The movement of the piston rod 44 and the piston disc 46 in the proximal direction 1 causes the drug to be injected into the patient. Since the injection pen 10 is made to inject relatively large amounts of drug, the pen 10 does not have a so-called gearing. In other words, the parts that are configured to rotate relative to the housing during dose delivery are connected to the housing 32. This means that the distance the piston disc 46 advances is essentially equal to the distance the injection button 18 is pushed in the proximal direction 1 relative to the housing 32.

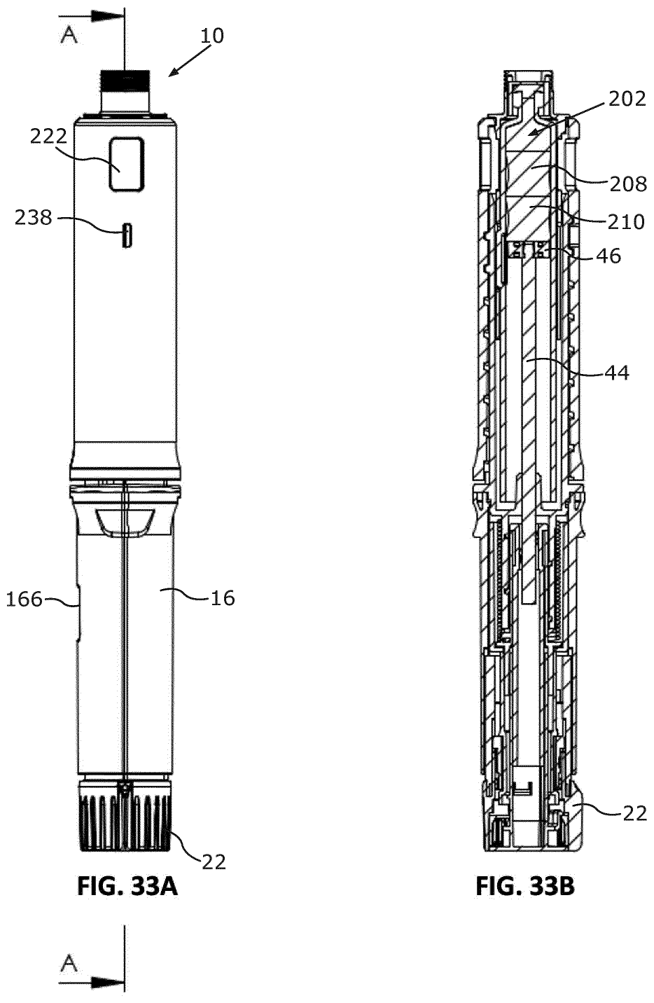

Since the driver rotates relative to the housing due to its outer thread 170, the dose setting sleeve 34 rotates during dose delivery. At the end of the dose delivery (cf. FIG. 33A and 33B) the dose setting sleeve 34 is in a rotational position in which a zero-dose label can be seen through the window 166 of the housing 32. The end of dose stop 174 (cf. FIG. 13B) of the driver 36 and the end of dose stop 176 (cf. FIG. 16C) of the piston rod guide 42 define an end of the movement of the injection button 18 in the proximal direction 1 during dose delivery.

At the end of the dose delivery, the coupling means 100 on the injection button 18 passes the coupling means 102 of the snap element 24 when initiating the injection, which permanently rotationally couples the dose setting knob 22 and the injection button 18 to the housing 32. Thus, the injection pen 10 is rendered inoperable, as the user cannot rotate the dose setting knob 22 to set a new dose.

The injection pen 10 allows for adjusting an axial position of the piston rod 44 with respect to the housing 32 in the preassembled state of the dose delivery mechanism 54. In the preassembled state, the injection button 18, which forms an adjusting element 18 of the dose delivery mechanism 54, engages with its distal assembling means 98a with the coupling means 102 of the snap element 24. This allows to position the injection button 18 in a more distal preassembled position compared to its assembled position in an assembled state of the dose delivery mechanism 54, in which assembled position the adjusting element 18 engages the coupling means 102 with its proximal assembling means 98b.

In the preassembled position, the adjusting element 18 protrudes from the dose setting element 22 and is free to rotate with respect to the dose setting element 22. Rotation of the adjusting element 18 then rotates the nut 38 with respect to the piston rod 44 and thereby causes axial movement of the piston rod 44 due to the threaded connection 189 between the piston rod 44 and the nut 38.

Adjustment of the piston rod 44 in the preassembled state is further detailed below in connection with a second injection pen 330 according to the present disclosure, which is a variant of the injection pen 10 shown in the previous figures.

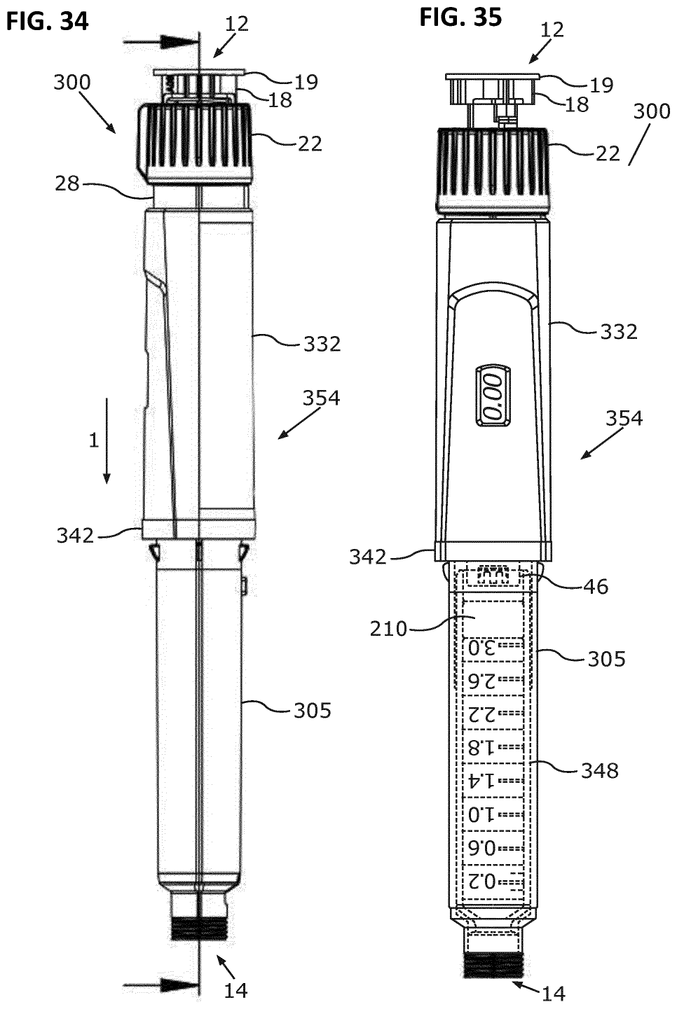

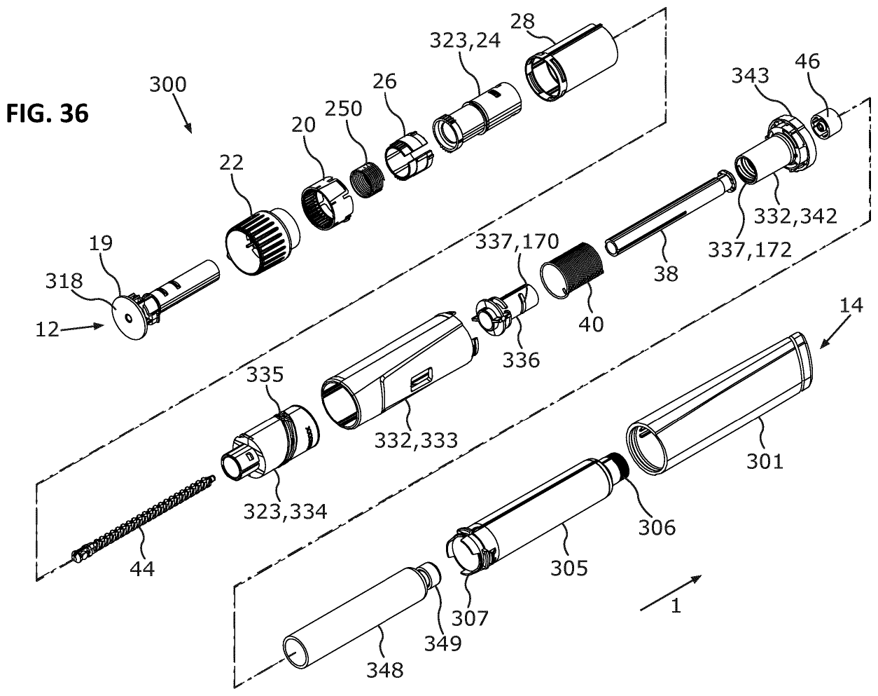

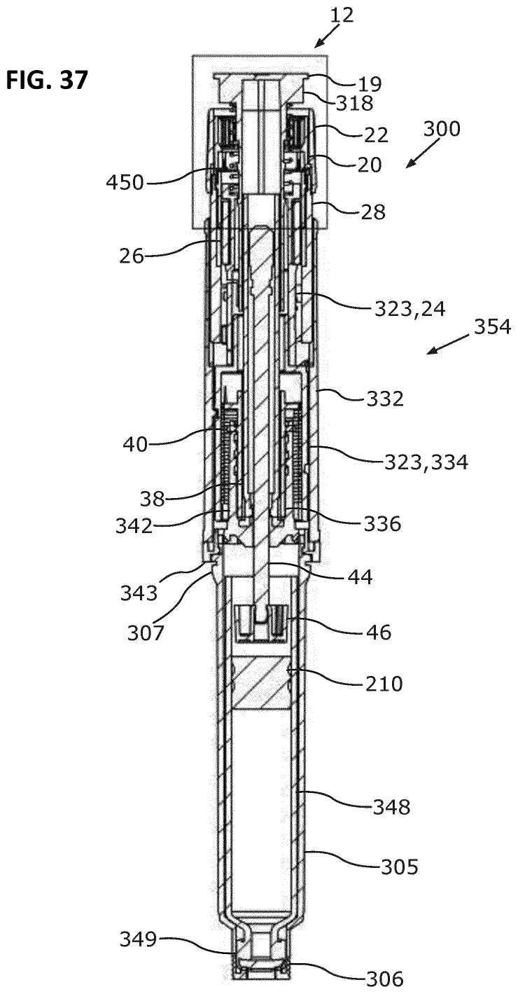

FIGS. 34 and 35 show the second injection pen 300 in the preassembled state, FIG. 36 shows an exploded view of the second injection pen and FIG. 37 shows a longitudinal cut through the second injection pen 300 in the preassembled state. As far as no differences are disclosed in the description or the Figures, the second injection pen 300 is configured as it is disclosed for the injection pen 10 of the previous Figures and vice versa.

The second injection pen 300 comprises a dose delivery mechanism 354. As far as no differences are disclosed in the description or the Figures, the dose delivery mechanism 354 of the second injection pen 300 is configured as it is disclosed for the dose delivery mechanism 54 of the injection pen 10 and vice versa.

The dose delivery mechanism 354 comprises a housing 332 that has an upper housing part 333 and a piston rod guide 342 that forms a lower housing part. The upper housing part 333 and the piston rod guide 342 are rigidly connected to each other via a form-fit connection. In particular, the upper housing part 333 and the piston rod guide 342 are axially and rotationally fixed to each other. The lower housing part formed by the piston rod guide 342 is configured to connect to a medicament container holder 305 that receives a medicament container 348. The medicament container holder 305 comprises a connector 307 that is located at a distal end of the medicament container holder 305. The connector 307 is configured to connect to a corresponding connector 343 of the piston rod guide 342, the corresponding connector 343 being accessible at a proximal side of the piston rod guide 342. The connectors 307, 343 provide a non-releasable form-fit connection between the medicament container holder 305 and the housing 332 after attachment of the medicament container holder 305 to the housing 332.

The medicament container 348 has a single medicament chamber that is sealed by a single plunger 210 at its distal end (see FIG. 35). The medicament chamber contains a fluid medicament. At its proximal needle end 349, the medicament container 348 comprises a septum that is configured to be punched upon attaching a double-sided cannula to a needle connector 306 located at the proximal end of the medicament container holder 305. A cap 301 is releasably attachable to the medicament container holder 305 during storage of the injection pen 300.

The dose delivery mechanism 354 comprises an injection button that constitutes an adjusting member 318, a snap element 24, a dosing element 334 and a driver 336. In the preassembled state and in the assembled state of the injection pen 300, the snap element 24 and the dosing element 334 are rigidly connected to each other and form a dosing member 323 of the dose delivery mechanism 354. The dosing element 334 is coupled to a housing 332 of the dose delivery mechanism 354 via a threaded connection 335. The threaded connection 335 comprises an outer thread on an outer surface of the dosing member 323 and an inner thread (not visible in FIG. 36) on an inner surface of the housing 332. With other embodiments, the dosing member 323 can also be configured as a single component.

The dosing member 323 constitutes a dose indication member of the dose delivery mechanism 354. Thereby, the dosing element 334 comprises markings that are visible through a window in the upper housing part 333 of the housing 332 upon rotation of the dosing member 323 with respect to the housing 332 during dose setting.

The driver 336 is connected to the housing 332 via a further threaded connection 337 that acts between the driver 336 and the piston rod guide 342, as it is described for the driver 36 and the piston rod guide 42 of the injection pen 10. The driver 336 is furthermore rotationally fixed and axially movable with respect to the dosing member 323 via a splined connection. Thereby, the driver 336 is received within the dosing element 334 of the dosing member 323. The splined connection comprises first spline elements on the outer circumference of the driver 336 that engage with corresponding second spline elements on the inner circumference of the dosing element 334. Simultaneous rotation of the driver 336 and the dosing member 323 requires axial movement of the driver 336 due to the further threaded connection 337 to the housing 332 and simultaneous axial movement of the dosing member 323 due to the threaded connection 335 to the housing 332.

A pitch of the threaded connection 335 between the dosing element 334 and the housing 332 deviates from a pitch of the further threaded connection 337 between the driver 336 and the housing 332. A ratio of these pitches defines a mechanical advantage of the dose delivery mechanism 354 during dose delivery and a forced proximal movement of the dosing member 334 by a first axial distance leads to a proximal movement of the driver 336 by a second axial distance that deviates from the first axial distance.

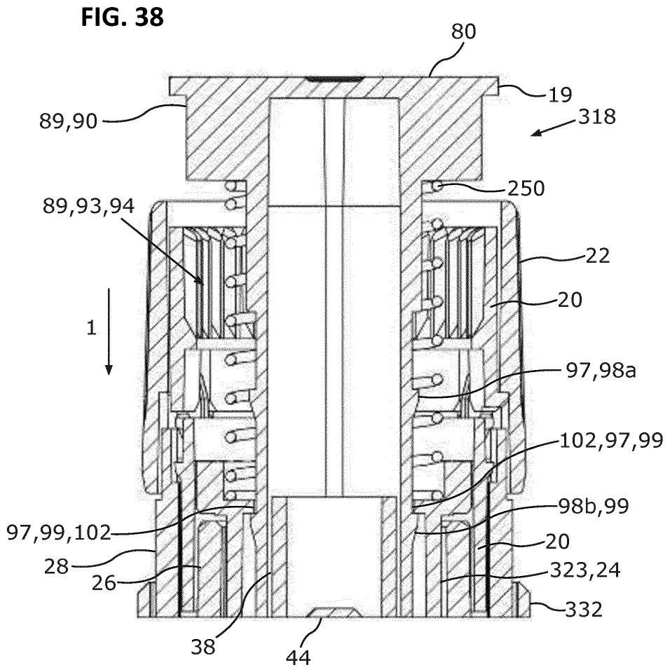

FIG. 38 shows a detailed view of a distal portion of the second injection pen 300 in the preassembled state. The adjusting element 318 deviates from the adjusting element 18 of the injection pen 10 in that it does not feature the coupling means 100 to axially lock the adjusting element 18 to the snap element 24 upon dose delivery. The second injection pen 300 therefore allows to repeatedly set and inject user definable doses. Apart from this modification, the adjusting element 318 is configured as it is disclosed for the adjusting element 18 and vice versa. In particular, the adjusting element 18 of the injection pen 10 is configured to adjust the position of the piston rod 46 in the preassembled state in the same way as it is disclosed in the following for the adjusting element 318 of the second injection pen 300. Furthermore, the adjusting element 318 forms an actuation member of the second injection pen 300. The actuation member is configured to be activated by a user to deliver a set dose. Exemplarily, the actuation member is configured to be pushed in the proximal direction by the user to deliver the set dose.

In the preassembled state shown in FIGS. 37 and 38, the proximal assembling means 98b of the adjusting element 318 engage with the coupling means 102 of the snap element 24 to allow to position the adjusting element 318 in a preassembled position with respect to the housing 332. The preassembled position is the most distal axial position of the adjusting element 318 that is reached when the assembling means 98b engage with the coupling means 102 of the snap element 24 upon distal movement of the adjusting element 318. A biasing element 250 in the form of a spring, which biasing element 250 acts between the snap element 24 and the adjusting element 318, biases the adjusting element 318 in the distal direction into the preassembled position. Since the injection pen 10 does not feature the biasing element 250, the adjusting element 18 of the injection pen 10 is not held in the preassembled position. With the injection pen 10, an assembler of the device manually positions the adjusting element 18 in the preassembled position.

The assembling means 98b form a latch part of a latching mechanism 99 and the coupling means 102 of the snap element 24 form a latch counterpart of the latching mechanism 99. Furthermore, the dosing member 323 with the snap element 24 forms a counter member of the latching mechanism 99. The latching mechanism 99 prevents detachment of the adjusting element 318 from the housing 332 in the preassembled state.

In the preassembled position, the adjusting element 318 distally protrudes from the dose setting element formed by the dose knob 22. The rotation fixation means 90 of the adjusting element 318 then do not engage the rotation fixation means 94 of the snap ring 20 so that the adjusting element 318 is rotationally movable with respect to the housing 332 and the dose setting element 22.

In the preassembled position, an outer rim 19 of the adjusting element 318 is accessible to an assembler of the injection pen 300. When rotating the adjusting element 318 with respect to the housing 332 and the dose setting element 22, the adjusting element 318 rotates the nut 38. The nut 38 thereby does not axially move with respect to the housing 332 since it is restrained by the stationary driver 336 pushing on the pressing surface 194 at the proximal end of the nut 38. The threaded connection 189 between the piston rod 44, which forms a first threaded element, and the rotating nut 38, which forms a second threaded element, then causes the piston rod 44 to axially move with respect to the housing 332. The second injection pen 300 thus allows to adjust the axial position of the piston rod 44 by rotating the adjusting element 318 with respect to the housing 332 and the dose setting element 22. The same holds for the injection pen 10, the injection button 18 of which also forms an adjusting element.

The dose delivery mechanisms 54, 354 of the injection pens 10, 300 each comprise a rotational lock 89, which is formed by the rotational fixation means 90 of the respective adjusting element 18, 318 and the toothed part 93 of the respective snap ring 20. The snap ring 20 thereby forms a connector between the respective adjusting element 18, 318 and the respective dose setting element 22 and the adjusting element 18, 318 is rotationally and/or axially fixed to the dose setting element 22 in the assembled state via the connector 20. Furthermore, the dose setting element 22 forms a counter element to which the adjusting element 18, 318 is attached in the assembled state of the respective dose delivery mechanism 54, 354.

Furthermore, the axial fixation means 82 of the adjusting elements 18, 318 and the rib 84 of the snap ring 20 each form an axial lock 81 that allows axial movement between the adjusting element 18, 318 and the dose setting element 22 in the preassembled state of the dose delivery mechanisms 54, 354 and that prevents axial movement between the adjusting elements 18, 318 and the dose setting element 22 in the assembled state.

The axial fixation means 82 of the adjusting elements 18, 318 and the rib 84 of the snap ring 20 also form a latching mechanism that acts between the adjusting elements 18, 318 and the counter element formed by the dose setting element 22. In the assembled state of the dose delivery mechanisms 54, 354, the latching mechanism blocks the movement of the adjusting elements 18, 318 from a second position with respect to the counter element into a first position with respect to the counter element. The second position thereby is the proximal position in which the adjusting elements 18, 318 are rotationally and axially fixed to the counter element and the first position is the distal position that the adjusting elements 18, 318 take up in the preassembled state and in which the adjusting elements 18, 318 are rotatable with respect to the counter element.

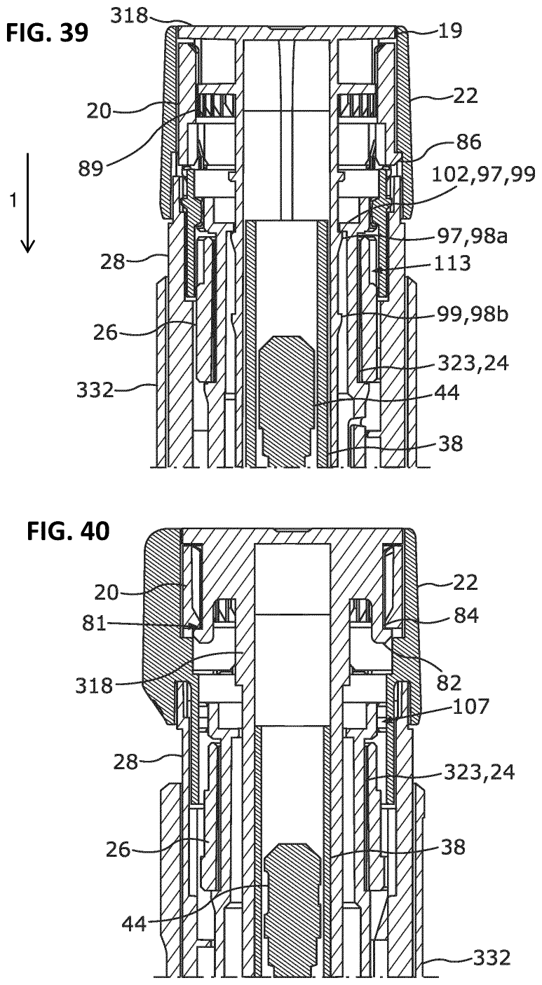

FIG. 39 shows a detailed view of a longitudinal cut through the distal end of the second injection pen 300 in the assembled state during dose setting and FIG. 40 shows a detailed view of a further longitudinal cut through the distal end of the second injection pen 300 in the assembled state during dose setting. Cut planes of the longitudinal cuts shown in FIGS. 39 and 40 are orientated perpendicular to each other.

The injection pen 300 is transferred from the preassembled state into the assembled state by proximally moving the adjusting element 318 from the preassembled position into an assembled position with respect to the dose setting element 22 and the housing 332. This rotationally and axially locks the adjusting element 318 to the dose setting element 22 via the connector 20, the rotational lock 89 and the axial lock 81. In the assembled state, the distal assembly means 98a of the adjusting element 318 engage with the coupling means 102 of the snap element 24 thus irreversibly blocking movement of the adjusting element 318 from the assembled position into the preassembled position.

The distal assembly means 98a of the adjusting element 318 forms a latch part of a latching mechanism 97 that is configured to prevent the adjusting element 318 from moving from the assembled position into the preassembled position with respect to the housing 332. The coupling means 102 of the snap element 24 forms a latch counterpart of the latching mechanism 97 and the dosing member 323 with the snap element 24 forms a counter member of the latching mechanism 97.

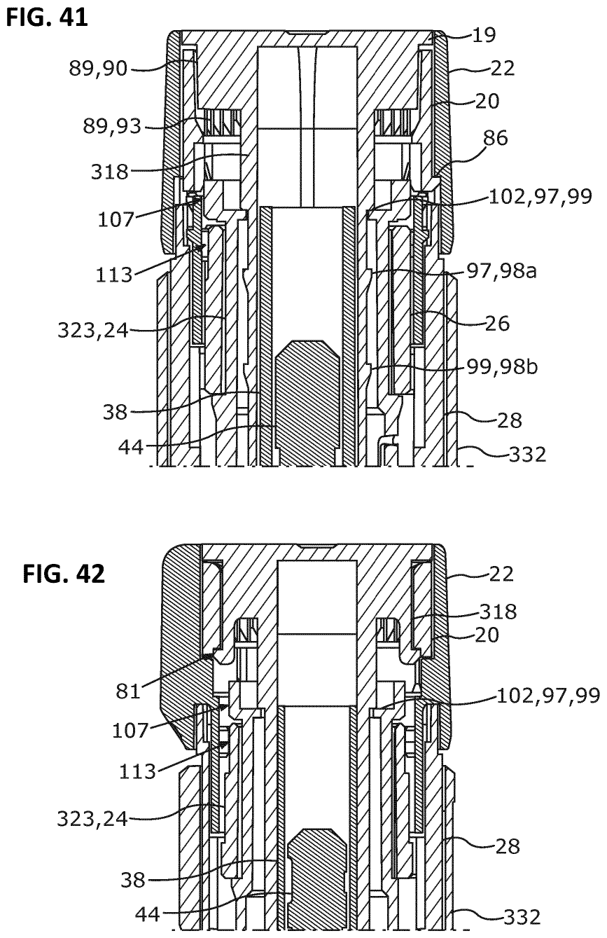

FIG. 41 shows a detailed view of a longitudinal cut through the distal end of the second injection pen 300 in the assembled state during dose delivery and FIG. 42 shows a detailed view of a further longitudinal cut through the distal end of the second injection pen 300 in the assembled state during dose delivery. Cut planes of the longitudinal cuts shown in FIGS. 41 and 42 are orientated perpendicular to each other. FIGS. 41 and 42 thereby show the injection pen 300 at the end of dose delivery when a set dose has been fully expelled and the adjusting element 318 is still pressed by a user of the injection pen 300 in the proximal direction 1.

During dose setting in the assembled state, the adjusting element 318 is rotationally coupled to the dosing member 323 via a clutch mechanism 107 formed by the teeth 108 on the inside surface of the dose setting element 22 (see FIG. 5) and the teeth 110 on the outside surface of the snap element 24 (see FIG. 6). The dose setting element 22 forms a first clutch member of the clutch mechanism 107 and the snap element 24 forms a second clutch member of the clutch mechanism 107. The dosing members 23, 323 of the dose delivery mechanisms 54, 354 each form respective further members of the dose delivery mechanisms 54, 354.

With each dose delivery mechanism 54, 354, the respective clutch mechanism 107 rotationally couples the respective adjusting element 18, 318 to the respective further member in a closed state of the respective clutch mechanism 107 during dose setting in the assembled state and rotationally decouples the respective adjusting element 18, 318 from the respective further member in an opened state of the respective clutch mechanism 107 during dose delivery in the assembled state.

With other embodiments of the clutch mechanisms 107 that couple the adjusting elements 18, 318 to the further members, the second clutch members can also be integrally formed with the further members. For example, when integrally forming the snap element 24 and the dosing element 334 as a single-piece dosing member 323, this dosing member 323 constitutes the further member and, at the same time, the second clutch member.

During dose delivery, the clutch mechanism 107 is opened thus rotationally decoupling the adjusting element 318 and the dosing member 323. In the preassembled state, the clutch mechanism 107 is closed but the adjusting element 318 is rotationally decoupled from the clutch mechanism 107 so that the clutch mechanism 107 does not transfer rotation of the adjusting element 318 to the dosing member 323. In particular, the adjusting element 318 is rotationally decoupled from both the dose setting element 22 and the dosing member 323 in the preassembled state.

During dose delivery in the assembled state, the adjusting element 318 is rotationally coupled to the housing 332 via a further clutch mechanism 113, whereby the further clutch mechanism 113 is formed by the teeth 108 on the inside surface of the dose setting element 22 and the teeth 114 located on the outside surface of the connector 26. The dose setting element 22 thereby forms a first clutch member of the further clutch mechanism 113 and the connector 26 forms a second clutch member of the further clutch mechanism 113. If the further clutch mechanism 113 is in a closed state and the first clutch member engages with the second clutch member, the adjusting element 318 is rotationally fixed to an additional member, the additional member being formed by the housing 332.

During dose setting, the further clutch mechanism 113 is opened so that the adjusting element 318 is allowed to rotate with respect to the housing 332. During dose delivery, the further clutch mechanism 118 disclosed so that the adjusting element is rotationally fixed with respect to the housing. In the preassembled state, the adjusting element 318 is rotationally decoupled from the further clutch mechanism 113, since it is allowed to rotate with respect to both the first clutch member formed by the dose setting element 22 and the second clutch member formed by the connector 26.

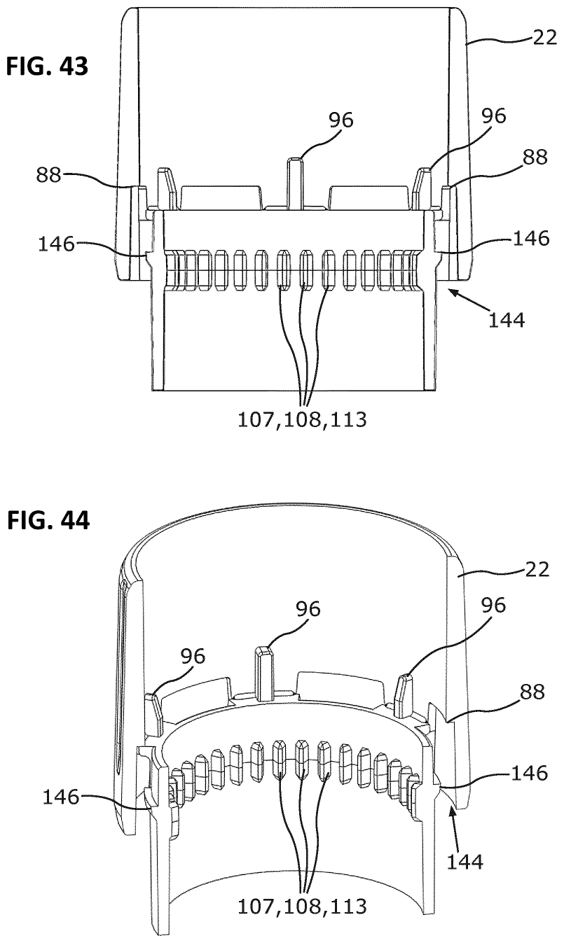

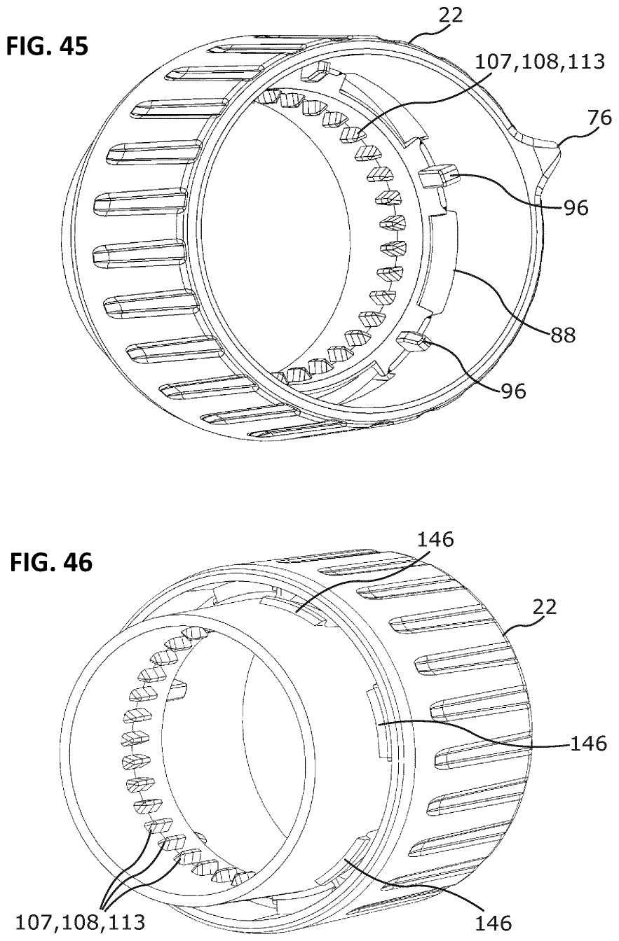

FIG. 43 shows a longitudinal cut through the dose setting element 22 of the injection pen 10 and the second injection pen 300, FIG. 44 shows a perspective view of the longitudinal cut through the dose setting element 22, FIG. 45 shows a perspective distal view of the dose setting element 22 and FIG. 46 shows a prospective proximal view of the dose setting element 22 with the teeth 108 of the clutch mechanisms 107, 113.

With both the clutch mechanism 107 and the further clutch mechanism 113, the clutch mechanisms 107, 113 are in a closed state during one of dose setting and dose delivery and the clutch mechanisms 107, 113 are in an opened state during the other one of dose setting and dose delivery. The clutch mechanism 107 thereby is closed when the further clutch mechanism 113 is opened and the clutch mechanism 107 is opened when the further clutch mechanism 113 is closed.

Furthermore, with each clutch mechanism 107, 113, the adjusting element 318 takes up a dose setting position with respect to the respective second clutch member 24, 26 during dose setting and it takes up a dose delivery position with respect to the respective second clutch member 24, 26 during dose delivery. The dose delivery position thereby is axially shifted with respect to the dose setting position. Exemplarily, the dose delivery position is axially shifted in the proximal direction 1.

With the dose delivery mechanisms 54, 354, the further clutch mechanism 113 also forms a locking mechanism that is configured to rotationally lock the adjusting element 18, 318 to the housing 32, 332 during dose delivery in the assembled state.

The dose selectors 28 of the dose delivery mechanisms 54, 354 each form a retaining member of the respective dose delivery mechanism 54, 354. Each adjusting element 18, 318 is located in a first axial position with respect to the retaining member in the preassembled state and each adjusting element 18, 318 is transferred from the first axial position into a second axial position with respect to the retaining member transferring the respective dose delivery mechanism 54, 354 from the preassembled state into the assembled state. Each adjusting element 18, 318 is rotatable with respect to the retaining member in the preassembled state.

During dose setting in the assembled state, each adjusting element 18, 318 is rotatable with respect to the respective retaining member and, during dose delivery in the assembled state, each adjusting element 18, 318 is rotationally fixed with respect to the respective retaining member. With the dose delivery mechanisms 54, 354, each adjusting element 18, 318 is axially fixed with respect to the respective retaining member in the assembled state.

With the dose delivery mechanisms 54, 354, the first threaded element formed by the piston rod 44 is axially stationary with respect to a third element of the dose delivery mechanism 54, 354 during dose setting in the assembled state. The third element thereby is the housing 32, 332. Furthermore, the second threaded element formed by the nut 38 is axially moved with respect to the third element during dose setting in the assembled state. During adjustment of the piston rod 44 in the preassembled state, the first threaded element formed by the piston rod 44 is axially moved with respect to the third element formed by the housing 32, 332 and the second threaded element formed by the nut 38 is axially stationary with respect to the third element formed by the housing 302, 332.

The dose definition mechanism 115 acting between the snap element 24 and the dose selector 28 of the dose delivery mechanisms 54, 354 is not active in the preassembled state since the adjusting element 318 is rotationally decoupled from the snap element 24 so that the snap element 24 does not rotate upon rotation of the adjusting element 318.

With both dose delivery mechanisms 54, 354, the adjusting element 18, 318 is configured to be rotated in the preassembled state until the bearing 46 touches the distal surface of the plunger 210 after having attached the medicament container 48, 348. A method for adjusting the position of the piston rod 44 in the preassembled state of the dose delivery mechanism 54, 354 can comprise a step of attaching the medicament container 48, 348 to the housing 32, 332 and a step of rotating the adjusting element 18, 318 until the bearing 46 touches the distal surface of the plunger 210. The adjusting element 18, 318 then can further be rotated until the rotation requires a predetermined torque. The dose delivery mechanism 54, 354 can then be transferred from the preassembled state into the assembled state.

The adjusting element 18, 380 can also be rotated until the bearing 46 is located at a distance larger than zero from the distal surface of the plunger 210, thus forming a gap between the distal surface of the plunger 210 and the proximal surface of the bearing 46. The distance can, for example, be measured by measuring the position of the bearing 46 with respect to the plunger 210 through the medicament container 305, which can be made from a transparent material.

Alternatively, the method can also comprise a step of adjusting the position of the piston rod 44 by rotating the adjusting element 18, 318 without the medicament container 48, 348 being attached to the housing 32, 332. The method then can comprise a step of placing the dose delivery mechanism 54, 354 in the preassembled state in an assembly jig and rotating the adjusting element 18, 318 until the proximal surface of the bearing 46 touches a reference surface provided by the assembly jig. The reference surface thereby can be located within the proximal cylindrical portion of the connector 43 of the injection pen 10.

For example, with the injection pen 300, the medicament container 348 can be attached to the housing 332 and the adjusting element 318 then can be rotated until a bearing 46 touches the distal surface of the plunger 210. The adjusting element 318 then can be further rotated until the rotation requires a predetermined torque.

With the dose delivery mechanism 54 of the injection pen 10, the proximal part of the piston rod guide 42 forms a connector 43 that is configured to connect the medicament container 48 axially movable to the housing 32 so that medicament container 48 can perform an axial movement from a receiving position into an operating position after connection to the housing 32. The receiving position thereby is defined by the snap element 234 of the connector 43 engaging with the distal opening 236 of the medicament container holder 50, 52. The operating position is defined by the snap element 234 engaging with the proximal opening 240 of the medicament container holder 50, 52 after having screwed the medicament container holder 50, 52 onto the connector 43.

With the injection pen 10, the dose delivery mechanism 54 can be provided without the medicament container holder 50, 52 being attached to the housing 32 and the position of the piston rod 44 can be adjusted by rotating the adjusting element 18 prior to attaching the medicament container holder 50, 52 to the housing 32. For example, the dose delivery mechanism 54 can be placed in an assembly jig. The adjusting element 18 then can be rotated until the bearing 46 touches a reference surface of the assembly jig and the bearing 46 and the piston rod 44 have reached a predetermined position with respect to the housing 32.

The piston rod 44 thereby is adjusted to a position with respect to the housing 32 that ensures that the bearing 46 gets into contact with the plunger 210 during the movement of the medicament container 48 from the receiving position into the operating position. Furthermore, the position of the piston rod 44 is adjusted to ensure that an amount of the liquid medicament is expelled from the medicament container 48 at the end of the movement into the operating position. For example, the position can be adjusted so that the amount of medicament is expelled only during the last quarter turn of the screwing motion of the medicament container holder 50, 52 onto the proximal part of the piston rod guide 42.

With both the first injection pen 10 and the second injection pen 300, the piston rod 44 can be advanced by a first distance into the proximal direction 1 upon proximal movement of the actuation member 18 by a second distance, wherein the second distance is less than 1.5 times the first distance. With the first injection pen 10, the first distance equals the second distance. Furthermore, the actuation member 18 is traveling the second distance while the piston rod 44 travels the first distance. With the second injection pen 300, the ratio between the second distance and the first distance is given by the ratio of the pitch of the threaded connection 335 between the dosing member 323 and the pitch of the further threaded connection 337 between the driver 336 and the housing 332.

Alternative embodiments of the second injection pen 300 can also comprise a single threaded connection between all members of the second injection pen 300 that rotate during dose delivery and the housing 332. Such alternative embodiments can comprise the dosing member 23 of the first injection pen 10. Like with the first injection pen 10, the dosing member 23 can comprise the driver 36, the dosing element 34 and the snap element 24 shown in FIG. 1.

Additionally or alternatively, the second injection pen 300 can also comprise the drug reconstitution unit 56 of the first injection pen 10. Such a second injection pen 300 then can be configured to receive the double chambered cartridge 48 and to perform reconstitution of a lyophilized drug prior to drug delivery.

Additionally or alternatively, the second injection pen 300 can also be configured to permanently axially lock an actuation member, like the actuation member formed by the adjusting element 318, and/or the dose setting element 22 to the dosing member 323 upon dose delivery. The second injection pen 300 then can comprise the coupling means 100 described in connection with the first injection pen 10.

Generally speaking, the first injection pen 10 and the second injection pen 300 each can comprise a blocking mechanism that is configured to prevent setting and/or delivery of a second dose after having delivered a first dose with the respective injection pen 10, 300. The blocking mechanism can be configured to permanently fix the dose setting element 22 at least rotationally to the housing 32, 332 upon dose delivery, such as upon delivery of a first set dose. Additionally or alternatively, the blocking mechanism can be configured to permanently axially fix the actuation member 18 to a counter member, whereby the actuation member 18 is moved relative to the counter member to initiate delivery of a set dose. With the injection pens 10, 300, the respective counter member is exemplarily formed by the dosing members 23, 323.

The blocking mechanism can comprise a first blocking part that engages a second blocking part to prevent setting and/or delivery of the second dose. With the first and second injection pen 10, 300, the first blocking part is exemplarily formed by the coupling means 100 and the second blocking part is exemplarily formed by the coupling means 102.

The injection pen 10, 300 can comprise a clutch mechanism that rotationally locks the dose setting element 22 to the housing during dose delivery in a closed state of the clutch mechanism. The blocking mechanism can permanently fix the dose setting element 22 at least rotationally to the housing 32, 332 by locking the clutch mechanism in the closed state. The clutch mechanism can, for example, be the clutch mechanism 107.

The clutch mechanism can comprise, for example, a first clutch part, such as the teeth 114 of the connector 26, that engages with a second clutch part, such as the teeth 108 of the dose setting element 22, in the closed state of the clutch mechanism and that disengage from the second clutch part in the opened state of the clutch mechanism. For example, the first and second clutch parts can engage and disengage from each other upon axial relative movement with respect to each other. The clutch mechanism can, for example, be locked in the closed state by axially locking the first clutch part to the second clutch part.

Embodiments of the second injection pen 300 can be configured, like the first injection pen 10, to restrain a user from prematurely activating the second injection pen 300. Embodiments of the second injection pen 300 can be configured, like the first injection pen 10, to axially lock the actuation member 318 with respect to the housing 332 prior to setting and delivering a first dose. Like the first injection pen 10, the second injection pen 300 can comprise the knob cover 16 and/or the knob key 30.

Like the first injection pen 10, also the second injection pen 300 can be, in an as-delivered condition, preset to an injectable dose that is higher than zero. For example, the dosing member 323 can be preset to a position that corresponds to a set dose higher than zero. The second injection pen 300 can be, like the first injection pen 10, configured to prevent reduction of the preset injectable dose to zero.

With both the first and second injection pen 10, 300, the dosing member 23, 323 is stopped from rotating in a rotational direction at a first angular position during dose setting and stopped from rotating in the rotational direction at a second angular position during dose delivery, whereby the second angular position is spaced in the rotational direction from the first angular position by a distance that corresponds to the preset dose higher than zero. The second angular position can, for example, be taken by the dosing member 23, 323 at the end of dose delivery.

The first and second injection pen 10, 300 can comprise a first stop that limits rotation in the rotational direction at the first angular position during dose setting and/or a second stop that limits rotation of the rotational direction at the second angular position during dose delivery, for example at the end of dose delivery. The first stop can, for example, be the stop 126 shown in FIG. 9 and/or the second stop can, for example, be the end stops 174, 176 shown in FIGS. 13B and 15D.

With alternative embodiments, a stop that prevents the rotation of the dosing members 23, 323 at the end of dose delivery can also be provided at the respective dose selector 28. The stop can, for example, interact with a corresponding stop provided at the dosing member 23, 323, such as the hard stop 124. Such a stop is disclosed in document WO 2020015980 A1, the disclosure of which is incorporated into the present disclosure in its entirety by reference, including the configuration of the stop, which is referred to as zero dose hard stop in document WO 2020015980 A1.

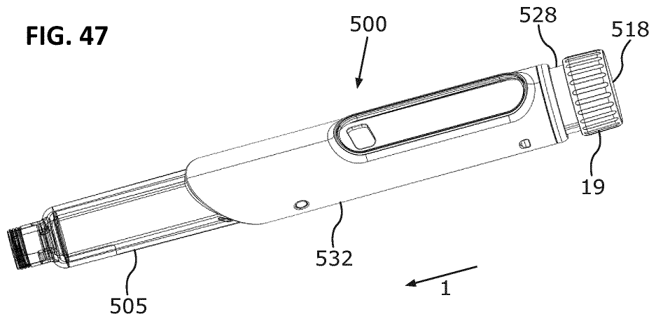

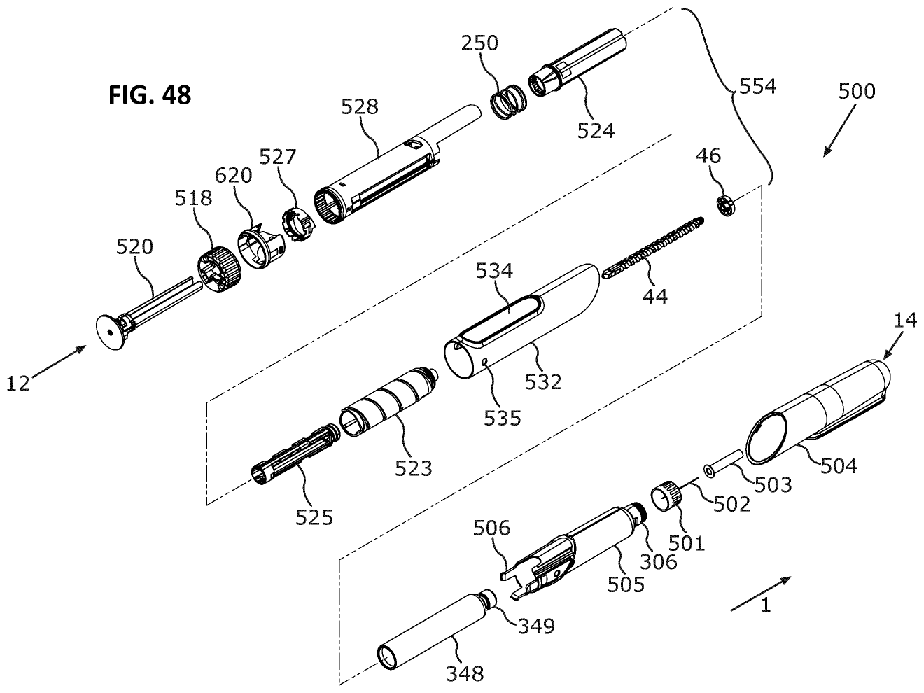

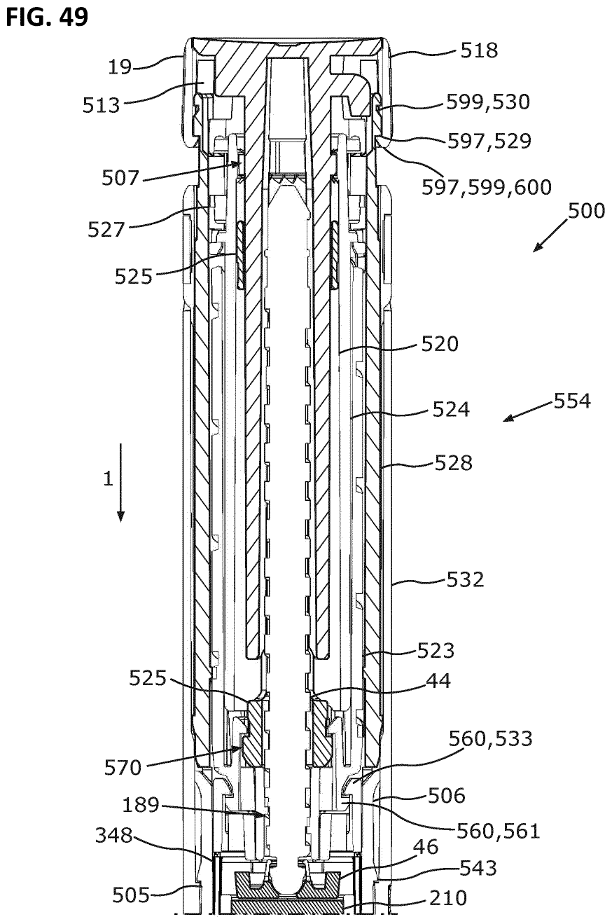

FIG. 47 shows a perspective view of a third injection pen 500 according to the present disclosure, FIG. 48 shows an exploded view of the third injection pen 500 and FIG. 49 shows a longitudinal cut through a dose delivery mechanism 554 of the third injection pen 500 in an assembled state during dose setting. As far as no differences are disclosed in the description or the Figures, the third injection pen 500 is configured as it is disclosed for the second injection pen 300 and vice versa.

The dose delivery mechanism 554 comprises a housing 532 that is configured to connect to a medicament container holder 505 via a non-releasable form-fit connection. The connection comprises a connector 506 located at the distal end of the medicament container holder 505. The connector 506 is configured to engage with a corresponding connector 543 located at the proximal end of the housing 532, see FIG. 49. The connection is configured as a non-releasable snap fit connection.

The medicament container holder 505 is configured to receive the medicament container 348 already described in connection with the second injection pen 300. At a proximal end, the medicament container holder 505 comprises a needle connector 306 that is configured to receive a double ended needle assembly 501 having a double ended cannula 502. The needle connector 300 connects to the needle assembly 501 via a threaded connection. Alternatively, the connection could also be configured as a Luer lock, a snap fit connection or the like. Upon mounting the needle assembly 501 onto the medicament container holder 505, a distal end of the cannula 502 pierces the septum at the proximal needle end 349 of the medicament container 348. The proximal end of the cannula 502 is covered by a needle cap 503 that is removed before use of the injection pen 500. During storage of the injection pen 500, a cap 504 covers the medicament container holder 505.

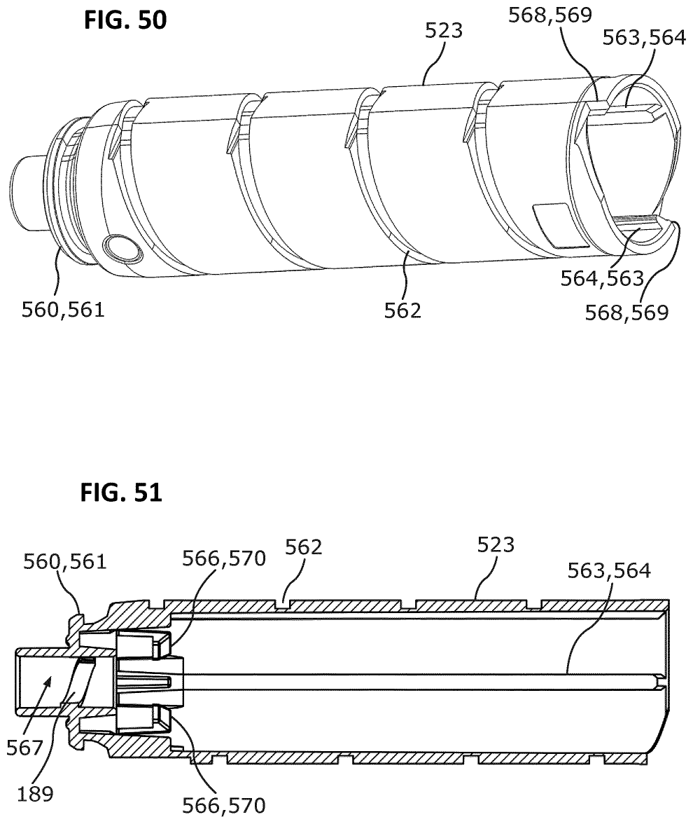

The dose delivery mechanism 554 comprises a dosing member 523 that is axially fixed and rotationally movable with respect to the housing 532 by a rotatable fixation 560. FIG. 50 depicts a perspective distal view of the dosing member 523 and FIG. 51 depicts a longitudinal cut through the dosing member 523. The rotatable fixation 560 comprises an annular rim 561 located at the proximal end of the dosing member 523 and corresponding holding lugs 533 at the proximal end of the housing 532. The holding lugs 533 snap behind the annular rim 561 and thus axially fix the dosing member 523 to the housing 532.

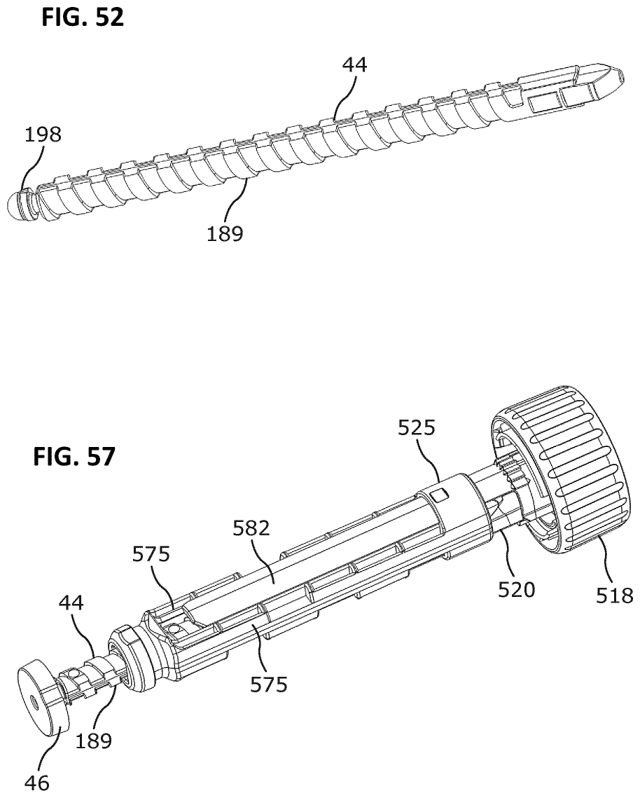

The dose delivery mechanism 554 further comprises a piston rod 44, which is shown in FIG. 52 in a perspective view. The piston rod 44 is received in an opening 567 at the proximal end of the dosing member 523. Thereby, the piston rod 44 is connected to the dosing member 523 via a threaded connection 189. The threaded connection 189 comprises an inner thread within the opening 567 of the dosing member 523 and an outer thread provided at the outer circumference of the piston rod 44. The piston rod 44 forms a first threaded element of the threaded connection 189 and the dosing member 523 forms a second threaded element of the threaded connection 189. At its proximal end, the piston rod 44 comprises coupling means 198 that connect a bearing 46 axially fixed and rotationally movable to the piston rod 44.

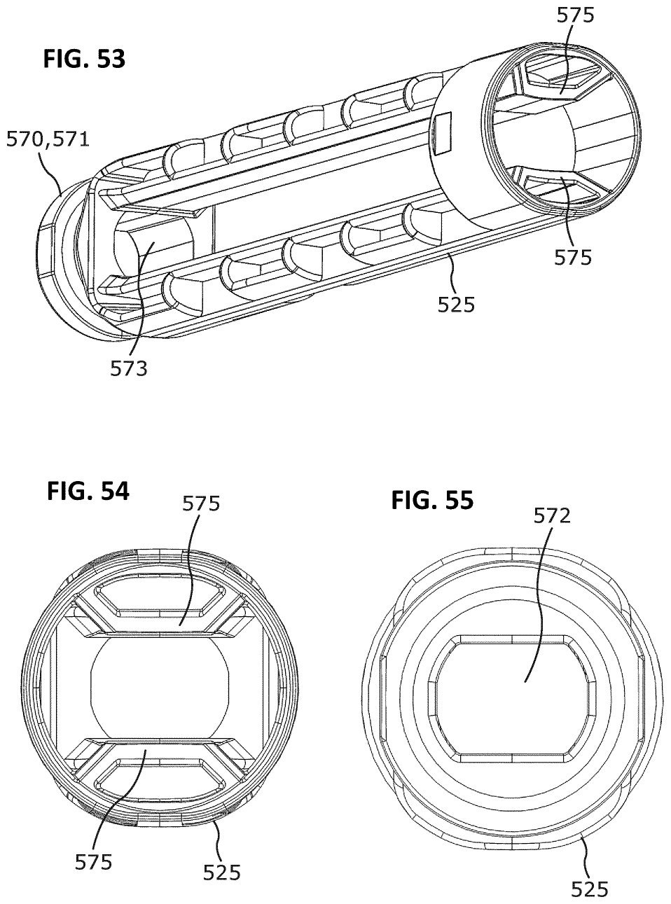

FIG. 53 depicts a perspective distal view of an extension 525 of the dose delivery mechanism 554, FIG. 54 depicts a distal view of the extension 525 and FIG. 55 depicts a proximal view of the extension 525. The extension 525 is received within the dosing member 523. It is held axially fixed and rotationally movable within the dosing member 523 by a rotatable fixation 570. The rotatable fixation 570 exemplarily comprises an annular ridge 571 located at the proximal end of the extension 525 and corresponding lugs 566 provided at the proximal end of the dosing member 523. The lugs 566 snap behind the annular ridge 571 from the proximal side of the extension 525 and thereby axially fix the extension 525 to the dosing member 523.

The piston rod 44 is axially movable and rotationally fixed with respect to the extension 525. At its proximal end, the extension comprises a non-circular opening 573 that is adapted to a corresponding non-circular outer shape of the piston rod 44. The piston rod 44 is received within the opening 573, thereby rotationally locking the piston rod 44 to the extension 525, while allowing relative axial movement between the piston rod 44 and the extension 525.

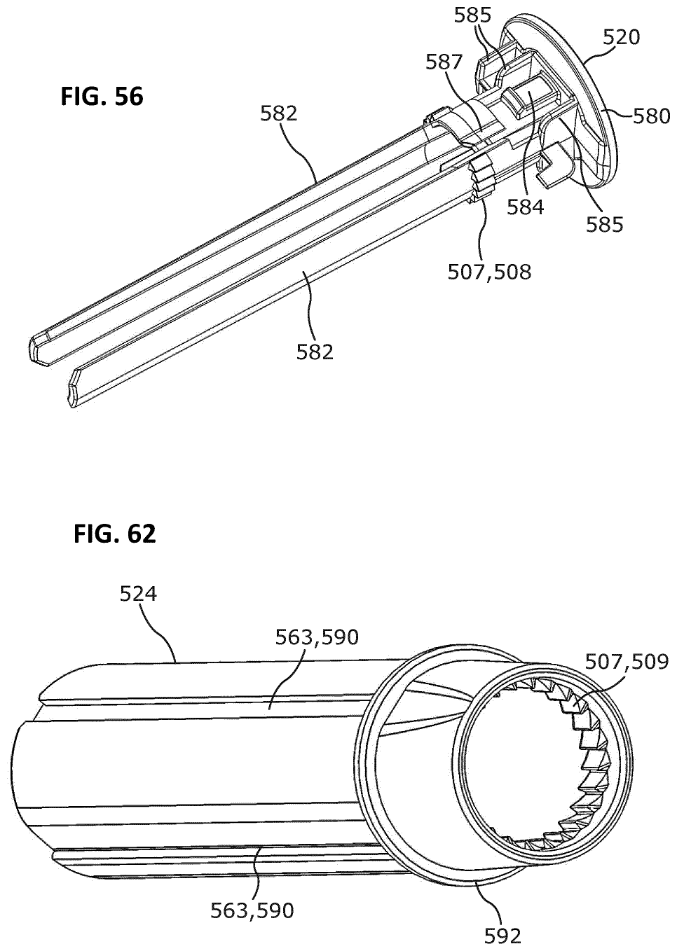

FIG. 56 depicts a perspective view of a coupling element 520 of the dose delivery mechanism 554. The coupling element 520 comprises an end plate 580 located at its distal end and two bars 582 that extend axially and parallel to each other in the proximal direction.

The coupling element 520 is rotationally fixed and axially movable with respect to the piston rod 44. It thereby is coupled to the piston rod 44 via the extension 525. As can be seen from FIG. 57, the bars 582 of the coupling element 520 are received in between two ridges 575 of the extension 525 that radially extend from an inside surface of the extension 525. The ridges 575 thereby run parallel to each other along the axial direction. The bars 582 and the ridges 575 provide an axially movable connection between the coupling element 520 and the extension 525 that rotationally fixes the coupling element 520 to the extension 525.

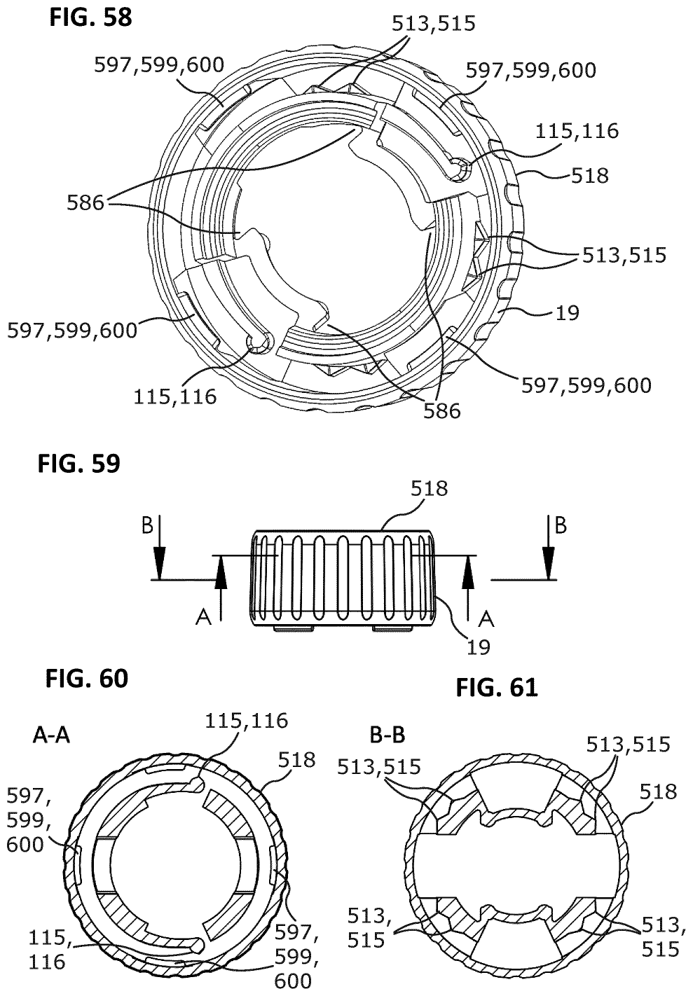

As further can be seen from FIG. 57, an adjusting element 518 is axially and rotationally fixed to the distal end of the coupling element 520. FIG. 58 depicts a proximal perspective view of the adjusting element 518, FIG. 59 depicts a side view of the adjusting element 518, FIG. 60 depicts a radial cut through the adjusting element 518 along the line A-A in FIG. 59 and FIG. 61 depicts a radial cut through the adjusting element 518 along the line B-B in FIG. 59.

The adjusting element 518 is axially and rotationally fixed to the coupling element 520. The adjusting element 518 and the coupling element 520 thus form a single member of the dose delivery mechanism 554. As can be seen from FIG. 56, the coupling element 520 comprises an axial fixation element 584 that is part of an axial fixation acting between the adjusting element 518 and the coupling element 520 and that engages with a corresponding axial fixation element provided at adjusting element 518. The axial fixation element 584 of the coupling element 520 thereby is configured as a snap hook and the corresponding axial fixation element of the adjusting element 518 is configured as a circumferential edge that engages with the snap hook. The coupling element 520 further comprises at least one rotational fixation element 585, for example several rotational fixation elements 585. The rotational fixation elements 585 engage with corresponding rotational fixation elements provided at the adjusting element 518 and thereby rotationally lock the adjusting element 518 to the coupling element 520. The rotational fixation elements 585 of the coupling element 520 are configured is longitudinally ridges that run parallel to the axial direction. The ridges are received in between corresponding longitudinal recesses 586 provided within the adjusting element 518. With other embodiments of the dose delivery mechanism 554, the adjusting element 518 and the coupling element 520 can also be configured as a one-pieced single member.

FIG. 62 depicts a perspective view of a coupling member 524 of the dose delivery mechanism 554. The coupling member 524 is configured as a hollow member. As can be seen from FIG. 49, the coupling member 524 is located in between the dosing member 523 and the extension 525. Thereby, the coupling member 524 is placed within the dosing member 523 and receives the extension 525 in an inner cavity. The coupling member 524 is rotationally fixed to the dosing member 523 via an axially movable rotation fixation 563. The rotation fixation 563 comprises longitudinal recesses 590 provided on an outer surface of the coupling member 524 that engage corresponding longitudinal ridges 564 provided on an inside surface of the dosing member 523, see FIGS. 50 and 51.

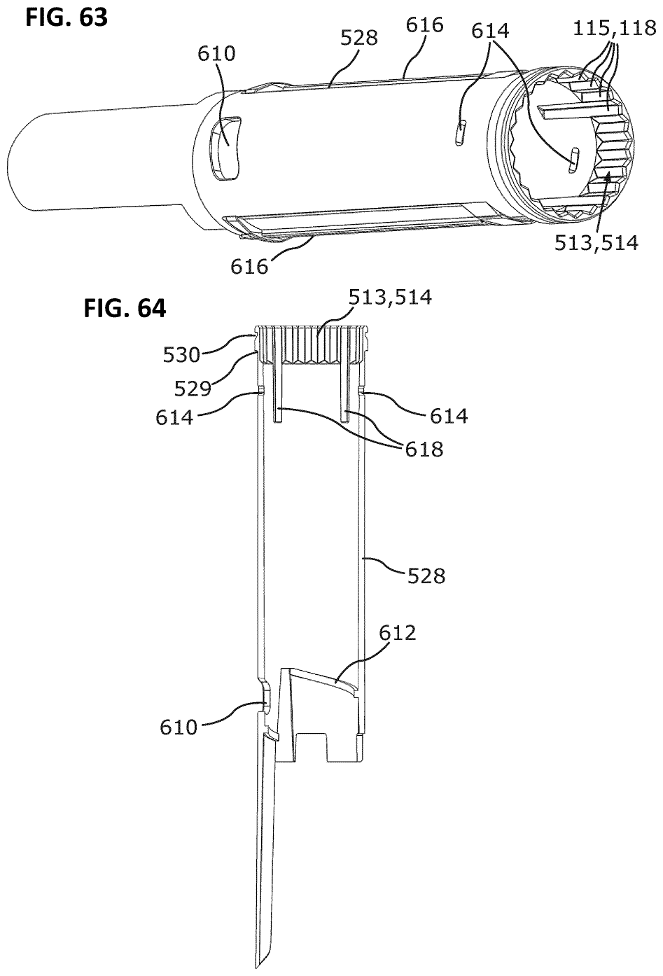

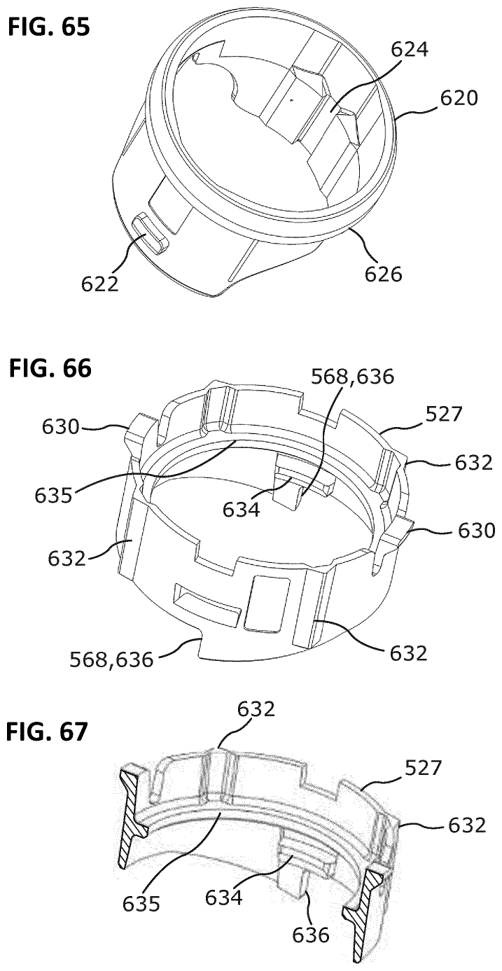

FIG. 63 depicts a perspective distal view of a sleeve 528 of the dose delivery mechanism 554 and FIG. 64 depicts a longitudinal cut through the sleeve 528. The sleeve 528 is configured as a hollow member. As can be seen from FIG. 49, the sleeve 528 is located in between the housing 532 and the dosing member 523.

The sleeve 528 is threadedly connected to and threadedly engaged with the dosing member 523. A threaded connection between the sleeve 528 and the dosing member 523 comprises an inner thread 612 provided on an inside surface of the sleeve 528 that engages an outer thread 562 provided on an outer surface of the dosing member 523.

Furthermore, the sleeve 528 is rotationally fixed and axially movable connected to the housing 532. A connection between the sleeve 528 and the housing 532 thereby comprises a connector 620. The connector 620 is located at the distal end of the housing 532. It is axially and rotationally fixed with respect to the housing 532. With other embodiments, the connector 620 can also be formed integrally with the housing 532. The connector 620 comprises a pair of radial lugs 622 that are provided at an outer surface of the connector 620. The radial lugs 622 engage with corresponding openings 535 accessible at an inside surface of the housing 532. The connector 620 further comprises an outer annular rim 626 provided at a distal end of the connector 620. The annular rim 626 rests against the distal surface of the housing 532, thereby preventing the connector 620 from moving in the proximal direction.

On an inside surface of the connector 620, longitudinal recesses 624 are provided that engages with corresponding longitudinal ridges 616 on an outer surface of the sleeve 528. This provides a rotationally fixed and axially movable connection between the housing 532 and the sleeve 528.

The dose sleeve 523 is configured as a dose indication member and comprises markings on its outer surface that serve to indicate a set dose. A window 610 is formed within the sleeve 528, through which the dose sleeve 523 is visible. The window 610 of the sleeve 528 is aligned with a housing window 534 provided within the housing 532, so that the dose sleeve 523 is visible from the outside of the housing 532. A set dose is then indicated by the marking that is visible through the windows 534, 610.

The coupling member 524 is axially fixed and rotationally movable with respect to the sleeve 528. A connection between the coupling member 524 and the sleeve 528 comprises a connector 527.

FIG. 66 depicts a perspective view of the connector 527 and FIG. 67 depicts a perspective view of a longitudinal cut through the connector 527. The connector 527 is axially and rotationally fixed with respect to the sleeve 528. It comprises longitudinal ridges 632 on its outer surface that engage with corresponding recesses 618 (see FIG. 64) provided on the inside surface of the sleeve 528. Furthermore, the connector 527 comprises radially extending lugs 630 that engage with openings 614 accessible on the inside surface of the sleeve 528. This engagement prevents the connector 527 from being removed from the sleeve 528.

On an inside surface of the connector 527, a distal blocking element 635 and proximal blocking elements 654 are formed. The blocking elements 635, 654 provide an axially fixed and rotationally movable connection to the coupling member 524. Thereby, an annular rim 592 that is provided on the outer surface of the coupling member 524 and that extends in the radial direction (see FIG. 62), is received in between the blocking elements 635, 654.

Furthermore, a radial stop 568 is formed between the sleeve 528 and the dosing member 523. This radial stop 568 is configured to stop relative rotation between the dosing member 523 and the sleeve 528 and thus also between the dosing member 523 and the housing 532 at the end of dose delivery. The radial stop 568 comprises at least one stop surface 569 provided at the dosing member 523 and a corresponding stop surface 636 provided at the connector 527. The stop surfaces 569, 636 are orientated parallel to each other and configured to engage with each other at the end of dose delivery. The stop surfaces 569, 636 form an angle with a radial plane orientated perpendicular to the longitudinal axis of the dose delivery mechanism 554. With the third injection pen 500, the stop surfaces 569, 636 are orientated parallel to the longitudinal axis. While the stop surface 636 is provided at the connector 527, the stop surface 636 can also be provided directly at the sleeve 528 with other embodiments.

With the third injection pen 500, the adjusting element 518 forms a dose setting member of the dose delivery mechanism 554. To set a dose to be delivered, a user rotates the adjusting element 518 with respect to the housing 532 in the assembled state.

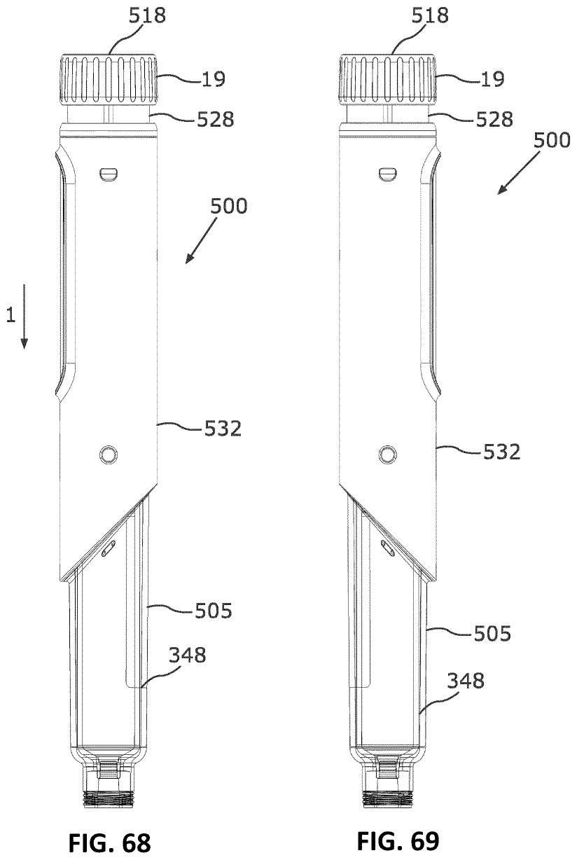

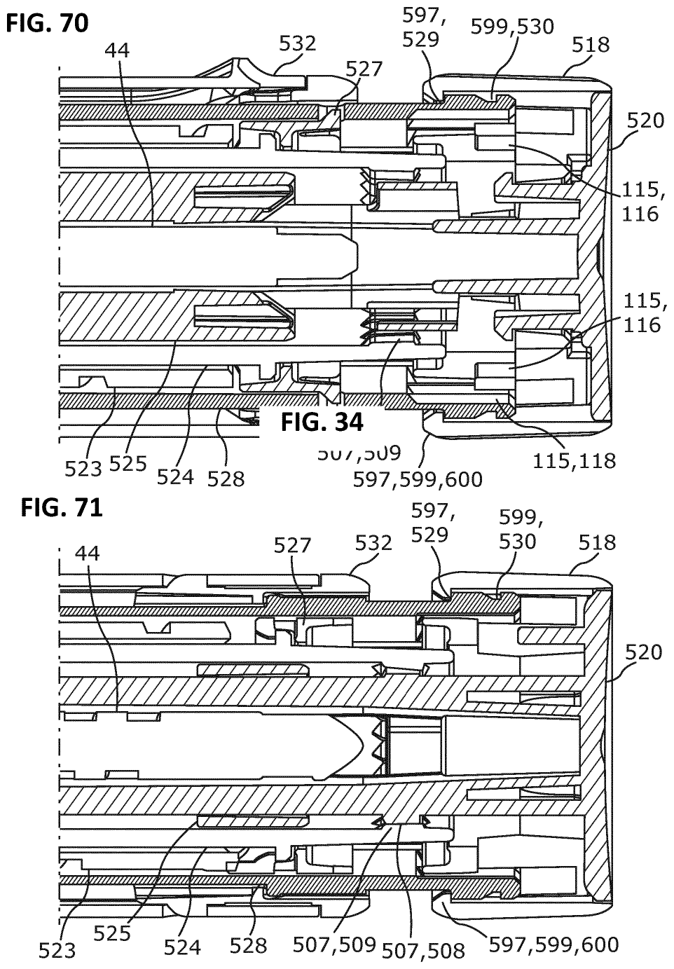

FIG. 68 depicts a side view of the third injection pen 500 in an assembled state during dose setting, when no dose is set. FIG. 69 depicts a side view of the third injection pen 500 in a preassembled state. In the preassembled state, the adjusting element 518 is located in a preassembled position with respect to the housing 532 and in the assembled state during dose setting and with no dose being set, the adjusting element 518 is in an assembled position with respect to the housing 532. Thereby, the assembled position is located more proximally than the preassembled position.

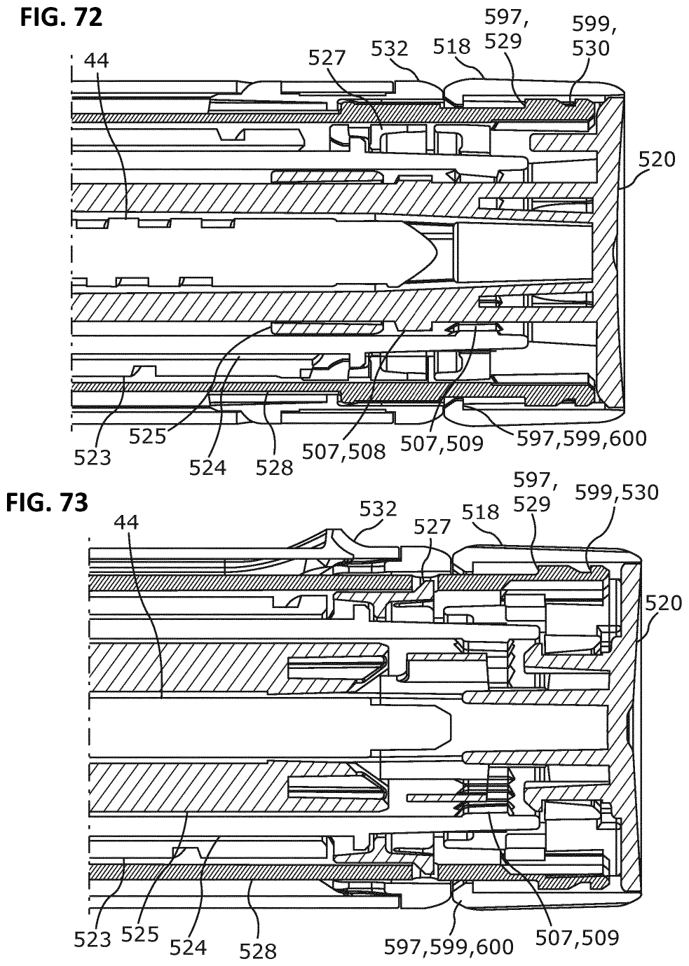

FIG. 70 shows a detailed view of a longitudinal cut through the distal end of the third injection pen 500 in the assembled state during dose setting and FIG. 71 shows a detailed view of a further longitudinal cut through the distal end of the third injection pen 500 in the assembled state during dose setting. Thereby, a longitudinal cut plane of the view shown in FIG. 71 is orientated perpendicular to a longitudinal cut plane of the view shown in FIG. 70.

During dose setting, the adjusting element 518 is rotationally fixed with respect to the coupling member 524 by a clutch mechanism 507. Rotation of the adjusting element 518 then causes rotation of the piston rod 44 due to the rotational fixation via the extension 525 and the coupling element 520 and simultaneous rotation of the dosing member 523 due to the rotational fixation via the coupling element 520, the clutch mechanism 507 and the coupling member 524. Since both the piston rod 44 and the dosing member 523 rotate with respect to the housing 532 at the same speed during dose setting, the piston rod 44 does not change its axial position with respect to the housing 532 despite the threaded connection 189 between the piston rod 44 and the dosing member 523.

Rotation of the dosing member 523 with respect to the sleeve 528 during dose setting causes the sleeve 528 to move axially in the distal direction with respect to the housing 532 due to the threaded connection 562, 612. This also causes distal movement of the adjusting element 518 and the coupling element 520. Furthermore, the coupling member 524 is also moved distally due to the axially fixed and rotationally movable connection to the sleeve 528 via the connector 527.

As can be seen from FIGS. 70 and 71, the adjusting element 518 is coupled to the sleeve 528 by a latching mechanism 597 that prevents distal movement of the adjusting element 518 and the coupling element 520 with respect to the sleeve 528. As can be seen from FIGS. 58 to 61, 63 and 64, the latching mechanism 597 comprises a latch part 600 located at the proximal end of the adjusting element 518 that engages with a latch counterpart 529 of the sleeve 528. The latch counterpart 529 of the sleeve 528 is configured as an annular edge located at the outside surface of the sleeve 528. The latch part 600 of the adjusting element 518 is configured as corresponding radial lugs provided on an inner surface of the adjusting element 518. In the assembled position of the adjusting element 518, the radial lugs 600 engage with the annular edge 529, thus preventing further distal movement of the adjusting element 518 into the preassembled.

The adjusting element 518 and the coupling element 520 are biased with respect to the sleeve 528 in the distal direction by a biasing member 250, which is configured as a compression spring and which is shown in FIG. 48 and which is not visible in FIGS. 70 and 71.

During the rotation of the dosing member 523 and the axial movement of the sleeve 528 with respect to the housing 532 during dose setting, the window 610 of the sleeve 528 axially moves along the dosing member 523. Thereby, a respective marking on the dose sleeve 523 that is visible through the window 610 indicates a dose that is currently set.

To deliver a set dose, a user of the third injection pen 500 pushes the adjusting element 518 and the coupling element 520 in the proximal direction 1 against the force of the biasing member 250.

FIG. 72 shows a detailed view of a longitudinal cut through the distal end of the third injection pen 500 in the assembled state during dose delivery and FIG. 73 shows a detailed view of a further longitudinal cut through the distal end of the third injection pen 500 in the assembled state during dose delivery, whereby a longitudinal cut plane is orientated perpendicular to a longitudinal cut plane of the view in FIG. 73. FIGS. 72, 73 thereby show the dose delivery mechanism 554 of the third injection pen 500 at the end of dose delivery, when the set dose has been fully expelled and the user still presses the adjusting element 518 and the coupling element 520 in the proximal direction.

During dose delivery, the adjusting element 518 and the coupling element 520 are rotationally locked to the housing 532 via the sleeve 528. This is because proximal movement of the coupling element 520 and the adjusting element 518 with respect to the sleeve 528 at the beginning of dose delivery closes a clutch mechanism 513 between the adjusting element 518 and the sleeve 528. The clutch mechanism 513 comprises teeth 515 formed at the adjusting element 518 and corresponding teeth 514 formed at the distal end of the sleeve 528. The clutch mechanism 513 also rotationally locks the piston rod 44 to the housing 532 during dose delivery via the extension 525, the coupling element 520, the adjusting element 518 and the sleeve 528.

Proximal movement of the coupling element 520 with respect to the coupling member 524 at the beginning of dose delivery causes the clutch mechanism 507 between the coupling element 520 and the coupling member 524 to open so that the coupling member 524 becomes rotatable with respect to the coupling element 520. After disengagement of the clutch mechanism 507, further proximal movement of the coupling element 520 pushes the sleeve 528 in the proximal direction 1. The proximal movement of the sleeve 528 rotates the dosing member 523 via the threaded connection 612 between the sleeve 528 and the dosing member 523. Since the piston rod 44 is rotationally locked to the housing 332 during dose delivery, rotation of the dosing member 523 causes proximal movement of the piston rod 44, which proximal movement is driven via the threaded connection 189.

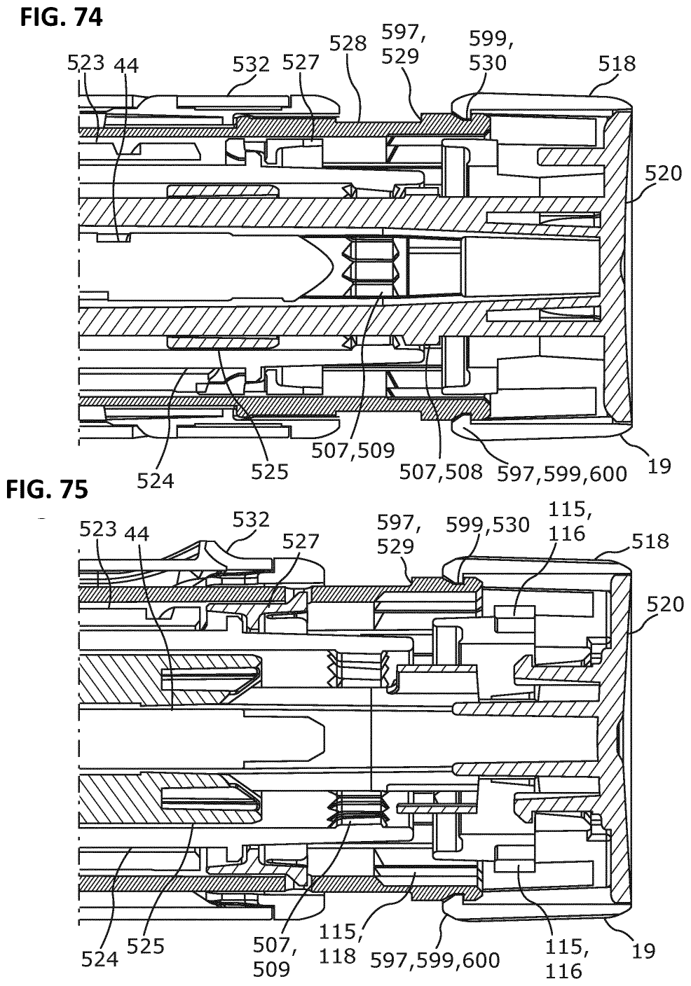

FIG. 74 shows a detailed view of a longitudinal cut through the distal end of the third injection pen 500 in a preassembled state of the dose delivery mechanism 554 and FIG. 75 shows a detailed view of a further longitudinal cut through the distal end of the third injection pen 500 in the preassembled state, whereby a longitudinal cut plane is orientated perpendicular to a longitudinal cut plane of the view in FIG. 74.

In the preassembled state, the adjusting element 518 and the coupling element 520 are located in the adjusting position with respect to the housing 532 and the sleeve 528. In the adjusting position, the adjusting element 518 and the coupling element 520 are shifted in the distal direction with respect to their respective assembled positions in the assembled state.

The dose delivery mechanism 554 comprises a further latching mechanism 599 that prevents detachment of the adjusting element 518 and the coupling element 520 in the preassembled state. The further latching mechanism 599 comprises the latch part 600 of the adjusting element 518 and a further latch counterpart 530 located at the distal end of the sleeve 528. The further latch counterpart 530 thereby is located distally from the latch counterpart 529.

The further latch counterpart 530 is configured as an angular recess that receives the radial lugs of the latch part 600 of the adjusting element 518. The latch parts 600 formed by the radial lugs thereby are releasably engaged with the further latch counterpart 530 and allow proximal movement of the adjusting element 518 while blocking distal movement.

In the preassembled state, the clutch mechanism 507 between the coupling element 520 and the coupling member 524 is opened so that the adjusting element 518 is rotationally decoupled from the dosing member 523. At the same time, the adjusting element 518 is rotationally coupled and rotationally fixed with respect to the piston rod 44 by the coupling element 520 and the extension 525. Rotation of the adjusting element 518 with respect to the housing 532 thereby causes the piston rod 44 to rotate with respect to the housing 532 and the dosing member 523. Due to the threaded connection 189 between the dosing member 523 and the piston rod 44, the piston rod 44 moves axially with respect to the housing 332 upon rotation of the adjusting element 518.

With the clutch mechanism 507, the coupling element 520 forms a first clutch member of the clutch mechanism 507 and the coupling member 524 forms a second clutch member of the clutch mechanism 507. The dosing member 523 of the dose delivery mechanism 554 forms a further member of the dose delivery mechanisms 54, 354 to which the adjusting element 518 is rotationally coupled during dose setting in the assembled state and from which the adjusting element 518 is rotationally decoupled during dose delivery in the assembled state.

The coupling element 520 comprises a first clutch part 508 of the clutch mechanism 507. The first clutch part 508 is configured as radial teeth that are provided on an outer surface of the coupling element 520. The coupling member 524 comprises a second clutch part 509 of the clutch mechanism 507. The second clutch part 509 is configured as radial teeth that are located on the inside surface of the coupling member 524. In the closed state of the clutch mechanism 507, the first clutch part 508 is engaged with the second clutch part 509, as it is shown in FIG. 71.

During the adjustment of the piston rod in the preassembled state, the clutch mechanism 507 is in an opened state. Thereby, the first clutch part 508 and the second clutch part 509 are brought out of engagement by locating them at an axial distance from each other. The first clutch part 508 thereby is shifted in a distal direction from the second clutch part 509, the distal direction being opposite the proximal direction 1, see FIGS. 74. During dose delivery in the assembled state, the clutch mechanism 507 is also in an opened state. Thereby, the first clutch part 508 and the second clutch part 509 are also brought out of engagement by locating them at an axial distance from each other, whereby the second clutch part 509 is shifted in the proximal direction 1 from the first clutch part 508, see FIG. 72.

Furthermore, the adjusting element 518 forms a first clutch member of the clutch mechanism 513 and the sleeve 528 forms a second clutch member of the clutch mechanism 513. The housing 532 of the dose delivery mechanism 554 forms an additional member of the dose delivery mechanism 554 to which the adjusting element 518 is rotationally coupled during dose setting in the assembled state and from which the adjusting element 518 is rotationally decoupled during dose delivery in the assembled state.

The sleeve 528 forms a retaining member for the adjusting element 518.

The dose delivery mechanism 554 comprises a dose definition mechanism 115 that defines the doses settable by a user. Engagement features 116 of the dose definition mechanism 115 are provided at the adjusting element 518, see FIG. 58. The engagement features 116 are configured as radially extending lugs that are flexible in the radial direction. The engagement features 116 engage with corresponding dose stops 118 that are provided at a distal end of the sleeve 528, see FIGS. 63, 64. The dose stops 118 are configured as longitudinally recesses formed at an inner surface of the sleeve 528. During dose setting, the adjusting element 518 and the coupling element 520 are configured to perform more than one full revolution about the longitudinal axis of the dose delivery mechanism 554.

During adjustment of the piston rod 44 in the preassembled state, the dose definition mechanism 115 of the dose delivery mechanism 554 is not active. This is because the engagement features 116 are axially shifted with respect to the dose stops 118 to bring the engagement features 116 and the dose stops 118 out of mutual engagement, see FIG. 75.

With the dose delivery mechanism 554, the housing 532 forms a third element and the first threaded element formed by the piston rod 44 is rotated with respect to that third element during adjustment of the piston rod in the preassembled state, while the second threaded element formed by the dosing member 523 is rotationally fixed with respect to the third element. During dose delivery in the assembled state, the first threaded element formed by the piston rod 44 is rotationally fixed with respect to the third element formed by the housing 532 and the second threaded element formed by the dosing member 523 is rotated with respect to the third element.

With the third injection pen 500, the piston rod 44 can be configured to advance by a first distance into the proximal direction 1 upon proximal movement of the actuation member 520 by a second distance, wherein the second distance is less than 1.5 times the first distance. With the third injection pen 500, the ratio between the second distance and the first distance is given by the ratio of the pitches of the threaded connection 189 between the piston rod 44 and the dosing member 523 and of the further threaded connection 562 between the sleeve 528 and the dosing member 523.

Additionally or alternatively, the third injection pen 500 can also comprise the drug reconstitution unit 56 of the first injection pen 10. Such a third injection pen 500 then can be configured to receive the double chambered cartridge 48 and to perform reconstitution of a lyophilized drug prior to drug delivery.

Embodiments of the third injection pen 500 can be configured, like the first injection pen 10, to restrain a user from prematurely activating the third injection pen 500. Embodiments of the third injection pen 500 can be configured, like the first injection pen 10, to axially lock the actuation member 520 with respect to the housing 532 prior to setting and delivering a first dose. Like the first injection pen 10, the third injection pen 500 can comprise the knob cover 16 and/or the knob key 30.

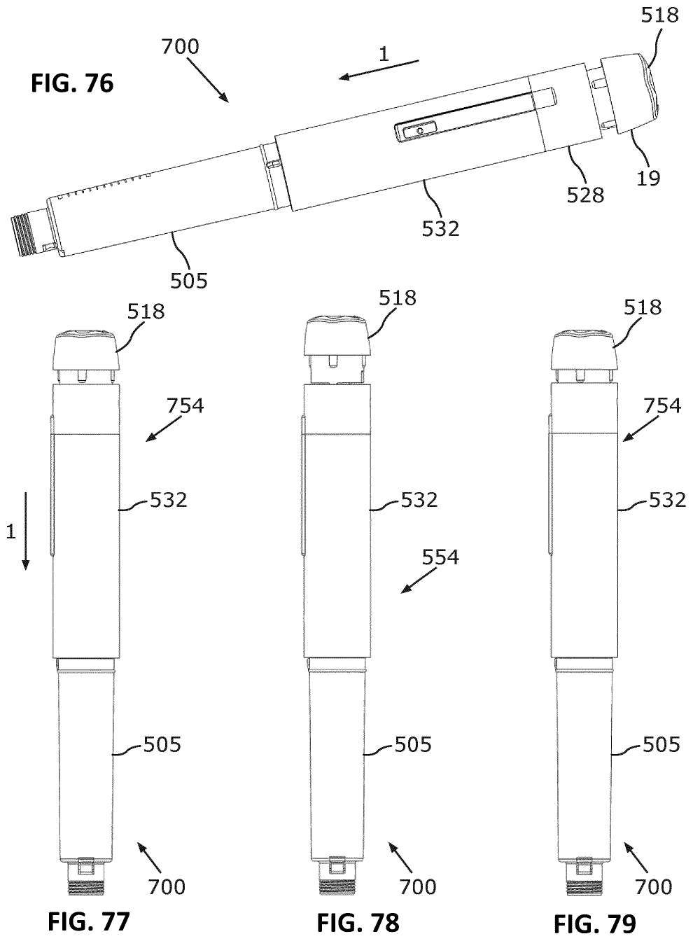

FIG. 76 shows a perspective view of a fourth injection pen 700 according to the present disclosure. The fourth injection pen 700 is a variant of the third injection pen 500. As long as no differences are disclosed in the description or the Figures, the fourth injection pen 700 is configured as it is disclosed for the third injection pen 500. In the following, components of the fourth injection pen 700 that perform the same functions as corresponding components of the third injection pen 500 are labeled with the same reference signs. These components can, however, differentiate among the third injection pen 500 and the fourth injection pen 700 in shape and/or appearance.

FIG. 77 shows a side view of the fourth injection pen 700 in an assembled state during dose setting. Thereby, no dose is set and an adjusting element 518 of a dose delivery mechanism 754 of the fourth injection pen 700 is positioned in an assembled position with respect to a housing 532 of the dose delivery mechanism 754.

FIG. 78 shows a side view of the fourth injection pen 700 in a preassembled state with the adjusting element 518 being in a preassembled position with respect to the housing 532. In the preassembled position, the adjusting element 518 is shifted in a distal direction from its assembled position, whereby the distal direction is orientated perpendicular to a proximal direction 1.

FIG. 79 shows a side view of the fourth injection pen in the preassembled state with the adjusting element 518 in an adjusting position. In the adjusting position, the adjusting element 518 is shifted in the proximal direction compared to the preassembled position.

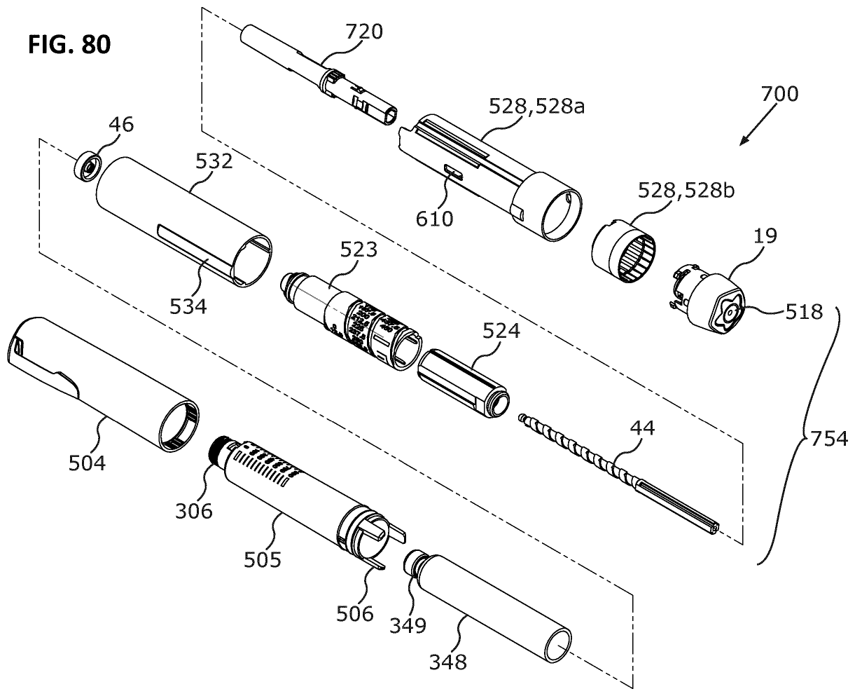

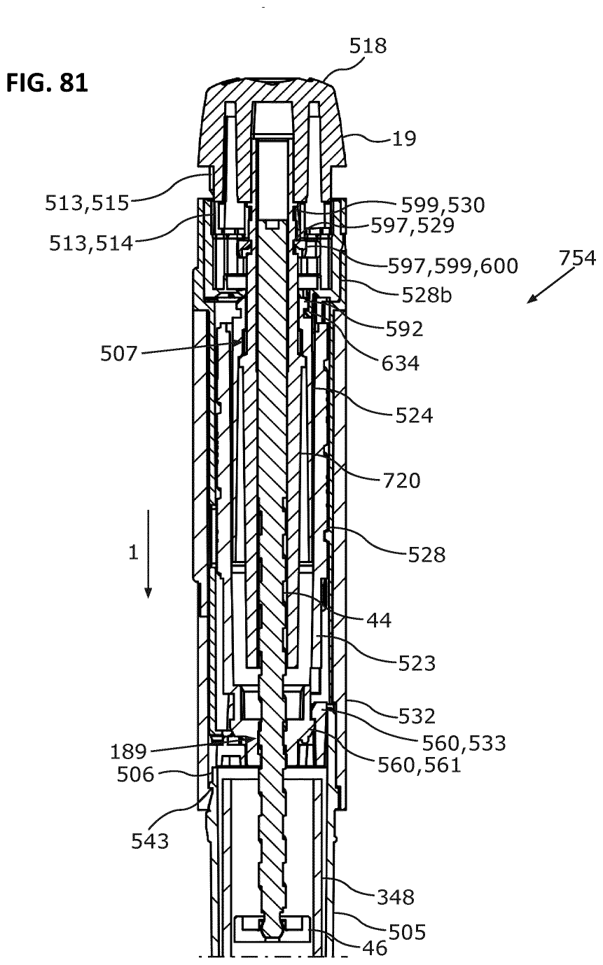

FIG. 80 shows an exploded view of the fourth injection pen 700 and FIG. 81 shows a longitudinal cut through the dose delivery mechanism 754 of the fourth injection pen 700 in the assembled state during dose setting with no dose set.

Like the dose delivery mechanism 554 of the third injection pen 500, the dose delivery mechanism 754 of the fourth injection pen 700 comprises a coupling element 720 that is rotationally fixed and axially movable with respect to a piston rod 44. Unlike the dose delivery mechanism 554, the dose delivery mechanism 754 does not feature the extension 525. Instead, the coupling element 720 directly engages with the piston rod 44 to rotationally fix the coupling element 720 to the piston rod 44 and to allow axial movement between the coupling element 720 and the piston rod 44.

The dose delivery mechanism 754 furthermore comprises a biasing element in the form of a spring, which is not shown in FIGS. 80 and 81. The biasing element biases the adjusting element 518 in the distal direction both in the preassembled state and in the assembled state of the dose delivery mechanism 754.

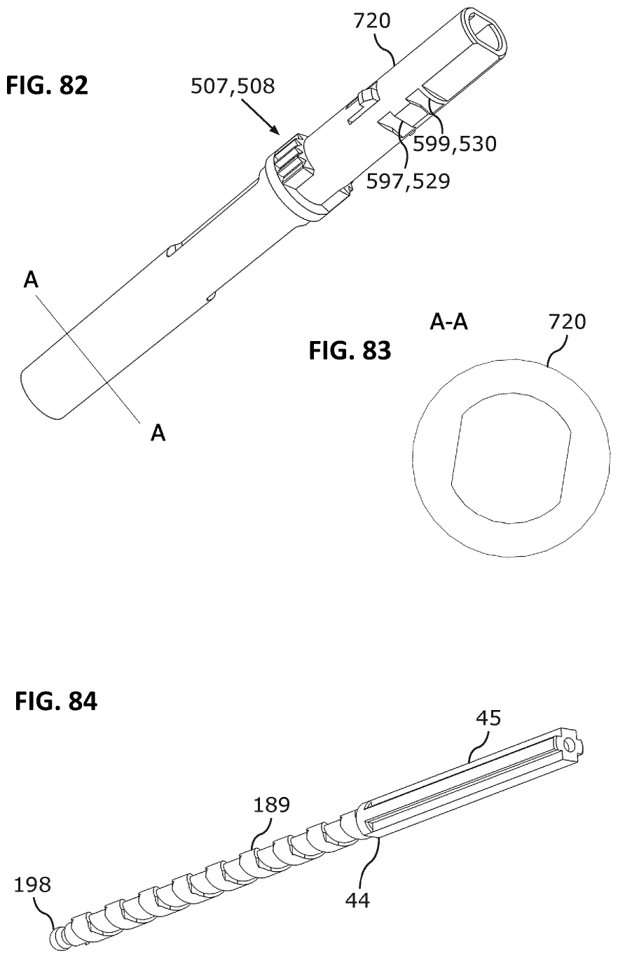

FIG. 82 shows a perspective view of the coupling element 720 of the dose delivery mechanism 754 and FIG. 83 shows a radial cut through the coupling element 720 along the line A-A shown in FIG. 82. The coupling element 720 is configured as a tubular member that extends along the longitudinal direction. It has a non-circular inner cross-section that is configured to receive the piston rod 44. The piston rod 44, which is shown in FIG. 84, has a distal section 45 having an outer shape that is configured to engage with the non-circular inner cross-section of the coupling element 720 to rotationally lock the piston rod 44 and the coupling element 720 and to allow axial movement between the coupling element 720 and the piston rod 44.

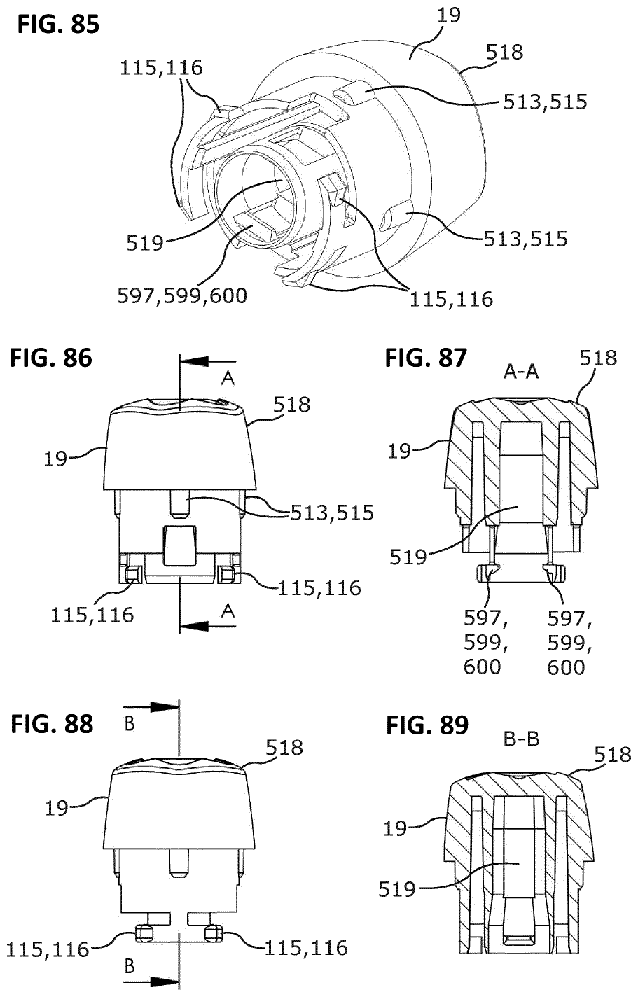

FIGS. 85 to 89 depict the adjusting element 518 of the dose delivery mechanism 754. The adjusting element 518 engages with a distal part of the coupling element 720. The adjusting element 518 thereby is rotationally fixed with respect to the coupling element 720 both in the preassembled state and in the assembled state of the dose delivery mechanism 704. A rotational lock between the coupling element 720 and the adjusting element 518 comprises a non-circular outer cross section of the coupling element 720 that matches and engages with a corresponding inner shape of a central opening 519 of the adjusting element 518.

A latching mechanism 597 acts between the coupling element 720 and the adjusting element 518. In the assembled state of the dose delivery mechanism 754, the latching mechanism 597 prevents the adjusting element 518 from moving distally from the assembled position into the preassembled position with respect to the housing 532. The latching mechanism 597 comprises latch parts 600 formed at the adjusting element 518 and latch counterparts 529 formed at the coupling element 720. The latch parts 600 are configured as flexible hooks that protrude radially inward from the inner surface of the adjusting element 518 at the opening 519. The latch counterparts 529 are configured as recesses located at the outer surface in the distal part of the coupling element 720.

The coupling element 720 can form a retaining member of the dose delivery mechanism 754.

In the preassembled state of the dose delivery mechanism 754, the adjusting element 518 is located at a more distal position with respect to the coupling element 720 than in the assembled state. In this position, the adjusting element 518 is prevented from being detached from the dose delivery mechanism 754 and the coupling element 720 by a further latching mechanism 599. A further latch part of the further latching mechanism 599 is formed by the latch part 600 and a further latch counterpart 530 of the further latching mechanism 599 is formed by an additional recess at the outer surface of the coupling element 720. The further latch counterpart 530 is thereby located at a distal side from the latch counterpart 529.

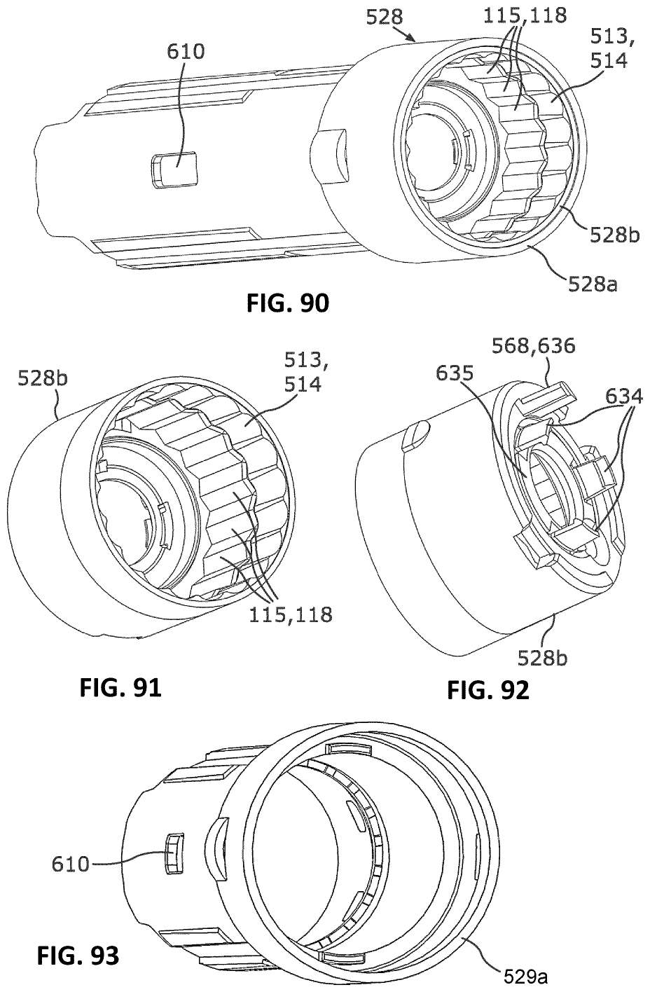

FIGS. 90 to 93 show a sleeve 528 of the dose delivery mechanism 754 that is rotationally fixed and axially movable with respect to the housing 532. The sleeve 528 comprises longitudinal recesses on its outer surface that engage with corresponding longitudinal ridges on an inside surface of the housing 532 to rotationally fix the sleeve 528 to the housing 532. The sleeve 528 comprises an outer part 528a and insert 528b that is rotationally and axially fixed within the outer part 528a at a distal end of the outer part 528a.

A dose definition mechanism 115 of the dose delivery mechanism 754 acts between the adjusting element 518 and the sleeve 528. The dose definition mechanism 115 comprises engagement features 116 that are configured as flexible hooks and provided at a proximal end of the adjusting element 518. The engagement features 116 interact with dose stops 118 provided in a proximal part of an inside surface of the insert 528b of the sleeve 528.

Furthermore, the dose delivery mechanism 754 comprises a clutch mechanism 513 that acts between the adjusting element 518 and the sleeve 528. The clutch mechanism 513 comprises teeth 515 that are located at a proximal outer surface of the adjusting element 518. When closing the clutch mechanism 513, the teeth 515 engage with corresponding teeth 514 provided in a distal part of the inside surface of the insert 528b. The inside surface thereby is a side surface of a cavity formed at the distal end of the insert 528b and the sleeve 528.

The adjusting element 518 forms a first clutch member of the clutch mechanism 513 and the sleeve 528 forms a second clutch member of the clutch mechanism 513. The housing 532 of the dose delivery mechanism 754 forms an additional member of the dose delivery mechanism 754 to which the adjusting element 518 is rotationally coupled during dose setting in the assembled state and from which the adjusting element 518 is rotationally decoupled during dose delivery in the assembled state. Furthermore, the clutch mechanism 513 forms a locking mechanism that rotationally locks the adjusting element 718 to the housing 532 during dose delivery in the assembled state of the dose delivery mechanism 754.

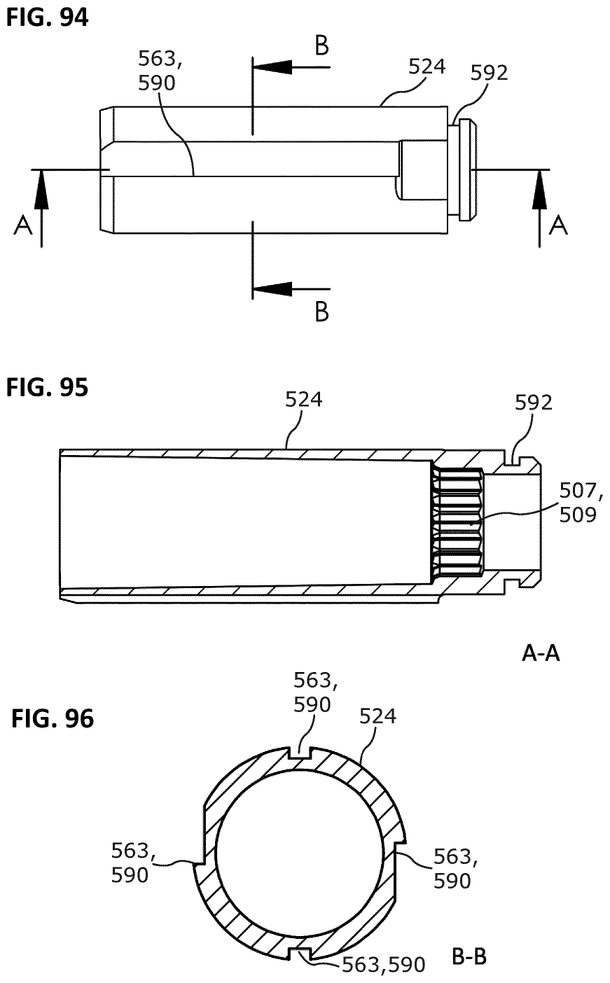

FIGS. 94 to 96 show a coupling member 524 of the dose delivery mechanism 754. The coupling member 524 is axially fixed to the sleeve 528 by a rim 592 provided at a distal end of the coupling member 524. The rim 592 is held between proximal blocking elements 634 and a distal blocking element 635 provided at a proximal end of the insert 528b. The proximal blocking elements 634 are configured as flexible hooks and the distal blocking element 635 is formed by a radial surface of the insert 528b.

A clutch mechanism 507 acts between the coupling member 524 and the coupling element 720, which is received within the coupling member 524. The clutch mechanism 507 comprises a first clutch part 508 that is located on an outside surface of the coupling element 720 and that comprises longitudinal teeth. The clutch mechanism 507 further comprises a second clutch part 509, which is located on an inside surface of the coupling member 524. The second clutch part 509 is configured as longitudinal teeth that mesh with the longitudinal teeth of the first clutch part 508 in the closed state of the clutch.

The coupling element 520 forms a first clutch member of the clutch mechanism 507 and the coupling member 524 forms a second clutch member of the clutch mechanism 507. The dosing member 523 of the dose delivery mechanism 754 forms a further member of the dose delivery mechanism 754 to which the adjusting element 518 is rotationally coupled during dose setting in the assembled state and from which the adjusting element 518 is rotationally decoupled during dose delivery in the assembled state.

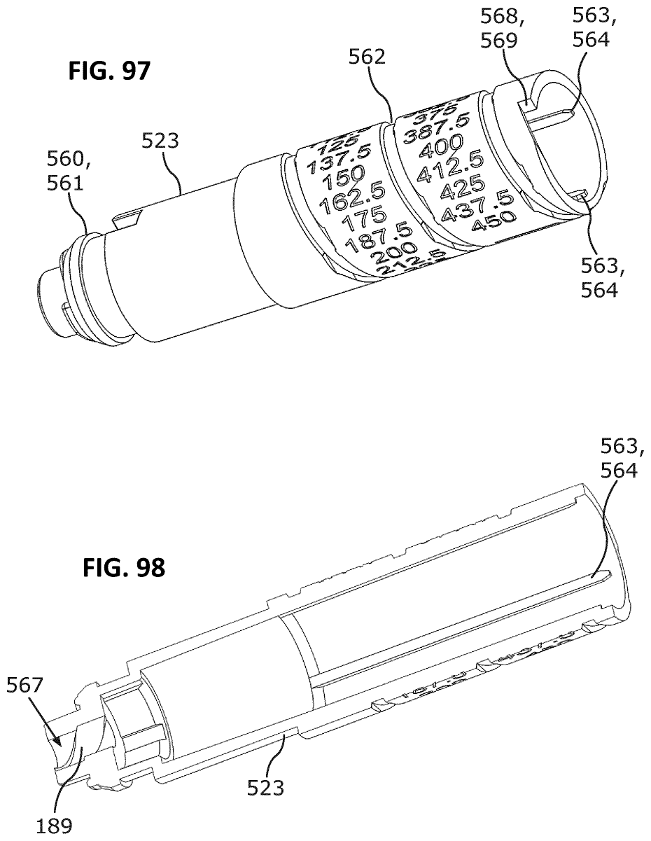

FIGS. 97 and 98 show a dosing member 523 of the dose delivery mechanism 754. A radial stop 568 is provided between the dosing member 523 and the insert 528b of the sleeve 528. The radial stop 568 comprises a stop surface 636 at a proximal extension of the insert 528b and a corresponding stop surface 569 at the distal end of the dosing member 523.

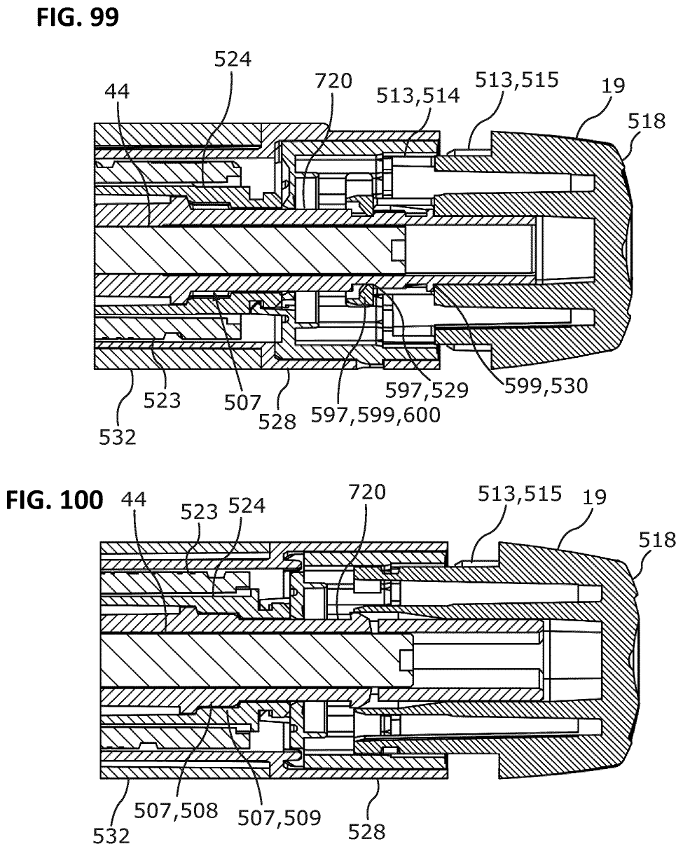

As can be seen from FIGS. 99 and 100, which show the dose delivery mechanism 754 in the assembled state during dose setting with no dose set, the clutch mechanism 507 between the coupling element 720 and the coupling member 524 is closed and the clutch mechanism 513 between the adjusting element 518 and the sleeve 528 is opened during dose setting. Dose setting is then effected by rotating the adjusting element 720 in the same way as it is described for the third injection pen 500.

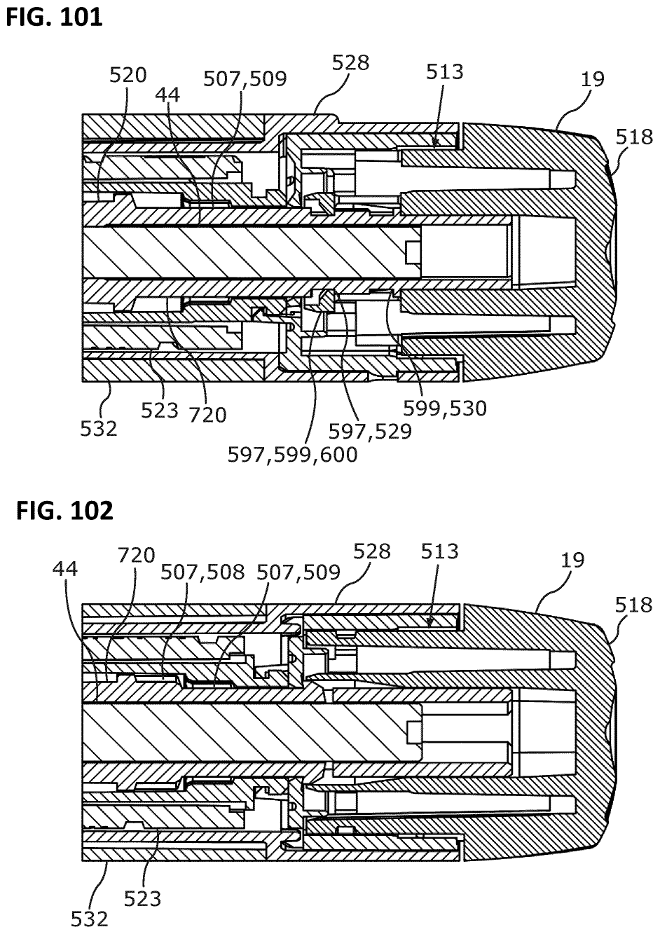

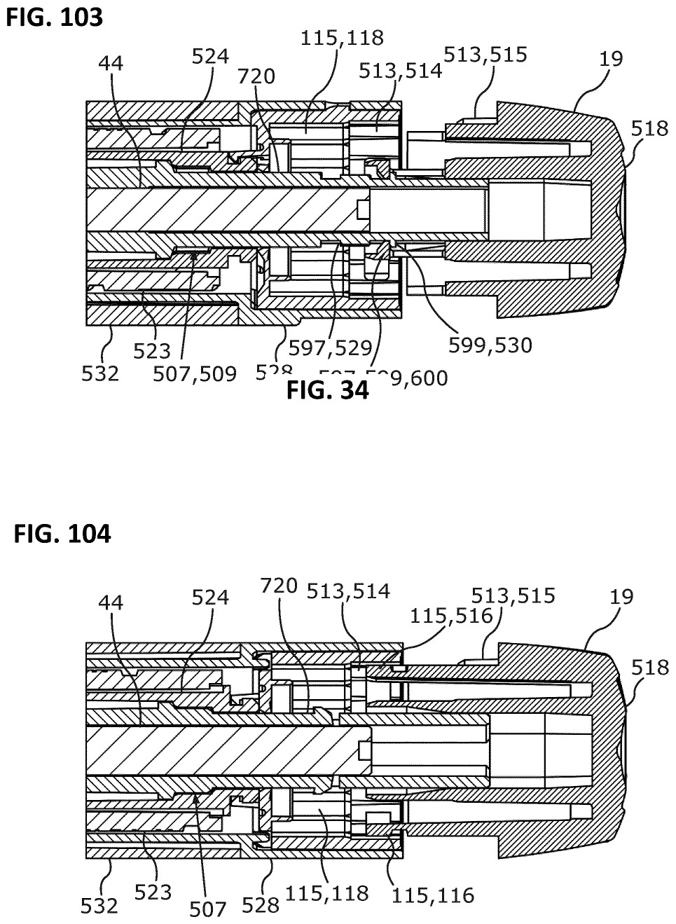

FIGS. 101 and 102 depict the dose delivery mechanism 754 in the assembled state during dose delivery when a set dose has been completely expelled and the user still pushes the adjusting element 518 in the proximal direction 1. During dose delivery, the clutch mechanism 507 is opened, thus allowing rotation between the piston rod 44 and the dosing member 523, and the clutch mechanism 513 is closed, thus rotationally locking the piston rod 44 to the housing 532.

FIGS. 103 and 104 depict the dose delivery mechanism 754 in the preassembled state. Compared to the assembled state, the adjusting element 518 is shifted in the distal direction with respect to the coupling element 720 and the housing 532. The latch part 600 of the adjusting element 518 then engages with the further latch counterpart 530 provided distally from the latch counterpart 529 at the coupling element 720. In the state depicted in FIGS. 103 and 104, the adjusting element 518 is positioned in a preassembled position with respect to the housing 532.

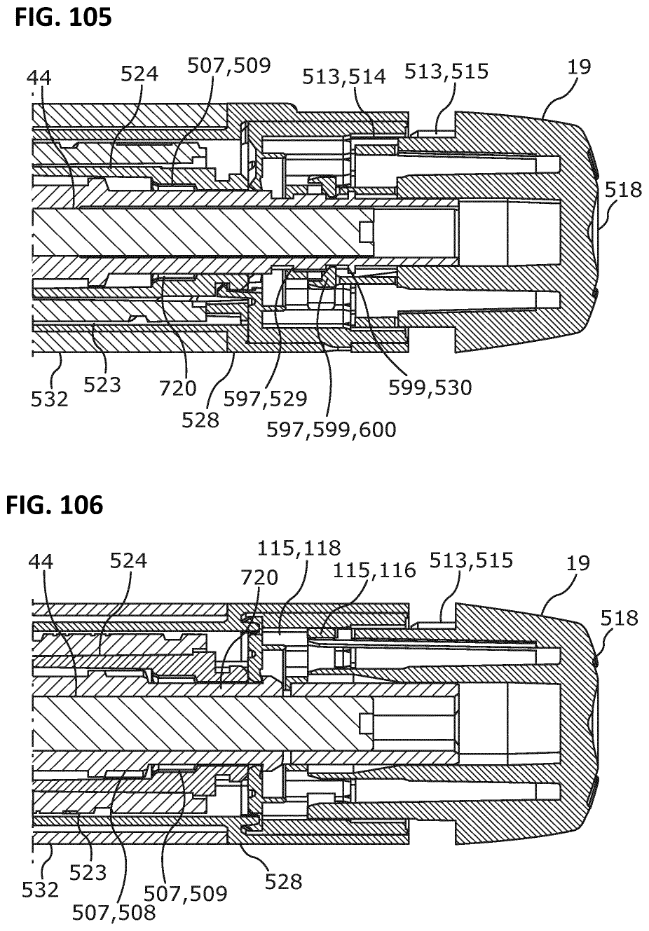

To adjust the position of the piston rod 44 in the preassembled state, the adjusting element 518 is pushed in the proximal direction 1 from the preassembled position into an adjusting position against the biasing force of the spring acting between the adjusting element 518 and the sleeve 528, as it is depicted in FIGS. 105 and 106. Proximal movement of the adjusting element 518 results in proximal movement of the coupling element 720 and in opening of the clutch mechanism 507. At the same time, the clutch mechanism 513 remains opened and the teeth 515 of the adjusting element 518 are prevented from engaging with the teeth 514 of the sleeve 528. This is due to an axial offset between the teeth 513 and the teeth 514.

When an assembler of the fourth injection pen 700 rotates the adjusting element 518 in the adjusting position depicted in FIGS. 105 and 106, the piston rod 44 rotates together with the adjusting element 518 and the dosing member 523 does not rotate due to the opened clutch mechanism 507. Rotation of the piston rod 44 with respect to the dosing member 523 then axially moves the piston rod 44 with respect to the housing 532 via the threaded connection 189.

With the fourth injection pen 700, the piston rod 44 can be configured to advance by a first distance into the proximal direction 1 upon proximal movement of the actuation member 518 by a second distance, wherein the second distance is less than 1.5 times the first distance. With the fourth injection pen 700, the ratio between the second distance and the first distance is given by the ratio of the pitches of the threaded connection 189 between the piston rod 44 and the dosing member 523 and of the further threaded connection 562 between the sleeve 528 and the dosing member 523.

Additionally or alternatively, the fourth injection pen 700 can also comprise the drug reconstitution unit 56 of the first injection pen 10. Such a fourth injection pen 700 then can be configured to receive the double chambered cartridge 48 and to perform reconstitution of a lyophilized drug prior to drug delivery.

Embodiments of the fourth injection pen 700 can be configured, like the first injection pen 10, to restrain a user from prematurely activating the fourth injection pen 700. Embodiments of the fourth injection pen 700 can be configured, like the first injection pen 10, to axially lock the actuation member 520 with respect to the housing 532 prior to setting and delivering a first dose. Like the first injection pen 10, the fourth injection pen 700 can comprise the knob cover 16 and/or the knob key 30.

The mechanism can comprise a dose definition mechanism that allows a user of the device to set at least one dose of medicament for delivery. For example, the dose definition mechanism can be configured to allow only a single predetermined dose to be set. Alternatively, the dose definition mechanism can also be configured to allow a multitude of differing predetermined doses to be set by the user, such as two or more differing doses.

With the injection pens 10, 300, 500, 700, the dose delivery mechanisms 54, 354, 554, 754 each comprises a dose definition mechanism 115, wherein the respective dose definition mechanism 115 acts between the respective dose setting element 18, 318, 518, and the housing 32, 332, 532 during dose setting. The dose definition mechanism 115 thereby has at least one dose stop 118 and a counter element 116, wherein the counter element 116 is configured to rotate with respect to the dose stop 118 when the dose setting element 38, 318, 518 rotates during dose setting and wherein the counter element 116 is configured to engage the dose stop 118 when the dose has been set. The counter elements 116 are formed by the respective engagement features 116 of the dose delivery mechanisms 54, 354, 554, 754.

The dose definition mechanism 115 can define the rotational positions of the dose setting element 22, 518 with respect to the housing 32, 332, 532 that correspond to settable doses. For each settable dose, the dose definition mechanism 115 can comprise a separate dose stop 118. The dose setting element 22, 518 then can be configured to only perform less than a full rotation during dose setting. With the third and fourth injection pen 500, 700, the dose definition mechanisms 115 can also comprise individual dose stops 118 that define more than a single dose, for example, the individual dose stops 118 can be configured to engage with the counter element 116 once upon each full rotation of the counter element 116 with respect to the dose stops 118.

The counter element 116 can be configured as a flexible element that snaps over the dose stop 118 upon setting the dose. For example, the counter element 116 can be configured as a flexible protrusion at a component of the respective dose delivery mechanisms 54, 354, 554, 754. The counter element 116 can, for example, be integrally formed with the component of the respective dose delivery mechanisms 54, 354, 554, 754 it is fixed to. With the first and second injection pen 10, 300, the component is formed by the dosing member 23, 323, with the third and fourth injection pen 500, 700, it is formed by the dose setting element 518.

With the first and second injection pen 10, 300, engagement of the counter element 116 with the dose stop 118 prevents the spring 40 from releasing the energy stored upon rotation of the dose setting element 22 during dose setting. The dose definition mechanism 115 thereby can provide a latching function that keeps the spring 40 in a tensioned state until the dose is delivered by transferring the respective dose delivery mechanism 54, 354 from the dose setting state to the dose delivery state.

With the first and second injection pen 10, 300, the counter element 116 is configured to disengage from the dose stop 118 upon transfer of the respective dose delivery mechanism 54, 354 from the dose setting state into the dose delivery state. This prevents the dose definition mechanism 115 from interfering with the delivery of the set dose. Furthermore, it can allow the spring 40 to release the energy stored upon rotation during dose setting. With embodiments having more than a single dose stop 118, the counter element 116 can be configured to disengage from all dose stops 118 upon transfer of the respective dose delivery mechanisms 54, 354 into the dose delivery state. This can allow the counter element 116 to rotate back to its initial position during dose delivery without interfering with the dose stops 118. The initial position can correspond to a zero dose position in which no dose has been set.

For example, the counter element 116 can be configured to disengage from the dose stop 118 by axially moving with respect to the dose stop 118.

With the injection pens 10, 300, 500, 700, one of the dose stop 118 and the counter element 116, such as the dose stop 118, is rotationally fixed with respect to the housing 32, 332, 532. The one of the dose stop 118 and the counter element 116 then can be axially movable with respect to the dose setting element 22, 518.

With the first and second injection pen 10, 300, the one of the dose stop 118 and the counter element 116, such as the dose stop 118, is axially fixed with respect to the button 18, 318. This allows to move the one of the dose stop 118 and the counter element 116 together with the button 18, 318 upon transfer of the respective dose delivery mechanism 54, 354 from the dose setting state into the dose delivery state. The one of the dose stop 118 and the counter element 116 than can disengage from the other one of the dose stop 118 and the counter element 116 by this movement. For example, the one of the dose stop 118 and the counter element 116 can be linearly guided at the housing 32, 332.

With some embodiments, the one of the dose stop 118 and the counter element 116 is fixed to an outer housing part of the respective dose delivery mechanism 54, 354, 554, 754. The outer housing part can be fixed to a connection for coupling a medicament container 48, 348 to the respective dose delivery mechanism 54, 354, 554, 754. Alternatively, the outer housing part can also be movable with respect to the connection, such as axially movable. Additionally, the outer housing part can be rotationally fixed with respect to the connection. For example, the outer housing part can be a housing connector, such as a housing connector that engages with the housing 32, 332, 532 via an axially movable and rotationally fixed connection. With the first and second injection pen 10, 300, the outer housing part is formed by the dose selector 28, with the third and fourth injection pen 500, 700, it is formed by the sleeve 528.

With the first and second injection pen 10, 300, the dose definition mechanism 115 acts between the dosing member 23, 332 and the housing 32, 332, 532. It then defines rotational positions of the dosing member 23, 332 with respect to the housing 32, 332 that correspond to settable doses.

With the first and second injection pen 10, 300, the other one of the dose stop 118 and the counter element 116, such as the counter element 116, is rotationally fixed with respect to the dosing member 23, 332. Exemplarily, the other one of the dose stop 118 and the counter element 116 is permanently rotationally fixed with respect to the dosing member 23, 332. The other one of the dose stop 118 and the counter element 116 is exemplarily additionally fixed to the dosing member 23, 332, such as to the first part of the dosing member 23, 332 that is movable with respect to the housing 32, 332, 532.

With the first, second, third and fourth injection pen 10, 300, 500, 700, the other one of the dose stop 118 and the counter element 116, such as the counter element 116, is axially movable with respect to the button 18, 318, 518. The one of the dose stop 118 and the counter element 116, such as the dose stop 118, is axially fixed with respect to the button 18, 318, 518. This allows to disengage the dose stop 118 from the counter element 116 by moving the button with respect to the housing 32, 332, 532, as it is the case for the first and second injection pen 10, 300.

With the second injection pen 300, the dose delivery mechanisms 354 comprises a blocking mechanism having a first element and a second element, wherein the first element engages the second element upon release of the button 318 during dose delivery to prevent a transfer of the respective dose delivery mechanism 354 from the dose delivery state to the dose setting state. This keeps the respective dose delivery mechanism 354 in the dose delivery state and prevents a change of the dose setting upon an interruption of dose delivery due to the torque provided by the spring 40. The blocking mechanism can block distal movement of the button 318 against a biasing force biasing the button 318 in the distal direction. The biasing force can be provided by the biasing element 250.

The blocking mechanism is configured to disengage the first element from the second element at a zero dose position at which a set dose has been fully delivered. This allows the dose delivery mechanisms 354 to return to the dose setting state so that a subsequent dose can be set after having completed a previous medicament delivery.

Exemplarily, the first element rotates with respect to the second element in a first direction during dose setting and rotates in a second direction opposite the first direction during dose delivery. Relative movement between the first and second element can bring the first and second element in relative positions that prevent mutual engagement at the end of dose delivery and/or when a dose has been set.

With the second injection pen 300, the first element is configured as the circumferential rib 156 that longitudinally extends around an axis of the housing 332 and the second element is configured as a stop or counter element formed by the engagement feature 116 of the snap element 24 that travels along the circumferential rib 124 during dose delivery.

Exemplarily, the second element passes the first element upon release of the button 318 at the end of dose delivery. For example, the second element can pass through an opening within the first element. The second element can rotate into alignment with the opening at the end of dose delivery.

Additionally or alternatively, the blocking mechanism can be configured to prevent transfer of the dose delivery mechanism 354 from the dose setting state into the dose delivery state unless a dose has been set. Such a blocking mechanism is exemplarily also implemented in the dose delivery mechanism 54 of the first injection pen 10. Exemplarily, the second element passes the first element upon transfer of the dose delivery mechanism 54, 354 from the dose setting state into the dose delivery state. Exemplarily, the second element passes through one of the openings or cut-outs 158 within the first element. The second element exemplarily rotates into alignment with the opening 158 when a dose has been set.

Exemplarily, the second element passes through one of the openings or cut-outs 158 within the first element upon transfer of the mechanism from the dose setting state into the dose delivery state. Such openings 158 prevent blocking and thus allow axial movement of the button 18, 318 to initiate dose delivery.

Exemplarily, the first element of the blocking mechanism and one of the dose stop 118 and the counter element 116, such as the dose stop 118, are fixed to the same member of the dose delivery mechanism 54, 354. Furthermore, the second element of the blocking mechanism and the other one of the dose stop 118 and the counter element 116, such as the counter element 116, are fixed to the same further member of the dose delivery mechanism 54, 354. This allows to precisely align the elements of the blocking mechanism and the elements of the dose definition mechanism 115 and enhances reliability of the dose delivery mechanism 54, 354.

The member of the mechanism that comprises the first element of the blocking mechanism and the one of the dose stop 118 and the counter element 116 exemplarily is the dose selector 28 of the dose delivery mechanism 54, 354. The further member of the dose delivery mechanism 54, 354 that comprises the second element of the blocking mechanism and the other one of the dose stop 118 and the counter element 116 is exemplarily a carrier that is rotationally movable with respect to the dose selector 28. The carrier exemplarily is a part of the dosing member 23, 332, namely by the snap element 24 of the dosing member 23, 323.

The dose selector 28 exemplarily is at least partly located within an outer housing of the dose delivery mechanism 54, 354, namely the housing 32, 332. The dose selector 28 exemplarily is configured to protrude from the outer housing.

Exemplarily, one of the first element and second element of the blocking mechanism and one of the dose stop 118 and the counter element 118 are formed by a single element. As an example, the second element of the blocking mechanism and the counter element 116 of the dose definition mechanism 115 are formed by the single element. This facilitates alignment of the components of the blocking mechanism with respect to the components of the dose definition mechanism 115.

The single element exemplarily is a flexible element that is configured to snap over the dose stop 118 upon rotation with respect to the dose stop 118.

With the first and second injection pen 10, 300, the dose delivery mechanisms 54, 354 comprise a maximum dose mechanism that restrains further rotation of the dose setting element 22 upon dialing past a maximum dose setting, wherein the maximum dose mechanism comprises a maximum dose stop 126 that is exemplarily formed by the hard stop 128 and a blocking part 124 that is exemplarily formed by the hard stop 124 and wherein the blocking part 124 is configured to engage the maximum dose stop 126 128 upon dialing past the maximum dose setting. This provides a well-defined rotational end position of the dose setting element 22. The maximum dose stop 126 128 can also absorb a torque provided by a user and direct the torque to the housing 32, 332 of the dose delivery mechanism 54, 354.

The blocking part 124 exemplarily engages the maximum dose stop 126 128 right at the maximum dose setting. With other embodiments, the blocking part 124 can only engage the maximum dose stop 126 128 after having dialed past the maximum dose setting by a predefined amount.

The blocking part 124 exemplarily is configured as a hard stop that is rigidly connected, such as integrally formed, with a component of the mechanism. Likewise, the maximum dose stop 126 126 exemplarily is configured as such a hard stop.

Exemplarily, the maximum dose stop 126 126 and the blocking part 124 are configured to rotate with respect to each other during dose setting. As an example, one of the maximum dose stop 126 126 and the blocking part 124, namely the maximum dose stop 126 126, can be rotationally fixed with respect to the housing 32, 332 during dose setting and the other one of the maximum dose stop 126 126 and the blocking part 124, such as the blocking part 124, can be rotationally fixed with respect to the dose setting element 22 during dose setting. The other one of the maximum dose stop 126 126 and the blocking part 124 can be rotationally movable with respect to the dose setting element 22 during dose delivery.

Exemplarily, the maximum dose stop 126 is configured as a radial stop and the blocking part 124 is configured to rotate against the maximum dose stop 126 upon dialing past the maximum dose. Such a radial stop provides a well-defined rotational position in which the blocking part 124 and the maximum dose stop 126 get into engagement.

The maximum dose stop 126 and the blocking part 124 exemplarily comprise engagement surfaces that engage with each other. The engagement surfaces exemplarily are orientated essentially perpendicular, namely perpendicular, to a circumferential direction around the longitudinal axis of the dose delivery mechanism 54, 354.

Exemplarily, one of the maximum dose stop 126 and the blocking part 124, such as the maximum dose stop 126, is rotationally fixed with respect to the housing 32, 332. The one of the maximum dose stop 126 and the blocking part 124 exemplarily are permanently rotationally fixed with respect to the housing 32, 332, both during dose setting and dose delivery.

Exemplarily, the one of the maximum dose stop 126 and the blocking part 124 is fixed to an outer housing part of the dose delivery mechanism 54, 354. The outer housing part is exemplarily configured as a housing connector that is located between the dose setting element 22 and the housing 32, 332 of the injection pen 10, 300. The outer housing part is configured as the dose selector 28.

Exemplarily, the other one of the maximum dose stop 126 and the blocking part 124, such as the blocking part 124, is rotationally fixed with respect to the dosing member 23, 332. The other one of the maximum dose stop 126 and the blocking part 124 then rotates with respect to the housing 332, 32 during both dose setting and dose delivery. This allows to reset the maximum dose mechanism during dose delivery. With embodiments, in which a rotational position of the dosing member 23, 332 defines the dose that has been set, rotationally fixing the one of the maximum dose stop 126 and the blocking part 124 to the dosing member 23, 332 precisely defines a maximum dose position in which the maximum dose stop 126 and the blocking part 124 engage with each other.

Exemplarily, the other one of the maximum dose stop 126 and the blocking part 124 is fixed to a coupling member that rotationally couples the dosing member 23, 332 to the dose setting element during dose setting. The coupling member is formed by the snap element 24.

Exemplarily, one of the dose stop 118 and the counter element 116, such as the dose stop 118, and one of the maximum dose stop 126 and the blocking part 124, such as the maximum dose stop 126, are fixed to the same member of the dose delivery mechanism 54, 354. This allows to precisely define the relative positions of the components of the dose definition mechanism 115 with respect to the components of the maximum dose mechanism. The member of the dose delivery mechanism 54, 354 exemplarily is the dose selector 28.

Exemplarily, the other one of the dose stop 118 and the counter element 116, such as the counter element 116, and the other one of the maximum dose stop 126 and the blocking part 124, such as the blocking part 124, are fixed to the same further member of the dose delivery mechanism 54, 354. This also allows to precisely define the relative positions of the components of the dose definition mechanism 115 with respect to the components of the maximum dose mechanism. The further member can, for example, be the carrier that is rotationally movable with respect to the dose selector 28 and that is formed by the snap element 24.

Exemplarily, the dose delivery mechanisms 54, 354 of the first and second injection pen 10, 300 comprise a zero dose mechanism that prevents further axial movement of the nut 38 at the end of dose delivery, wherein the zero dose mechanism comprises a zero dose stop and a further blocking part and wherein the further blocking part is configured to engage the zero dose stop at the end of dose delivery. This provides a well-defined end position for the piston rod 44 at the end of dose delivery and thus contributes to precisely define the amount of medicament delivered.

With the first injection pen 10, the zero dose stop is formed by the end stop 176 at the piston rod 44 guide 42 and the further blocking part is formed by the end stop 174 at the driver 36. With the second injection pen 300, the zero dose stop is formed as a protrusion on the inside surface of the dose selector 28 and the further blocking part is formed by the hard stop 124 at the snap element 24.

The further blocking part 124, 174 is exemplarily configured as a hard stop that is rigidly connected, such as integrally formed, with a component of the dose delivery mechanisms 54, 354. Likewise, the zero dose stop 176 exemplarily is configured as such a hard stop.

Exemplarily, the zero dose stop 176 and the further blocking part 124, 174 are configured to rotate with respect to each other during dose delivery. For example, one of the zero dose stop 176 and the further blocking part 124, 174, such as the zero dose stop 176, is rotationally fixed with respect to the housing 32, 332 during dose setting and the other one of the zero dose stop 176 and the further blocking part 124, 174, such as the further blocking part 124, 174, is rotationally fixed with respect to the dose setting element 22 during dose setting. The other one of the zero dose stop 176 and the further blocking part 124, 174 exemplarily is rotationally movable with respect to the dose setting element 22 during dose delivery.

Exemplarily, the zero dose stop 176 is configured as a radial stop and the further blocking part 124, 174 is configured to rotate against the zero dose stop 176 at the end of dose delivery. Such a radial stop provides a well-defined rotational position in which the further blocking part 124, 174 and the zero dose stop 176 become engaged with each other.

The zero dose stop 176 and the further blocking part 124, 174 exemplarily comprise engagement surfaces that engage with each other. The engagement surfaces can be orientated essentially perpendicular, such as perpendicular, to a circumferential direction around the longitudinal axis of the dose delivery mechanism 54, 354.

Exemplarily, one of the zero dose stop 176 and the further blocking part 124, 174, such as the zero dose stop 176, is rotationally fixed with respect to the housing 32, 332. The one of the zero dose stop 176 and the further blocking part 124, 174 exemplarily is permanently rotationally fixed with respect to the housing 32, 332, both during dose setting and dose delivery.

Exemplarily, the one of the zero dose stop 176 and the further blocking part 124, 174 is fixed to an outer housing part of the dose delivery mechanism 54, 354. With the second injection pen 300, the outer housing part exemplarily is configured as a housing connector that is located between the dose setting element 22 and the housing 332 of the second injection pen 300. Also the first injection pen 10 can alternatively comprise such a zero dose stop 176.

Exemplarily, the other one of the zero dose stop 176 and the further blocking part 124, 174 is rotationally fixed with respect to the dosing member 23, 332. The other one of the zero dose stop 176 and the further blocking part 124 then rotates with respect to the housing 32, 332 during both dose setting and dose delivery. This allows to reset the zero dose mechanism during dose setting. With embodiments, in which a rotational position of the dosing member 23, 332 defines the dose that has been set, rotationally fixing the one of the zero dose stop 176 and the further blocking part 124, 174 to the dosing member 23, 332 precisely defines a zero dose position in which the zero dose stop and the further blocking part 124 engage with each other.

With the second injection pen 200, the other one of the zero dose stop 176 and the further blocking part 124, 174 is fixed to a coupling member that rotationally couples the dosing member 23, 332 to the dose setting member 22 during dose setting. The coupling member is formed by the snap element 24 of the dosing member 323. Also the first injection pen 10 can comprise such a configuration.

Exemplarily, one of the dose stop 118 and the counter element 116 of the second injection pen 300, such as the dose stop 118, and one of the zero dose stop and the further blocking part 124 of the second injection pen 300, such as the zero dose stop, are fixed to the same member of the dose delivery mechanism 354. This allows to precisely define the relative positions of the components of the dose definition mechanism 115 with respect to the components of the zero dose mechanism. The member of the dose delivery mechanism 354 exemplarily is the dose selector 28. Also the first injection pen 10 can have such a configuration.

Exemplarily, the other one of the dose stop 118 and the counter element 116 of the second injection pen 300, such as the counter element 116, and the other one of the zero dose stop and the further blocking part 124 of the second injection pen 300, such as the further blocking part 124, are fixed to the same further member of the dose delivery mechanism 354. This also allows to precisely define the relative positions of the components of the dose definition mechanism 115 with respect to the components of the zero dose mechanism. The further member can, for example, be the carrier that is rotationally movable with respect to the dose selector 28. Also the first injection pen 10 can have such a configuration.

Exemplarily, the second injection pen 300, one of the maximum dose stop 126 and the blocking part 124 of the maximum dose mechanism and one of the zero dose stop and the further blocking part 124 of the zero dose mechanism, such as the maximum dose stop 126 and the zero dose stop, are fixed to the same member of the mechanism. Furthermore, the other one of the maximum dose stop 126 and the blocking part 124 of the maximum dose mechanism and the other one of the zero dose stop and the further blocking part 124 of the zero dose mechanism, such as the blocking part 124 and the further blocking part 124, are fixed to the same further member of the mechanism. This provides for a precise alignment between the components of the maximum dose mechanism and the components of the zero dose mechanism. Also the first injection pen 10 can have such a configuration.

Irrespective of whether they are fixed to the same member of the mechanism or not, the maximum dose stop 126 and/or the minimum dose stop can be integrally formed with the member they are fixed to. Analogously, the blocking part 124 and/or the further blocking part 124 can be integrally formed with the member they are fixed to.

Exemplarily, with the second injection pen 300, the blocking part 124 of the maximum dose mechanism forms the further blocking part 124 of the minimum dose mechanism. This allows to precisely define the distance between the maximum dose position and the zero dose position. Also the first injection pen 10 can have such a configuration.

In general, the dose delivery mechanisms 54, 354, 554, 754 can comprise a clutch mechanism 113 having a first engaging part and a second engaging part, wherein the clutch mechanism 113 is closed during one of dose setting and dose delivery and opened during the other one of dose setting and dose delivery. The clutch mechanism 113 is in the opened state when the first engaging part and the second engaging part do not engage with each other and the clutch mechanism 113 is in the closed state when the first engaging part and the second engaging part engage with each other.

Exemplarily, the dose delivery mechanisms 54, 354 of the first and second injection pen comprise a clutch mechanism 113, wherein the clutch mechanism 113 rotationally locks the nut 38 to the piston rod 44 during dose delivery and rotationally releases the nut 38 from the piston rod 44 during dose setting. By rotationally locking the nut 38 to the piston rod 44, the clutch mechanism 113 forces simultaneous proximal movement of the nut 38 together with the piston rod 44. Exemplarily, the clutch mechanism 113 rotationally locks the threaded connection 189 that couples the nut 38 to the piston rod 44. The clutch mechanism 113 locks the nut 38 to the piston rod 44 in a closed state and rotationally release the nut 38 from the piston rod 44 in an opened state.

The clutch mechanism 113 exemplarily comprises a first engaging part and a second engaging part and the first and second engaging parts are configured to engage with each other to rotationally lock the nut 38 to the piston rod 44. The first engaging part and the second engaging part exemplarily are configured to become disengaged from each other by relative axial movement with respect to each other. The first engaging part is exemplarily formed by the teeth 114 of the connector 26 and the second engaging part is exemplarily formed by the teeth 108 of the dose setting element 22.

The clutch mechanism 113 exemplarily is configured to be transferred from the opened state into the closed state upon movement of the button 18, 318 and transfer of the dose delivery mechanisms 54, 354 from the dose setting state to the dose delivery state. One of the first engaging part and the second engaging part, such as the second engaging part, is axially fixed with respect to the button 18, 318 and the other one of the first engaging part and the second engaging part, such as the first engaging part, is axially fixed with respect to the housing 32, 332. Additionally or alternatively, the first engaging part is axially fixed with respect to the dosing member 23, 332.

Exemplarily, the first engaging part is rotationally fixed to the housing 32, 332 and the second engaging part is rotationally fixed to the nut 38.

The second engaging part exemplarily is rotationally fixed to the button 18, 318 and/or the dose setting element 22.

Exemplarily, the clutch mechanism 113 rotationally fixes the nut 38 to the piston rod 44 during dose delivery via the housing 32, 332 and, for example, via the dose setting element 22 and/or the button 18, 318.

Exemplarily, the clutch mechanism 113 acts between the button 1, 318 and the housing 32, 332 and/or between the dose setting element 22 and the housing 32, 332.

Exemplarily the button 18, 318 is rotationally coupled, such as permanently rotationally coupled, to one of the first engaging part and the second engaging part. As an example, the button 18, 318 constitutes the one of the first engaging part and the second engaging part.

Exemplarily, the dose delivery mechanisms 54, 354 comprise a further clutch mechanism 107, wherein the further clutch mechanism 107 rotationally locks the dose setting element 22 to one end of the spring 40 during dose setting and decouples the dose setting element 22 from the one end of the spring 40 during dose delivery. Furthermore, the further clutch mechanism 107 has a further first engaging part and a further second engaging part, wherein the further first engaging part is configured to move into engagement with the further second engaging part to rotationally lock the dose setting element 22 to the one end of the spring 40. The further clutch mechanism 107 allows to tension the spring 40 during dose setting and at the same time prevents the dose setting element 22 from rotating during dose delivery when the spring 40 relaxes again. The further first engaging part is exemplarily formed by the teeth 110 at the snap element 23 and the further second engaging part is exemplarily formed by the teeth 108 at the button 22.

The further clutch mechanism 107 exemplarily is transferred from a closed state, in which the further first engaging part engages with the further second engaging part, into an opened state, in which the further first engaging part is disengaged from the further second engaging part, by movement of the button 18, 318. The movement of the button 18, 318 exemplarily is the movement that transfers the dose delivery mechanisms 54, 354 from the dose setting state into the dose delivery state.

Exemplarily, one of the further first engaging part and the further second engaging part, such as the further second engaging part, is rotationally and axially fixed to the dose setting element 22. This allows to open and close the further clutch mechanism 107 by relative movement of the dose setting element 22 with respect to the other one of the further first engaging part and the further second engaging part.

Exemplarily, the one of the further first engaging part and the further second engaging part is rotationally and axially fixed to the button 18, 318. The dose setting element 22 thereby is at least rotationally fixed to the button 18, 318.

Exemplarily, the further clutch mechanism 107 acts between the dosing member 23, 332 and the dose setting element 22. The dosing member 23, 332 then can rotationally couple the dose setting element 22 to the one end of the spring 40.

Exemplarily, one of the further first engaging part and the further second engaging part, such as the further first engaging part, is rotationally fixed to the dosing member 23, 332. Exemplarily, the one of the further first engaging part and the further second engaging part can be axially fixed to the dosing member 23, 332.

Exemplarily, the clutch mechanism 113 comprises the first engaging part that engages the second engaging part to rotationally fix the nut 38 to the piston rod 44 during dose delivery, wherein the further second engaging part of the further clutch mechanism 107 forms the first engaging part of the clutch mechanism 113. This provides a compact construction of the clutch mechanisms 107, 113.

The nut 38 exemplarily is rotationally fixed with respect to the button 18, 318 and/or the dose setting element 22. As an example, the nut 38 is rotationally fixed and axially movable with respect to the button 18, 318 and/or the dose setting element 22. It is coupled to the button 18, 318 and/or the dose setting element 22 by a rotation lock. The rotation lock is formed by the nut 38 and one of the button 18, 318 and the dose setting element 22, such as the button 18, 318.

The nut 38 is rotationally movable with respect to the piston rod 44 when the dose delivery mechanisms 54, 354 are in the dose setting state and the nut 38 is rotationally fixed with respect to the piston rod 44 when the dose delivery mechanisms 54, 354 are in the dose delivery state. Rotation of the nut 38 with respect to the piston rod 44 during dose setting leads to axial movement due to the threaded connection between the nut 38 and the piston rod 44. By rotationally locking the nut 38 to the piston rod 44 during dose delivery, the threaded connection between the nut 38 and the piston rod 44 is blocked and the nut 38 and the piston rod 44 become axially fixed with respect to each other.

The nut 38 is turned by the dose setting element 22 during dose setting and performs an axial movement due to the threaded connection to the piston rod 44. The rotation of the nut 38 causes the nut 38 to translate axially in the distal direction along the thread located on the piston rod 44 during dose setting and to translate in the proximal direction during dose cancellation. Axial movement of the nut 38 with respect to the piston rod 44 defines the axial movement of the piston rod 44 during dose delivery and thus the amount of medicament expelled during dose delivery.

Some general remarks concerning the disclosure:

The second threaded connection 170, 172 can be provided between the driver 36 and the piston rod guide 42. However, there are multiple other options where the second threaded connection 170, 172 could be provided. For example, the second threaded connection could be provided between the dosing element 34 and the housing 32. The second threaded connection 170, 172 preferably acts between any part that is rigidly connected to the driver 36 and any part that is rigidly connected to the housing 32.

Preferably, the actuation member 18, 20, 22 is coupled via the nut 38 to the piston rod 44 in a way that an axial movement of the actuation member 18, 20, 22 during dose delivery causes an axial movement of the nut 38 and the piston rod 44. To apply an axial force from the nut 38 to the piston rod 44 to move the piston rod 44 in the axial direction via the nut 38 during dose delivery, the nut 38 forms a first threaded connection with the pistion rod 44 via the outer thread 190 meshing with the inner thread 192. The nut 38 and the piston rod 44 are blocked from relative rotational movement during dose delivery so that an axial movement of the nut 38 causes an axial movement of the piston rod 44.

Regarding the optional feature that “all parts that are configured to rotate relative to the housing 32 during dose delivery, are connected to the housing 32 via exactly one threaded connection 170, 172, it is pointed out that the dose delivery mechanism 54 can comprise a driver 36, a dose sleeve 34 and/or a snap element 24. One or more of these parts can be configured to rotate relative to the housing 32 during dose delivery. Furthermore, the delivery mechanism 54 can have one or more further parts that are configured to rotate relative to the housing 32 during dose delivery. All parts of the delivery mechanism 54 that are configured to rotate relative to the housing 32 during dose delivery can form a sub-assembly. This sub-assembly can form exactly one threaded connection 170, 172 with the housing 32 or any part rigidly connected to the housing 32.

The coupling means 100, 102 can be formed on the injection button and the snap element 24. However, the coupling means 100 can alternatively be formed on the snap ring 20 or the dose setting knob 22. The coupling means 102 could generally be formed on any part that is not axially movable relative to the housing. The person skilled in the art understands that there are multiple options where to place the coupling means 100, 102. If the coupling means 100, 102 are configured to permanently axially lock the dose setting knob 22 to the dose setting device or the housing 50, this can be done by multiple ways, e.g. by a permanent form fitted connection or by a permanent friction fitted connection.

Generally, the dose setting mechanism can comprise a clutch that connects a nut 38 to a piston rod 44, with the nut 38 being rotationally fixed to the piston rod 44 during dispensing and rotational relative to the piston rod 44 during dose setting. Preferably, rotating the dose setting knob effects rotation of the nut 38 relative to the piston rod 44 during dose setting. There are multiple ways to design a connection between the dose setting knob and the nut 38.

Generally, the piston rod 44 can be rotationally fixed with respect to the housing during dose delivery/dispensing.

The proximal end 14 comprises a dispensing outlet. The distal end 12 is arranged at an opposite end of the proximal end 14. The dispensing outlet can comprise one of a needle, a cannula, and a point of connection for a needle or cannula.

The cover 16 can cover the part of the dose delivery activation mechanism configured to activate dose delivery. The dose delivery activation mechanism are the parts that are configured to permit a dose dispensing from the delivery device. The activation mechanism can comprise the injection button 18 forming a distal end face of the injection pen. The cover 16 can also cover the dose setting knob 22. Thereby, the cover 16 prevents setting the dose and/or activating the injection before a mixing of the two components is done.

The as-delivered condition is a condition in which all parts of the dose delivery mechanism 54 are arranged at the same relative position with respect to one another as they are following manufacture of the dose delivery mechanism 54. In other words, the as-delivered condition is a condition in which the user has not rotated or otherwise changed the position of the dose setting member 34 relative to the housing. Hence, when a dose has been set by the user, the dose delivery mechanism 54 is not in an “as-delivered condition” anymore but instead in a “set state”. According to an embodiment, in the “as-delivered condition”, the dose setting knob 22 is not rotatable towards a zero-dose position. According to an embodiment, the actuation is blocked in the “as-delivered condition” to prevent accidental activation.

The dose delivery mechanism can comprise the activation member 18, the dose setting knob 22, the snap element 24, the dose selector 28 and/or the clip 30. The activation member 18 can be configured to to be pushed by the user to start the injection process. The snap element 24 is preferably configured to engage with the dose selector 28, preferably in different rotational positions, to define a settable dose. The clip 30 can be used in combination with the knob cover 16 to avoid movement of the activation member 18 relative to the housing 32 when the pen drops onto its proximal end 14.

1. A cartridge holding unit comprising:
a cartridge container forming an outer surface of the cartridge holding unit and having an inner space; and
a cartridge holder forming a tube-shaped holding section arranged in the inner space of the cartridge container,
the cartridge holder is configured to hold a cartridge in parallel with the cartridge holder, and
the cartridge holder and the cartridge container are coupled to each other in respective axial orientations.

2. The cartridge holding unit according to claim 1,
wherein the cartridge container and the cartridge holder are formed as two separate parts.

3. The cartridge holding unit according to claim 2,
wherein the cartridge container and the cartridge holder are non-detachably coupled to each other.

4. The cartridge holding unit according claim 1,
wherein the cartridge container and the cartridge holder are axially fixedly connected to each other.

5. The cartridge holding unit according to claim 4,
wherein the cartridge container and the cartridge holder are axially fixedly connected to each other by a form-locked engagement.

6. The cartridge holding unit according to claim 1,
wherein the cartridge container and the cartridge holder are firmly coupled to each other in the respective axial orientations by a form-locked engagement.

7. The cartridge holding unit according to claim 1,
wherein the cartridge holder forms a first axial fixation element configured to engage a second axial fixation element of the cartridge container,
one of the first and second axial fixation elements is a circumferentially extending groove and the other one of the first and second axial fixation elements is a circumferentially extending rib.

8. The cartridge holding unit according to claim 1,
wherein the cartridge holder and the cartridge container are rotationally fixed to each other.

9. The cartridge holding unit according to claim 8,
wherein the cartridge holder forms a first rotation fixation element that engages a second rotation fixation element of the cartridge container.

10. The cartridge holding unit according to embodiment 9,
wherein the cartridge holder and the cartridge container are rotationally fixed to each other by a form-fitting engagement between the first rotation fixation element of the cartridge holder and the second rotation fixation element of the cartridge container,
one of the first and second rotation fixation elements is a radially protruding rib and the other one of the first and second rotation fixation elements is a radially extending groove configured to receive the rib.

11. The cartridge holding unit according to claim 1,
wherein the cartridge holder forms a needle connector, such as a thread, at a proximal end.

12. The cartridge holding unit according to claim 11,
wherein the needle connector surrounds an opening at a proximal end of the cartridge holder, and
the opening is configured to receive a needle that is in fluid connection with an interior of the cartridge inserted into the cartridge holder.

13. The cartridge holding unit according to claim 1,
wherein the cartridge holder forms a slot extending in an axial direction, the slot reversibly widening the cartridge holder to enable axially insertion of a cartridge.

14. The cartridge holding unit according to embodiment 13,
wherein the slot extends until a distal end of the cartridge holder.

15. The cartridge holding unit according to claim 1,
wherein, when the cartridge holder is firmly coupled to the cartridge container, a first window formed in the cartridge holder is aligned with a second window in the cartridge container so that a cartridge arranged inside the cartridge holder is viewable through the first and second windows.

16. The cartridge holding unit according to claim 1,
wherein the holding section of the cartridge holder defines a cylindrical receptacle.

17. The cartridge holding unit according to claim 1,
wherein an inner surface of the cartridge holder is configured to be in areal contact with the cartridge inserted into the cartridge holder.

18. The cartridge holding unit according to claim 1,
wherein the cartridge holder forms a cut-out to receive a protrusion of the cartridge when inserted.

19. The cartridge holding unit according to claim 18,
wherein the cut-out is circumferentially offset from a longitudinal slot of the cartridge holder radially opposite of the longitudinal slot of the cartridge holder.

20. The cartridge holding unit according to claim 18,
wherein the cut-out is a window in the tube-shaped holding section.

21. The cartridge holding unit according to claim 18,
wherein the cut-out includes borders configured to fully enclose a protrusion on an outer surface of the cartridge to prevent rotation of the cartridge around a longitudinal axis or axial movement of the cartridge along the longitudinal axis when inserted.

22. The cartridge holding unit according to claim 1,
wherein the holding section is configured to extend over at least 50% of a longitudinal length of the cartridge when inserted.

23. The cartridge holding unit according to claim 1,
wherein the cartridge holder has a connection element to connect to a dosing mechanism.

24. The cartridge holding unit according to claims 23,
wherein the connection element comprises a thread.

25. The cartridge holding unit according to claim 23,
wherein the connection element extends into an annular space in between the cartridge holder and the cartridge container.

26. A cartridge unit comprising:
the cartridge holding unit according to claim 1; and
the cartridge inserted into the cartridge holder.

27. A reconstitution unit comprising:
the cartridge holder unit according to claim 1; and
a connection section of a dosing mechanism,
the cartridge container forms a first thread and the connection section of the dosing mechanism forms a corresponding second thread engageable with each other to cause an axial movement of the cartridge container relative to the connection section of the dosing mechanism by rotating the cartridge container relative to the connection section of the dosing mechanism.

28. The reconstitution unit according to claim 27,
wherein the first thread is formed on an inner circumferential surface of the cartridge container and the second thread is formed on an outer circumferential surface of the connection section of the dosing mechanism.

29. The reconstitution unit according to claim 27,
further comprising the cartridge having a protrusion,
the cartridge holder forming a cut-out to receive the protrusion of the cartridge, and
the protrusion is configured to form-fittingly engage the cut-out so that the cartridge is axially or rotationally fixed to the cartridge holder.

30. The reconstitution unit according to claim 27,
wherein a space is formed between an outer circumferential surface of the cartridge holder and an inner circumferential surface of the cartridge container so that the connection section of the dosing mechanism is capable of being arranged in the space between the outer circumferential surface of the cartridge holder and the inner circumferential surface of the cartridge container upon mounting the cartridge holding unit to the connection section of the dosing mechanism.
